(19) United States
(12) Patent Application Publication
(10) Pub. No.: US 2024/0002521 A1
(43) Pub. Date: Jan. 4, 2024

(54) ANTI-CD25 ANTIBODIES

(71) Applicants: INSERM (INSTITUT NATIONAL DE LA SANTÉ ET DE LA RECHERCHE MÉDICALE), Paris Cedex 13 (FR); INSTITUT JEAN PAOLI & IRENE CALMETTES, Marseille (FR); ALDERAAN BIOTECHNOLOGY, Paris (FR); UNIVERSITE D'AIX MARSEILLE, Marseille Cedex 7 (FR); UNIVERSITÉ DE PARIS, Paris (FR); UNIVERSITE PARIS EST CRETEIL VAL DE MARNE, Créteil Cedex (FR); AL DE LA RECHERCHE SCIENTIFIQUE, Paris (FR)

(72) Inventors: Daniel OLIVE, Marseille Cedex 09 (FR); Armand BENSUSSAN, Paris Cedex 10 (FR); Jérôme GIUSTINIANI, Créteil (FR); Anne MARIE-CARDINE, Paris Cedex 10 (FR); Amaud FOUSSAT, Paris (FR); Jemila HOUACINE, Pais (FR)

(73) Assignees: INSERM (INSTITUT NATIONAL DE LA SANTÉ ET DE LA RECHERCHE MÉDICALE), Paris Cedex 13 (FR); INSTITUT JEAN PAOLI & IRENE CALMETTES, Marseille (FR); ALDERAAN BIOTECHNOLOGY, Paris (FR); UNIVERSITE D'AIX MARSEILLE, Marseille Cedex 7 (FR); UNIVERSITÉ PARIS CITÉ, Paris (FR); UNIVERSITE PARIS EST CRETEIL VAL DE MARNE, Créteil Cedex (FR)

(21) Appl. No.: 18/253,469

(22) PCT Filed: Nov. 19, 2021
(86) PCT No.: PCT/EP2021/082381

§ 371 Date: May 18, 2023

(30) Foreign Application Priority Data
Nov. 20, 2020 (EP) 20306421.7

(51) Int. Cl. C07K 16/28

(52) U.S. Cl. CPC C07K 16/2866; C07K 2317/21; C07K 2317/732

(57) ABSTRACT

Novel anti-human CD25 antibodies and their use for treating cancer and infectious diseases. Also, a nucleic acid encoding an anti-human CD25 antibody or an antigen-binding fragment thereof, and a fusion protein including an isolated anti-human CD25 antibody or an antigen-binding fragment thereof. Further, a pharmaceutical composition including an isolated anti-human CD25 antibody or an antigen-binding fragment thereof.

### FIELD OF INVENTION

The present invention relates to the field of treatment of cancer and infectious diseases, and in particular discloses novel anti-human CD25 antibodies that may be used for treating cancer and infectious diseases.

### BACKGROUND

Regulatory T cells (Tregs) are key mediators of immune tolerance, involved in the protection of the body against autoimmunity. However, in cancer, Tregs appear to play a controversial role. Indeed, tumor microenvironment may favor differentiation and recruitment of Tregs, which may thus suppress antitumor effector T cell function. Tregs may thus constitute a major obstacle for immunotherapy. This phenomenon has been described in many human cancers and in most mouse models of tumor growth, wherein the frequency of Tregs and their suppressor functions are increased as compared to those reported for healthy subjects. In particular, it has been shown that Tregs accumulate in the tumor in the presence of tumor-derived chemokines, and once in place, proceed to prevent or blunt antitumor responses mediated by immune cells infiltrating the tumor microenvironment. Therefore, accumulation of Tregs may participate to the escape of the tumor from the host immune system by silencing antitumor immune effector cells.

Consequently, there is a need to develop means for silencing or eliminating Tregs from tumors, thereby restoring antitumor functions of T effector cells, and treating cancer.

Tregs were first described by Sakaguchi et al. as a circulating subset of murine CD4⁺ T cells expressing constitutively high levels of CD25, the interleukin-2 receptor α chain that binds to interleukin-2 (IL-2) and regulates development and homeostasis of Tregs.

Anti-CD25 antibodies, and the use thereof for modulating Tregs function or activity, were thus described in the art. For example, Basiliximab is a chimeric mouse-human CD25 antibody, that may be used for preventing graft versus host diseases. In addition, Daclizumab is a chimeric mouse-human CD25 antibody approved for the treatment of relapsing forms of multiple sclerosis.

However, to the Applicants knowledge, no anti-CD25 antibodies has been clinically validated for treating cancer in humans. There remains thus still a need for effective antitumoral immunotherapies, based on the modulation of the IL-2/CD25 pathway.

### SUMMARY

The present invention relates to an isolated anti-human CD25 antibody or antigen-binding fragment thereof, wherein the variable region of the heavy chain (VH) comprises the three following complementary-determining regions (CDRs):

CDR1:
(SEQ ID NO: 1)
X₁HAMA,
wherein X₁ is N or S;
CDR2:
(SEQ ID NO: 2)
YISYDGDNTYYX₂DSVKG
wherein X₂ is R or A;
and
CDR3:
(SEQ ID NO: 3)
GGNSGYD;

or any CDR having an amino acid sequence that shares at least about 70% of identity with SEQ ID NO: 1-3; and wherein the variable region of the light chain (VL) comprises the three following CDRs:

CDR1:
(SEQ ID NO: 4)
X₃X₄SQNVNKFLN
wherein X₃ is K or R
and
wherein X₄ is G or A;
CDR2:
(SEQ ID NO: 5)
GTNSLQX₅
wherein X₅ is T or S;
and
CDR3:
(SEQ ID NO: 6)
QQYTSWPWT,

or any CDR having an amino acid sequence that shares at least about 70% of identity with SEQ ID NO: 4-6; and

wherein when X₁ is N and X₂ is R, X₃ is R and/or X₅ is S; and

wherein when X₃ is K and X₅ is T, X₁ is S and/or X₂ is A.

In one embodiment, the VH of said antibody or antigen-binding fragment thereof comprises the following CDRs:

CDR1:
(SEQ ID NO: 7)
NHAMA;
CDR2:
(SEQ ID NO: 8)
YISYDGDNTYYRDSVKG;
and
CDR3:
(SEQ ID NO: 3)
GGNSGYD;

and the VL of said antibody or antigen-binding fragment thereof comprises the following CDRs:

CDR1:
(SEQ ID NO: 9)
RASQNVNKFLN;
CDR2:
(SEQ ID NO: 10)
GTNSLQS;
and
CDR3:
(SEQ ID NO: 6)
QQYTSWPWT.

In one embodiment, the VH of said antibody or antigen-binding fragment thereof comprises the following CDRs:

CDR1:
(SEQ ID NO: 7)
NHAMA;
CDR2:
(SEQ ID NO: 11)
YISYDGDNTYYADSVKG;
and
CDR3:
(SEQ ID NO: 3)
GGNSGYD;

and the VL of said antibody or antigen-binding fragment thereof comprises the following CDRs:

CDR1:
(SEQ ID NO: 9)
RASQNVNKFLN;
CDR2:
(SEQ ID NO: 10)
GTNSLQS;
and
CDR3:
(SEQ ID NO: 6)
QQYTSWPWT.

In one embodiment, the VH of said antibody or antigen-binding fragment thereof comprises the following CDRs:

CDR1:
(SEQ ID NO: 7)
NHAMA
CDR2:
(SEQ ID NO: 11)
YISYDGDNTYYADSVKG;
and
CDR3:
(SEQ ID NO: 3)
GGNSGYD;

and the VL of said antibody or antigen-binding fragment thereof comprises the following CDRs:

CDR1:
(SEQ ID NO: 12)
KGSQNVNKFLN;
CDR2:
(SEQ ID NO: 13)
GTNSLQT;
and
CDR3:
(SEQ ID NO: 6)
QQYTSWPWT.

In one embodiment, the VH of said antibody or antigen-binding fragment thereof comprises the following CDRs:

CDR1:
(SEQ ID NO: 14)
SHAMA;
CDR2:
(SEQ ID NO: 11)
YISYDGDNTYYADSVKG;
and
CDR3:
(SEQ ID NO: 3)
GGNSGYD

and the VL of said antibody or antigen-binding fragment thereof comprises the following CDRs:

CDR1:
(SEQ ID NO: 12)
KGSQNVNKFLN;
CDR2:
(SEQ ID NO: 13)
GTNSLQT;
and
CDR3:
(SEQ ID NO: 6)
QQYTSWPWT.

In one embodiment, the VH of said antibody or antigen-binding fragment thereof comprises the following CDRs:

CDR1:
(SEQ ID NO: 14)
SHAMA;
CDR2:
(SEQ ID NO: 11)
YISYDGDNTYYADSVKG;
and
CDR3:
(SEQ ID NO: 3)
GGNSGYD,

and the VL of said antibody or antigen-binding fragment thereof comprises the following CDRs:

CDR1:
(SEQ ID NO: 9)
RASQNVNKFLN;
CDR2:
(SEQ ID NO: 10)
GTNSLQS;
and
CDR3:
(SEQ ID NO: 6)
QQYTSWPWT.

In one embodiment, the isolated anti-human CD25 antibody or antigen-binding fragment thereof as described hereinabove is chimeric, humanized or human. In one embodiment, said antibody or antigen-binding fragment is monovalent.

In one embodiment, the isolated anti-human CD25 antibody or antigen-binding fragment thereof as described hereinabove mediates antibody dependent cellular cytotoxicity, complement dependent cytotoxicity and/or antibody-dependent phagocytosis.

In one embodiment, the isolated anti-human CD25 antibody or antigen-binding fragment thereof as described hereinabove is a bispecific antibody.

The present invention further relates to a fusion protein comprising the isolated anti-human CD25 antibody or the antigen-binding fragment thereof as described hereinabove.

The present invention further relates to a nucleic acid encoding the isolated anti-human CD25 antibody or antigen-binding fragment thereof, or the fusion protein, as described hereinabove.

The present invention further relates to a pharmaceutical composition comprising the isolated anti-human CD25 antibody or antigen-binding fragment thereof, or the fusion protein, as described hereinabove, and at least one pharmaceutically acceptable excipient.

The present invention further relates to the isolated anti-human CD25 antibody or antigen-binding fragment thereof, the fusion protein or the pharmaceutical composition, as described hereinabove, for use as a medicament.

The present invention further relates to the isolated anti-human CD25 antibody or antigen-binding fragment thereof, the fusion protein or the pharmaceutical composition, as described hereinabove, for use in treating a cancer or an infectious disease in a subject in need thereof.

The present invention further relates to a combination of an immunotherapy and an isolated anti-human CD25 antibody or antigen-binding fragment thereof, a fusion protein or a pharmaceutical composition, as described hereinabove, for use in treating a cancer or an infectious disease in a subject in need thereof.

### BRIEF DESCRIPTION OF THE DRAWINGS

FIG. 1 is a histogram showing the binding of anti-CD25 antibodies of the present invention (MAB02, MAB09, MAB10, MAB17, MAB18), a control antibody (huIgG1) or Basiliximab on a CD25 positive cell line.

FIG. 2 is a combination of a graph (A) and of a histogram (B) showing antibody-dependent cell-mediated cytotoxicity (ADCC) induced by anti-CD25 antibodies of the present invention, a control antibody (huIgG1) or Basiliximab. FIG. 2A shows the lysis of CD25 positive cells induced by the incubation with anti-CD25 antibodies of the present invention (MAB02, MAB09, MAB10, MAB17 and MAB18), a control antibody (huIgG1) or Basiliximab at different concentrations (10⁻⁷, 10⁻⁶, 10⁻⁵, 10⁻⁴, 10⁻¹, 10⁻², 10⁻¹, 1 or 10 μg/mL). FIG. 2B represents the lysis of CD25 positive cells induced by the incubation with anti-CD25 antibodies of the present invention (MAB02, MAB09, MAB10, MAB17, MAB18) at 1 μg/mL, as compared with a human IgG1 control antibody or Basiliximab. Data are represented as means±SEM.

FIG. 3 is a histogram showing the percentage of antibody dependent phagocytosis (ADCP) induced by anti-CD25 antibodies of the present invention (MAB02, MAB09, MAB10, MAB17, MAB18) at 10 μg/mL, as compared to a human IgG1 control antibody or Basiliximab. Data are represented as means±SEM.

FIG. 4 is a histogram showing the impact of anti-CD25 antibodies of the present invention (MAB02, MAB09, MAB10, MAB17, MAB18) at 1 μg/mL on IL-2 binding in vitro, as compared with a human IgG1 control antibody or Basiliximab.

FIG. 5 is a histogram showing the impact of anti-CD25 antibodies of the present invention (MAB02, MAB09, MAB10, MAB17, MAB18) on IL-2 induced effector T cell proliferation, as compared with a human IgG1 control antibody, Basiliximab, 7G7B6 and MA-251. ###p<0.001, ####p<0.0001 vs 7G7B6, § § § p<0.001, § § § § p<0.0001 vs MA-251; One-Way ANOVA.

FIG. 6 is a combination of two histograms showing the impact of anti-CD25 antibodies of the present invention on Treg cells depletion within the CD45+ lymphocyte population.

FIG. 6A represents the percentage of Treg cells depletion within the CD45+ lymphocyte population following incubation with a human IgG1 control antibody, anti-CD25 antibodies of the present invention (MAB02, MAB09, MAB10, MAB17, MAB18) or Basiliximab. FIG. 6B represent the percentage of Treg cells within the CD45+ lymphocyte population following incubation with a human IgG1 control antibody, anti-CD25 antibodies of the present invention (MAB02, MAB09, MAB10, MAB17, MAB18) or Basiliximab. ****p<0,0001 vs huIgG1; One-Way ANOVA

FIG. 7 is a combination of two histograms (A, B) showing the impact of anti-CD25 antibodies of the present invention on CD4+ effector T cells and CD8+ effector T cells depletion within the CD45+ lymphocyte population.

FIG. 7A represents the percentage of CD4+ effector T cells within the CD45+ lymphocyte population following incubation with a human IgG1 control antibody, anti-CD25 antibodies of the present invention (MAB02, MAB09, MAB10, MAB17, MAB18) or Basiliximab. FIG. 7B represents the percentage of CD8+ effector T cells within the CD45+ lymphocyte population following incubation with a human IgG1 control antibody, anti-CD25 antibodies of the present invention (MAB02, MAB09, MAB10, MAB17, MAB18) or Basiliximab.

### DEFINITIONS

In the present invention, the following terms have the following meanings:

“About”, preceding a figure encompasses plus or minus 10%, or less, of the value of said figure. It is to be understood that the value to which the term “about” refers is itself also specifically, and preferably, disclosed.

“Adnectins”, also known as monobodies, is well known in the art and refers to proteins designed to bind with high affinity and specificity to antigens. They belong to the class of molecules collectively called “antibody mimetics”.

“Alphabody” that may also be referred to as Cell-Penetrating Alphabodies, refers to a type of antibody mimetics consisting of small 10 kDa proteins engineered to bind to a variety of antigens. Alphabodies are able to reach and bind to intracellular protein targets.

“Affibodies” refer to affinity proteins based on a 58 amino acid residue protein domain, derived from one of the IgG binding domain of staphylococcal protein A (Frejd & Kim, 2017. Exp Mol Med. 49(3):e306; patent U.S. Pat. No. 5,831,012).

“Affilins” refer to artificial proteins designed to selectively bind antigens. They resemble antibodies in their affinity and specificity to antigens but not in structure which makes them a type of antibody mimetic.

“Affinity” and “avidity” are used to defined the strength of an antibody-antigen complex. Affinity measures the strength of interaction between an epitope and an antigen binding site on an antibody. It may be expressed by an affinity constant K_{A} or by a dissociation constant K_{D}. Avidity (or functional affinity) gives a measure of the overall strength of an antibody-antigen complex. It may depend on different parameters, including in particular the affinity of the antibody or antigen-binding fragment thereof for an epitope, (ii) the valency of both the antibody and the antigen and (iii) structural arrangement of the parts that interact.

“Antibody” and “immunoglobulin”, as used herein, may be used interchangeably and refer to a protein having a combination of two heavy and two light chains whether or not it possesses any relevant specific immunoreactivity. “Antibodies” refers to such assemblies which have significant known specific immunoreactive activity to an antigen of interest (e.g., human CD25). The term “anti-hCD25 antibodies” is used herein to refer to antibodies which exhibit immunological specificity for human CD25 protein. As explained elsewhere herein, “specificity” for human CD25 (hCD25) does not exclude cross-reaction with species homologues of hCD25, such as, for example, with simian CD25. Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (VL domain) and heavy chain variable domain (VH domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the VH and VL chains.

“Affitins” refer to highly stable engineered affinity proteins, originally derived from Sac7d and Sso7d, two 7 kDa DNA-binding polypeptides from Sulfolobus genera.

“Anticalins” refer to an antibody mimetic technology, wherein the binding specificity is derived from lipocalins. Anticalins may also be formatted as dual targeting protein, called Duocalins.

“Antigen-binding fragment”, as used herein, refers to a part or region of an antibody which comprises fewer amino acid residues than the whole antibody. An “antigen-binding fragment” binds antigen and/or competes with the whole antibody from which it derives for antigen binding (e.g., specific binding to human CD25). Antibody antigen-binding fragments encompasses, without any limitation, single chain antibodies, Fv, Fab, Fab′, Fab′-SH, F(ab)′₂, Fd, defucosylated antibodies, diabodies, triabodies and tetrabodies.

“Armadillo repeat protein-based scaffold”, as used herein, refers to a type of antibody mimetics corresponding to artificial peptide binding scaffolds based on armadillo repeat proteins. Armadillo repeat proteins are characterized by an armadillo domain, composed of tandem armadillo repeats of approximately 42 amino acids, which mediates interactions with peptides or proteins.

“Atrimers” refer to binding molecules for target protein that trimerize as a perquisite for their biological activity. They are relatively large compared to other antibody mimetic scaffolds.

“Avimers” refer to an antibody mimetic technology.

“CD25” refers to any native CD25 from any vertebrate source, including mammals such as primates (e.g., humans) and rodents (e.g., mice and rats), unless otherwise indicated. The Interleukin-2 receptor alpha chain (also called CD25) protein is encoded by the IL2RA gene. Two forms of the IL-2 receptor were described: the first one comprising the alpha subunit (CD25), the beta subunit (CD122) and the gamma subunit (CD132), and the second one comprising only the beta and gamma subunits (i.e., CD122 and CD132). The term encompasses “full-length” or unprocessed CD25 as well as any form of CD25 that results from processing in the cell. The term also encompasses naturally occurring variants of CD25 (e.g., splice variants or allelic variants). In certain embodiments CD25 is human CD25. For example, CD25 is expressed by activated T lymphocytes and activated B lymphocytes responding to antigen or mitogen stimulation. CD25 is also expressed by regulatory T cells (CD25^{high} FoxP3⁺ regulatory T cells). In one embodiment, CD25 refers to human CD25 (Uniprot accession number P01589, SEQ ID NO: 39).

SEQ ID NO: 39
MDSYLLMWGLLTFIMVPGCQAELCDDDPPEIPHATFKAMA
YKEGTMLNCECKRGFRRIKSGSLYMLCTGNSSHSSWDNQC
QCTSSATRNTTKQVTPQPEEQKERKTTEMOSPMQPVDQAS
LPGHCREPPPWENEATERIYHFVVGQMVYYQCVQGYRALH
RGPAESVCKMTHGKTRWTQPQLICTGEMETSQFPGEEKPQ
ASPEGRPESETSCLVTTTDFQIQTEMAATMETSIFTTEYQ
VAVAGCVFLLISVLLLSGLTWQRRQRKSRRTI

“CDR” or “complementarity determining region” means the non-contiguous antigen combining sites found within the variable region of both heavy and light chain polypeptides. The precise amino acid sequence boundaries of a given CDR can be determined using any of a number of well-known schemes, including those described by Kabat et al. (1991), “Sequences of Proteins of Immunological Interest” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof. More recently, a universal numbering system has been developed and widely adopted, ImMunoGeneTics (IMGT) Information System® (Lefranc et al., Nucleic Acids Res. 27: 209-212 1999). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues may be readily identified. This information can be used in grafting and replacement of CDR residues from immunoglobulins of one species into an acceptor framework from, typically, a human antibody. Correspondence between the Kabat numbering and the IMGT unique numbering system is also well known to one skilled in the art (e.g., Lefranc et al., supra). Thus, in one embodiment, by CDR regions or CDR, it is intended to indicate the hypervariable regions of the heavy and light chains of the immunoglobulins as defined by IMGT® numbering system (e.g. Lefranc et al., supra).

“DARPins” (Designed Ankyrin Repeat Proteins) refer to an antibody mimetic DRP (designed repeat protein) technology developed to exploit the binding abilities of non-antibody polypeptides.

“Diabodies”, as used herein, refer to small antibody fragments prepared by constructing scFv fragments with short linkers (about 5-10 residues) between the VH and VL such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the VH and VL of the two antibodies are present on different polypeptide chains. Diabodies are described, for example, in patent EP0404097 or patent application WO1993011161.

“Domain antibodies” refer to the smallest functional binding units of antibodies, corresponding to the variable regions of either the heavy or light chains of antibodies.

“Domain kunitz peptide” refer to a type of antibody mimetics, and is based on the active domains of proteins inhibiting the function of proteases.

“Effector T cells” refer to a group of cells that includes several T cell types (e.g., CD4+ and CD8+ T cells). It includes helpers T cells (Th cells) that help other leukocytes in immunologic processes, including maturation of B cells into plasma cells and memory B cells and cytotoxic T cells (Tc cells, CTLs, T-killer cells, killer T cells) that destroy virus-infected cells and tumor cells, and are also implicated in transplant rejection.

“Epitope” refers to a specific arrangement of amino acids located on a protein or proteins to which an antibody or antigen-binding fragment thereof or an antibody mimetic binds.

Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Evasins” refer to a class of chemokine-binding proteins.

“Fab” refers to a monovalent fragment containing the following regions: VH, VL, CH1 and CL, linked by an intramolecular disulfide bond. As used herein, F(ab′)₂ refers to a fragment containing two antigen-binding regions joined by disulfides bonds. As used herein, Fab′ refers to a fragment obtained by the reduction of F(ab′)2 fragments.

“Framework region” or “FR region” includes the amino acid residues that are part of the variable region, but are not part of the CDRs (e.g., using the IMGT® numbering definition of CDRs). The framework regions for the light chain are similarly separated by each of the VL's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a n-sheet conformation and the CDRs form loops which connect, and in some cases form part of, the n-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof.

“Fd fragment” refers to the heavy chain of the Fab fragment, comprising the VH and CH1 regions.

“Fynomers” refer to proteins that belong to the class of antibody mimetic. They are attractive binding molecules due to their high thermal stability and reduced immunogenicity.

“Fv”, as used herein, refers to the minimum antibody fragment that contains a complete antigen-recognition and -binding site. This fragment consists of a dimer of one VH and one VL in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Heavy chain region” includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or antigen-binding fragment thereof according to the present invention may comprise the Fe region of an immunoglobulin heavy chain (e.g., a hinge portion, a C_{H}2 domain, and a C_{H}3 domain). In another embodiment, the antibody or antigen-binding fragment thereof according to the present invention lacks at least a region of a constant domain (e.g., all or part of a C_{H}2 domain). In certain embodiments, at least one, and preferably all, of the constant domains are derived from a human immunoglobulin heavy chain. For example, in one embodiment, the heavy chain region comprises a fully human hinge domain. In other embodiments, the heavy chain region comprises a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. In some embodiments, the constant domains of the heavy chain region may be modified such that they vary in amino acid sequence from the naturally occurring (wild-type) immunoglobulin molecule. That is, the antibody or antigen-binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

“Hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90).

“Hypervariable loop” is a term not strictly synonymous to complementarity determining region (CDR), since the hypervariable loops (HVs) are defined on the basis of structure, whereas CDRs are defined based on sequence variability (Kabat et al., 1991. Sequences of proteins of immunological interest (5^{th} ed.). Bethesda, MD: U.S. Dep. of Health and Human Services) and the limits of the HVs and the CDRs may be different in some VH and V_{L} domains.

“Identity” or “identical”, when used herein in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Such methods include, but are not limited to, those described in Lesk A. M. (1988). Computational molecular biology: Sources and methods for sequence analysis. New York, NY: Oxford University Press; Smith D. W. (1993). Biocomputing: Informatics and genome projects. San Diego, CA: Academic Press; Griffin A. M. & Griffin H. G. (1994). Computer analysis of sequence data, Part 1. Totowa, NJ: Humana Press; von Heijne G. (1987). Sequence analysis in molecular biology: treasure trove or trivial pursuit. San Diego, CA: Academic press; Gribskov M. R. & Devereux J. (1991). Sequence analysis primer. New York, NY: Stockton Press; Carillo et al., 1988. SIAM J Appl Math. 48(5):1073-82. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Interleukin-2” or “IL-2”, as used herein, refers to any native IL-2 from any vertebrate source, including mammals such as primates (e.g., humans) and rodents (e.g., mice and rats), unless otherwise indicated. The term encompasses unprocessed IL-2 as well as any form of IL-2 that results from processing in the cell. The term also encompasses naturally occurring variants of IL-2 (e.g., splice variants or allelic variants). In one embodiment, IL-2 is human IL-2, having the sequence SEQ ID NO: 40.

SEQ ID NO: 40
MYRMQLLSCIALSLALVTNSAPTSSSTKKTQLQLEHLLLD
LQMILNGINNYKNPKLTRMLTFKFYMPKKATELKHLQCLE
EELKPLEEVLNLAQSKNFHLRPRDLISNINVIVLELKGSE
TTFMCEYADETATIVEFLNRWITFCQSIISTLT

“Knottin” (that may also be referred to as inhibitor cystine knot) refers to an antibody mimetic comprising a protein structural motif containing three disulfide bridges.

“Mammal” refers to any mammal, including humans, non-human primates, domestic and farm animals, and zoo, sports, or pet animals, such as dogs, cats, cattle, horses, sheep, pigs, goats, rabbits, monkeys, etc. Preferably, the mammal is human.

“Monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or antigen-binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (patent U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

“Nanobodies” refer to antibody-derived therapeutic proteins that contain the unique structural and functional properties of naturally-occurring heavy chain antibodies (Muyldermans, 2013. Annu Rev Biochem. 82:775-97). These heavy chain antibodies may contain a single variable domain (VHH) and two constant domains (C_{H}2 and C_{H}3).

“Prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

“Regulatory T cell” or “Treg cell” refers to a specialized type of T cells, in particular of CD4⁺ T cell, that can suppress the responses of other T cells. Treg cells are generally characterized by expression of CD4, the α-subunit of the IL-2 receptor (CD25), and the transcription factor forkhead box P3 (Foxp3) and play a critical role in the induction and maintenance of peripheral self-tolerance to antigens, including those expressed by tumors. More recently, CD8 Tregs have also been described.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the VH and V_{L} antibody domains connected into a single amino acid chain. Preferably, the scFv amino acid sequence further comprises a peptide linker between the VH and V_{L} domains that enables the scFv to form the desired structure for antigen binding.

“Subject”, as used herein, refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease.

“Therapeutically effective amount” refers to the level or amount of an antibody as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. In one embodiment, a subject is successfully “treated” for a cancer or an infectious disease if, after receiving a therapeutic amount of an antibody according to the present invention, the subject shows at least one of the following: reduction in the number of cancer cells (or tumor size) or pathogenic cells; reduction in the percent of total cells that are cancerous or pathogenic; relief to some extent of one or more of the symptoms associated with the cancer or the infectious disease to be treated; reduced morbidity and mortality; and improvement in quality of life issues. The above parameters for assessing successful treatment and improvement in the disease are readily measurable by routine procedures familiar to a physician.

“Tumor infiltrating Tregs” relates to CD25^{+/hi} Foxp3⁺ regulatory T cells that accumulate within neoplastic lesions as a result of several distinct mechanisms, including increased infiltration, local expansion, survival advantage and in situ development from conventional CD4⁺ or CD8⁺ cells.

“Unibodies” refer to an antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies.

“Variable” refers to the fact that certain regions of the variable domains VH and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the VH domain which form part of the antigen binding site. The first, second and third hypervariable loops of the Vλ light chain domain are referred to herein as L1 (λ), L2 (λ) and L3 (λ) and may be defined as comprising residues 24-33 (L1 (λ), consisting of 9, 10 or 11 amino acid residues), 49-53 L2 (λ), consisting of 3 residues) and 90-96 (L3 (λ), consisting of 6 residues) in the V_{L} domain (Morea et al., 2000. Methods. 20(3):267-79). The first, second and third hypervariable loops of the Vκ light chain domain are referred to herein as L1 (κ), L2 (κ) and L3 (κ) and may be defined as comprising residues 25-33 (L1 (κ), consisting of 6, 7, 8, 11, 12 or 13 residues), 49-53 (L2 (κ), consisting of 3 residues) and 90-97 (L3 (κ), consisting of 6 residues) in the V_{L} domain (Morea et al., supra). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., supra). Unless otherwise indicated, the terms L1, L2 and L3 respectively refer to the first, second and third hypervariable loops of a VL domain, and encompass hypervariable loops obtained from both Vκ and VW isotypes. The terms H1, H2 and H3 respectively refer to the first, second and third hypervariable loops of the VH domain, and encompass hypervariable loops obtained from any of the known heavy chain isotypes, including gamma (γ), mu (μ), alpha (α), delta (δ) or epsilon (ε). The hypervariable loops L1, L2, L3, H1, H2 and H3 may each comprise part of a “complementarity determining region” or “CDR”, as defined hereinabove.

“Versabodies” refer to an antibody mimetic technology. They are small proteins of 3-5 kDa with >15% cysteines, which form a high disulfide density scaffold, replacing the hydrophobic core the typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

### DETAILED DESCRIPTION

Different studies have analyzed the interaction between IL-2 and CD25 in murine models. It has been shown that the blockade of the binding of IL-2 to CD25, with an anti-CD25 antibody, such as, for example PC61, in tumor-bearing mice results in a loss of both FoxP3 expression and Tregs suppressive function. This result suggests that the blockade of IL2 binding to CD25 represents a promising method for preventing cancer development. Recently, Vargas et al. (2017, Immunity 48(6), 577-586) have developed an Fc optimized form of the PC61 antibody allowing intra-tumoral Tregs depletion via antibody-dependent cellular cytotoxicity (ADCC) or complement-mediated cytotoxicity mechanisms (CDC) offering a significant therapeutic benefit in murine tumor models. Given the physiological importance of CD25 and of the IL-2 pathway in Tregs, the blockade of said pathway thus seemed to be a powerful and promising antitumoral immunotherapy.

Despite the apparent promise of IL-2 pathway blockade as an antitumoral immunotherapy, the manipulation of the IL-2 pathway should be carefully examined as it modulates both immuno-stimulatory and immuno-regulatory functions. Indeed, while the IL-2 pathway plays an important role in regulating immune responses and maintaining peripheral self-tolerance, it also acts as a T cell growth factor, essential for the proliferation and survival of T cells as well as for the generation of effector and memory T cells. Furthermore, IL-2 receptors are also transiently expressed in effector T cells and myeloid dendritic cells, and therefore IL-2 pathway manipulation could cause unpredicted outcomes, such as, for example, an alteration of antitumor effector T cells, in particular of CD8+ effector T cells, function, resulting in cancer progression.

As a component of the immune system, effector CD8+ T cells have important roles in suppressing tumors. For example, effector CD8+ T cells can kill tumor cells with cytotoxic molecules, such as granzymes and perforin. IFN-γ, which is produced by CD8+ T cells, can increase the expression of MHC class I antigens by tumor cells, thereby rendering them better targets for CD8+ T cells. Thus, during cancer, effector CD8+ T cells are critical for the elimination of neoplastic cells.

Here, the Applicants aimed to eliminate or silence Tregs while maintaining an efficient effector T cells response during cancer. Thus, the present invention relates to novel anti-CD25 antibodies (in particular anti-human CD25 antibodies) that exhibit a potent anti-cancer effect, in particular by depleting Tregs, without blocking of the IL-2 signaling pathway, thereby allowing IL-2 to stimulate effector T cells.

The present invention thus first relates to an isolated protein which binds to human CD25 (hCD25).

In one embodiment, the isolated protein according to the present invention is an isolated antibody or antigen-binding fragment thereof, wherein said antibody or antigen-binding fragment thereof binds to human CD25 (hCD25).

An “isolated protein”, and in particular an “isolated antibody”, as used herein, is intended to refer to a protein, in particular an antibody that is substantially free of other proteins or antibodies having different antigenic specificities (e.g., an isolated protein or antibody that specifically binds hCD25 is substantially free of proteins or antibodies that specifically bind antigens other than hCD25). An isolated protein, in particular an isolated antibody, that specifically binds hCD25 may, however, have cross-reactivity to other antigens, such as hCD25 molecules from other species. Moreover, an isolated protein or antibody may be substantially free of other cellular material and/or chemicals, in particular those that would interfere with therapeutic uses of the protein or antibody, including without limitation, enzymes, hormones, and other proteinaceous or non-proteinaceous components.

In one embodiment, the isolated protein, in particular the isolated antibody or antigen-binding fragment thereof is purified.

In one embodiment, the isolated protein or antibody (or antigen-binding fragment thereof) is purified to:
(1) greater than 80%, 85%, 90%, 91%, 92%, 93%, 94%, 95% or more by weight of protein or of antibody (or antigen-binding fragment thereof) as determined by the Lowry method, and most preferably greater than 96%, 97%, 98% or 99% by weight;
(2) a degree sufficient to obtain at least 15 residues of N-terminal or internal amino acid sequence by use of a spinning cup sequenator; or
(3) homogeneity as shown by SDS-PAGE under reducing or non-reducing conditions and using Coomassie blue or, preferably, silver staining.

According to the present invention, the isolated protein, in particular the isolated antibody or antigen-binding fragment thereof does not inhibit the signaling of IL-2 via CD25. In one embodiment, the isolated protein does not inhibit the binding of interleukin-2 (IL-2) to human CD25. In one embodiment, the isolated antibody or antigen-binding fragment thereof does not inhibit the binding of interleukin-2 (IL-2) to human CD25, and may thus be referred herein as a “non-blocking antibody”.

In one embodiment, the protein, the antibody or the antigen-binding fragment of the antibody according to the present invention inhibits less than 50% of the IL-2 signaling compared to IL-2 signaling in the absence of the protein, antibody or antigen-binding fragment of the antibody. In one embodiment, the protein, the antibody or the antigen-binding fragment of the antibody according to the present invention inhibits less than 45%, 40%, 35%, 30%, 25%, 20%, 15%, 10%, 5% of the IL-2 signaling compared to IL-2 signaling in the absence of the protein, antibody or antigen-binding fragment of the antibody.

Methods for measuring the IL-2 signaling are well known in the art and comprise, for example, the measurement of the induction of IL-2 receptor signaling (e.g., by detection of phosphorylated STAT5a), the measurement of the induction of T cell proliferation (e.g., by detection of Ki-67 using in particular CellTrace™ Cell Proliferation Kits, by direct assessment of T cell proliferation in the presence of IL-2, in MLR experiments (comprising, for example, the activation of cells with CD3 and CD28 in the presence of IL-2), or using cell lines that depend on IL-2 to proliferate, such as, for example CTLL2 cell line) and/or the measurement of an up-regulation of expression of activation markers (such as e.g., CD25, CD69, cytotoxic molecules, such as, for example, granzyme B, and the like).

In one embodiment, the protein, the antibody or the antigen-binding fragment of the antibody of the present invention does not inhibit the proliferation and/or activation of CD4⁺ and CD8⁺ T cells. In one embodiment, the protein, the antibody or the antigen-binding fragment of the antibody of the present invention does not inhibit the IL-2 induced proliferation of CD4⁺ and CD8⁺ T cells (an example of a method that may be used for measuring IL-2 induced proliferation of T cells is provided in the Example part).

In one embodiment, the protein, the antibody or the antigen-binding fragment of the antibody of the present invention inhibits the IL-2 induced proliferation of CD4⁺ and CD8⁺ T cells by less than 30%, preferably less than 25%, 20%, 15%, 10% or less as compared to the IL-2 induced proliferation of CD4⁺ and CD8⁺ T cells using an isotype control antibody.

In one embodiment, the protein, the antibody or the antigen-binding fragment of the antibody according to the present invention does not inhibit the phosphorylation of STAT5a in CD4⁺ and CD8⁺ T cells.

In one embodiment, the protein, the antibody or the antigen-binding fragment of the antibody according to the present invention inhibits less than 50% of the IL-2 binding to CD25 as compared to IL-2 binding to CD25 in the absence of the protein, antibody or antigen-binding fragment respectively. In one embodiment, the protein, the antibody or the antigen-binding fragment of the antibody according to the present invention inhibits less than 45%, 40%, 35%, 30%, 25%, 20%, 15%, 10%, 5% of the IL-2 binding to CD25 as compared to IL-2 binding to CD25 in the absence of the protein, antibody or antigen-binding fragment respectively.

Examples of methods for measuring the IL-2 binding to CD25 are well known from the skilled artisan and include, without limitation, detection of a labeled-IL-2 on CD25, such as, for example, of a biotinylated or radiolabeled IL-2 on CD25.

In one embodiment, the protein, the antibody or the antigen-binding fragment of the antibody according to the present invention is specific for human CD25 (hCD25).

A protein, antibody or antigen-binding fragment thereof is said to be “specific for”, “immunospecific” or to “specifically bind” an antigen if it reacts at a detectable level with said antigen (e.g., CD25), preferably with an affinity constant (K_{A}) of greater than or equal to about 10⁶ M⁻¹, preferably greater than or equal to about 10⁷ M⁻¹, 5×10⁸ M⁻¹, 5×10⁸ M⁻¹, 10⁹ M⁻¹, 5×10⁹ M⁻¹ or more. Affinity of a protein, or of an antibody or antigen-binding fragment thereof for its cognate antigen is also commonly expressed as an equilibrium dissociation constant (K_{D}). An antibody or antigen-binding fragment thereof is said to be “immunospecific”, “specific for” or to “specifically bind” an antigen if it reacts at a detectable level with said antigen (e.g., CD25), preferably with a K_{D} of less than or equal to 10⁻⁶ M, preferably less than or equal to 10⁻⁷ M, 5.10⁻⁸ M, 10⁻⁸ M, 5.10⁻⁹ M, 10⁻⁹ M or less.

Affinities of antibodies or antigen-binding fragment thereof can be readily determined using conventional techniques, for example, those described by Scatchard, 1949. Ann NY Acad Sci. 51:660-672. Binding properties of an antibody or antigen-binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®).

In one embodiment, the protein (in particular the antibody or antigen-binding fragment thereof) according to the present invention presents a K_{D} for binding to human CD25 inferior or equal to about 50.10⁻⁹ M, preferably inferior or equal to about 40.10⁻⁹ M or to about 30.10⁻⁹ M. In one embodiment, the K_{D} of the protein of the invention for binding to human CD25 ranges from about 1.10⁻⁹ M to about 50.10⁻⁹ M, preferably from about 2.10⁻⁹ M to about 30.10⁻⁹ M.

In one embodiment, the protein, antibody or antigen-binding fragment thereof according to the present invention is polyclonal.

In another embodiment, the protein, antibody or antigen-binding fragment thereof according to the present invention is monoclonal.

In one embodiment, the antibody or antigen-binding fragment thereof according to the present invention is a molecule selected from the group comprising or consisting of a whole antibody, a humanized antibody, a single chain antibody, a dimeric single chain antibody, a Fv, a Fab, a Fab′, a Fab′-SH, a F(ab)′₂, a Fd, a defucosylated antibody, a bispecific antibody, a diabody, a triabody and a tetrabody.

In one embodiment, the antibody or antigen-binding fragment thereof according to the present invention is a molecule selected from the group comprising or consisting of a whole antibody, a single chain variable fragment (scFv), a Fv, a Fab, a Fab′, a Fab′-SH, a F(ab)′₂, a defucosylated antibody, a bispecific antibody, a diabody, a triabody and a tetrabody.

Antigen-binding fragments of antibodies can be obtained using standard methods. For instance, Fab or F(ab′)₂ fragments may be produced by protease digestion of the isolated antibodies, according to conventional techniques.

It will also be appreciated that proteins, antibodies or antigen-binding fragments thereof according to the present invention can be modified using known methods. For example, to slow clearance in vivo and obtain a more desirable pharmacokinetic profile, the protein, antibody or antigen-binding fragment thereof may be modified with polyethylene glycol (PEG). Methods for coupling and site-specifically conjugating PEG to an antibody or antigen-binding fragment thereof are described in, e.g., Leong et al., 2001. Cytokine. 16(3):106-19; Delgado et al., 1996. Br J Cancer. 73(2):175-82.

In one embodiment, the antibody or antigen-binding fragment thereof according to the present invention is a molecule selected from the group comprising or consisting of a unibody, a domain antibody, and a nanobody. In one embodiment, the antibody or antigen-binding fragment thereof according to the present invention is a unibody.

In one embodiment, the isolated protein according to the present invention is an antibody mimetic selected from the group comprising or consisting of an affibody, an alphabody, an armadillo repeat protein-based scaffold, a knottin, a domain kunitz peptide, an affilin, an affitin, an adnectin, an atrimer, an evasin, a DARPin, an anticalin, an avimer, a fynomer, a versabody or a duocalin.

In the following, and unless explicitly mentioned otherwise, CDR numbering and definitions are according to the IMGT® numbering system.

In one embodiment, the antibody or antigen-binding fragment thereof according to the present invention comprises a heavy chain variable region (abbreviated herein as VH) which comprises at least one, preferably at least two, more preferably the three following complementary-determining regions (CDRs):

CDR1:
(SEQ ID NO: 1)
X₁HAMA,
wherein X₁ is N or S;
CDR2:
(SEQ ID NO: 2)
YISYDGDNTYYX₂DSVKG
wherein X₂ is R or A;
and/or
CDR3:
(SEQ ID NO: 3)
GGNSGYD.

In one embodiment, the antibody or antigen-binding fragment thereof according to the present invention comprises a VH which comprises the three following CDRs:

CDR1:
(SEQ ID NO: 1)
X₁HAMA,
wherein X₁ is N or S;
CDR2:
(SEQ ID NO: 2)
YISYDGDNTYYX₂DSVKG
wherein X₂ is R or A;
and
CDR3:
(SEQ ID NO: 3)
GGNSGYD.

In one embodiment, the antibody or antigen-binding fragment thereof of the present invention comprises a VH which comprises at least one, preferably at least two, more preferably the three following CDRs:

CDR1:
(SEQ ID NO: 1)
X₁HAMA, wherein X₁ is N or S;
CDR2:
(SEQ ID NO: 11)
YISYDGDNTYYADSVKG; and/or
CDR3:
(SEQ ID NO: 3)
GGNSGYD.

In one embodiment, the antibody or antigen-binding fragment thereof according to the present invention comprises a VH which comprises the three following CDRs:

CDR1:
(SEQ ID NO: 1)
X₁HAMA,
wherein X₁ is N or S;
CDR2:
(SEQ ID NO: 11)
YISYDGDNTYYADSVKG;
and
CDR3:
(SEQ ID NO: 3)
GGNSGYD.

In one embodiment, the antibody or antigen-binding fragment thereof according to the present invention comprises a VH which comprises at least one (e.g., 1, 2 or 3) of the following CDR, and preferably the three following CDRs:

CDR1:
(SEQ ID NO: 7)
NHAMA;
CDR2:
(SEQ ID NO: 8)
YISYDGDNTYYRDSVKG;
and
CDR3:
(SEQ ID NO: 3)
GGNSGYD.

In one embodiment, the antibody or antigen-binding fragment thereof according to the present invention comprises a VH which comprises at least one (e.g., 1, 2 or 3) of the following CDR, and preferably the three following CDRs:

CDR1:
(SEQ ID NO: 7)
NHAMA;
CDR2:
(SEQ ID NO: 11)
YISYDGDNTYYADSVKG;
and
CDR3:
(SEQ ID NO: 3)
GGNSGYD.

In one embodiment, the antibody or antigen-binding fragment thereof according to the present invention comprises a VH which comprises at least one (e.g., 1, 2 or 3) of the following CDR, and preferably the three following CDRs:

CDR1:
(SEQ ID NO: 14)
SHAMA;
CDR2:
(SEQ ID NO: 11)
YISYDGDNTYYADSVKG;
and
CDR3:
(SEQ ID NO: 3)
GGNSGYD.

In one embodiment, the antibody or antigen-binding fragment thereof according to the present invention comprises a VH which comprises at least one (e.g., 1, 2 or 3) of the following CDR, and preferably the three following CDRs:

CDR1:
(SEQ ID NO: 14)
SHAMA;
CDR2:
(SEQ ID NO: 8)
YISYDGDNTYYRDSVKG;
and
CDR3:
(SEQ ID NO: 3)
GGNSGYD.

In one embodiment, any of CDR1, CDR2 and/or CDR3 of the VH with SEQ ID NOs 1-3, 7, 8, 11 or 14 can be characterized as having 1, 2, 3 or more amino acids being substituted by a different amino acid.

In one embodiment, any of CDR1, CDR2 and/or CDR3 of the VH with SEQ ID NOs 1-3, 7, 8, 11 or 14 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

In one embodiment, the antibody or antigen-binding fragment thereof according to the present invention comprises a light chain variable region (abbreviated herein as VL) which comprises at least one, preferably at least two, more preferably the three following complementary-determining regions (CDRs):

CDR1:
(SEQ ID NO: 4)
X₃X₄SQNVNKFLN
wherein X₃ is K or R and wherein
X₄ is G or A;
CDR2:
(SEQ ID NO: 5)
GTNSLQX₅
wherein X₅ is T or S;
and/or
CDR3:
(SEQ ID NO: 6)
QQYTSWPWT.

In one embodiment, the antibody or antigen-binding fragment thereof according to the present invention comprises a VL which comprises the three following CDRs:

CDR1:
(SEQ ID NO: 4)
X₃X₄SQNVNKFLN
wherein X₃ is K or R and wherein
X₄ is G or A;
CDR2:
(SEQ ID NO: 5)
GTNSLQX₅ wherein X₅ is T or S;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 9)
RASQNVNKFLN;
CDR2:
(SEQ ID NO: 10)
GTNSLQS;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 12)
KGSQNVNKFLN;
CDR2:
(SEQ ID NO: 13)
GTNSLQT;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 9)
RASQNVNKFLN;
CDR2:
(SEQ ID NO: 13)
GTNSLQT;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 12)
KGSQNVNKFLN;
CDR2:
(SEQ ID NO: 10)
GTNSLQS;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 15)
RGSQNVNKFLN;
CDR2:
(SEQ ID NO: 13)
GTNSLQT;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 15)
RGSQNVNKFLN;
CDR2:
(SEQ ID NO: 10)
GTNSLQS;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 16)
KASQNVNKFLN;
CDR2:
(SEQ ID NO: 13)
GTNSLQT;
and
CDR3:
(SEQ ID NO: 6)
QQYTSWPWT.

In one embodiment, the antibody or antigen-binding fragment thereof according to the present invention comprises a VL which comprises at least one (e.g., 1, 2 or 3) of the following CDR, and preferably the three following CDRs:

CDR1:
(SEQ ID NO: 16)
KASQNVNKFLN;
CDR2:
(SEQ ID NO: 10)
GTNSLQS;
and
CDR3:
(SEQ ID NO: 6)
QQYTSWPWT.

In one embodiment, any of CDR1, CDR2 and/or CDR3 of the VL with SEQ ID NOs 4-6, 9, 10, 12, 13, 15 or 16 can be characterized as having 1, 2, 3, 4, 5 or more amino acids being substituted by a different amino acid.

In one embodiment, any of CDR1, CDR2 and/or CDR3 of the VL with SEQ ID NOs 4-6, 9, 10, 12, 13, 15 or 16 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

In one embodiment, the antibody or antigen-binding fragment thereof of the present invention comprises:
a VH which comprises at least one, preferably at least two, more preferably the three following CDRs:
   CDR1: X₁HAMA (SEQ ID NO: 1), wherein X₁ is N or S;
   CDR2: YISYDGDNTYYX₂DSVKG (SEQ ID NO: 2) wherein X₂ is R or A; and/or
   CDR3: GGNSGYD (SEQ ID NO: 3); and
a VL which comprises at least one, preferably at least two, more preferably the three following CDRs:
   CDR1: X₃X₄SQNVNKFLN (SEQ ID NO: 4) wherein X₃ is K or R and wherein X₄ is G or A;
   CDR2: GTNSLQX₅(SEQ ID NO: 5) wherein X₅ is T or S; and/or
   CDR3: QQYTSWPWT (SEQ ID NO: 6), and
with the proviso that
when X₁ is N and X₂ is R, X₃ is R and/or X₅ is S; and
when X₃ is K and X₅ is T, X₁ is S and/or X₂ is A.

In one embodiment, the antibody or antigen-binding fragment thereof of the present invention comprises:
a VH comprising the three following CDRs:
   CDR1: X₁HAMA (SEQ ID NO: 1), wherein X₁ is N or S;
   CDR2: YISYDGDNTYYX₂DSVKG (SEQ ID NO: 2) wherein X₂ is R or A; and
   CDR3: GGNSGYD (SEQ ID NO: 3); and
a VL comprising the three following CDRs:
   CDR1: X₃X₄SQNVNKFLN (SEQ ID NO: 4) wherein X₃ is K or R and wherein X₄ is G or A;
   CDR2: GTNSLQX₅(SEQ ID NO: 5) wherein X₅ is T or S; and
   CDR3: QQYTSWPWT (SEQ ID NO: 6), and
with the proviso that
when X₁ is N and X₂ is R, X₃ is R and/or X₅ is S; and
when X₃ is K and X₅ is T, X₁ is S and/or X₂ is A.

In one embodiment, any of CDR1, CDR2 and/or CDR3 of the VH with SEQ ID NOs 1-3 and/or of the VL with SEQ ID NOs 4-6 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

In one embodiment, the antibody or antigen-binding fragment thereof of the present invention comprises:
a VH which comprises at least one, preferably at least two, more preferably the three following CDRs:
   CDR1: X₁HAMA (SEQ ID NO: 1), wherein X₁ is N or S;
   CDR2: YISYDGDNTYYADSVKG (SEQ ID NO: 11); and/or
   CDR3: GGNSGYD (SEQ ID NO: 3); and
a VL which comprises at least one, preferably at least two, more preferably the three following CDRs:

CDR1:
(SEQ ID NO: 9)
RASQNVNKFLN;
CDR2:
(SEQ ID NO: 10)
GTNSLQS;
and/or
CDR3:
(SEQ ID NO: 6)
QQYTSWPWT.

In one embodiment, the antibody or antigen-binding fragment thereof of the present invention comprises:
a VH comprising the three following CDRs:
   CDR1: X₁HAMA (SEQ ID NO: 1), wherein X₁ is N or S;
   CDR2: YISYDGDNTYYADSVKG (SEQ ID NO: 11); and
   CDR3: GGNSGYD (SEQ ID NO: 3); and
a VL comprising the three following CDRs:
   CDR1: RASQNVNKFLN (SEQ ID NO: 9);
   CDR2: GTNSLQS (SEQ ID NO: 10); and
   CDR3: QQYTSWPWT (SEQ ID NO: 6).

In one embodiment, the antibody or antigen-binding fragment thereof of the present invention comprises:
a VH comprising the three following CDRs:
   CDR1: NHAMA (SEQ ID NO: 7);
   CDR2: YISYDGDNTYYRDSVKG (SEQ ID NO: 8); and
   CDR3: GGNSGYD (SEQ ID NO: 3); and
a VL comprising the three following CDRs:
   CDR1: RASQNVNKFLN (SEQ ID NO: 9);
   CDR2: GTNSLQS (SEQ ID NO: 10); and
   CDR3: QQYTSWPWT (SEQ ID NO: 6).

In one embodiment, any of CDR1, CDR2 and/or CDR3 of the VH with SEQ ID NOs 3, 7-8 and/or of the VL with SEQ ID NOs 6, 9-10 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

Examples of antibodies comprising a heavy chain comprising CDR1, CDR2 and CDR3 with SEQ ID NOs 7, 8 and 3 and a light chain comprising CDR1, CDR2 and CDR3 with SEQ ID NOs 9, 10 and 6 are MAB2, MAB3, MAB6 and MAB7.

In one embodiment, the antibody or antigen-binding fragment thereof of the present invention comprises:
a VH comprising the three following CDRs:
   CDR1: NHAMA (SEQ ID NO: 7);
   CDR2: YISYDGDNTYYADSVKG (SEQ ID NO: 11); and
   CDR3: GGNSGYD (SEQ ID NO: 3); and
a VL comprising the three following CDRs:
   CDR1: RASQNVNKFLN (SEQ ID NO: 9);
   CDR2: GTNSLQS (SEQ ID NO: 10); and
   CDR3: QQYTSWPWT (SEQ ID NO: 6).

In one embodiment, any of CDR1, CDR2 and/or CDR3 of the VH with SEQ ID NOs 7, 11, 3 and/or of the VL with SEQ ID NOs 6, 9-10 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

Examples of antibodies comprising a heavy chain comprising CDR1, CDR2 and CDR3 with SEQ ID NOs 7, 11 and 3 and a light chain comprising CDR1, CDR2 and CDR3 with SEQ ID NOs 9, 10 and 6 are MAB10 and MAB11.

In one embodiment, the antibody or antigen-binding fragment thereof of the present invention comprises:
a VH comprising the three following CDRs:
   CDR1: NHAMA (SEQ ID NO: 7)
   CDR2: YISYDGDNTYYADSVKG (SEQ ID NO: 11); and
   CDR3: GGNSGYD (SEQ ID NO: 3); and
and a VL comprising the three following CDRs:
   CDR1: KGSQNVNKFLN (SEQ ID NO: 12);
   CDR2: GTNSLQT (SEQ ID NO: 13); and
   CDR3: QQYTSWPWT (SEQ ID NO: 6).

In one embodiment, any of CDR1, CDR2 and/or CDR3 of the VH with SEQ ID NOs 7, 11, 3 and/or of the VL with SEQ ID NOs 6, 12-13 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

One example of an antibody comprising a heavy chain comprising CDR1, CDR2 and CDR3 with SEQ ID NOs 7, 11 and 3 and a light chain comprising CDR1, CDR2 and CDR3 with SEQ ID NOs 12, 13 and 6 is MAB9.

In one embodiment, the antibody or antigen-binding fragment thereof of the present invention comprises:
a VH comprising the three following CDRs:
   CDR1: SHAMA (SEQ ID NO: 14);
   CDR2: YISYDGDNTYYADSVKG (SEQ ID NO: 11); and
   CDR3: GGNSGYD (SEQ ID NO: 3); and
a VL comprising the three following CDRs:
   CDR1: KGSQNVNKFLN (SEQ ID NO: 12);
   CDR2: GTNSLQT (SEQ ID NO: 13); and
   CDR3: QQYTSWPWT (SEQ ID NO: 6).

In one embodiment, any of CDR1, CDR2 and/or CDR3 of the VH with SEQ ID NOs 14, 11, 3 and/or of the VL with SEQ ID NOs 6, 12-13 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

Examples of antibodies comprising a heavy chain comprising CDR1, CDR2 and CDR3 with SEQ ID NOs 14, 11 and 3 and a light chain comprising CDR1, CDR2 and CDR3 with SEQ ID NOs 12, 13 and 6 are MAB13 and MAB17.

In one embodiment, the antibody or antigen-binding fragment thereof of the present invention comprises:
a VH comprising the three following CDRs:
   CDR1: SHAMA (SEQ ID NO: 14);
   CDR2: YISYDGDNTYYADSVKG (SEQ ID NO: 11); and
   CDR3: GGNSGYD (SEQ ID NO: 3), and
a VL comprising the three following CDRs:
   CDR1: RASQNVNKFLN (SEQ ID NO: 9);
   CDR2: GTNSLQS (SEQ ID NO: 10); and
   CDR3: QQYTSWPWT (SEQ ID NO: 6).

In one embodiment, any of CDR1, CDR2 and/or CDR3 of the VH with SEQ ID NOs 14, 11, 3 and/or of the VL with SEQ ID NOs 6, 9-10 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

Examples of antibodies comprising a heavy chain comprising CDR1, CDR2 and CDR3 with SEQ ID NOs 14, 11 and 3 and a light chain comprising CDR1, CDR2 and CDR3 with SEQ ID NOs 9, 10 and 6 are MAB14, MAB15, MAB18 and MAB19.

In one embodiment, the antibody or antigen-binding fragment thereof according to the present invention comprises a VH comprising or consisting of the sequence SEQ ID NO: 17, wherein X₁ is N or S, X₂ is K or G, X₃ is R or A, X₄ is A or S, X₅ is Q or K and X₆ is S or N.

SEQ ID NO: 17
QVQLVESGGGVVQPGRSLRLSCAASGFTFSX₁HAMAWVRQAPX₂KGLEWV
AYISYDGDNTYYX₃DSVKGRFTISRDNX₄X₅X₆TLYLQMNSLRAEDTAVY
YCTTGGNSGYDWGQGTLVTVSS

In one embodiment, in SEQ ID NO: 17, X₁ is N or S, X₂ is G, X₃ is A, X₄ is A or S, X₅ is K and X₆ is S or N.

In one embodiment, the antibody or antigen binding fragment thereof according to the present invention comprises a VH comprising or consisting of the sequence SEQ ID NO: 41, wherein X₁ is K or G and X₂ is Q or K.

SEQ ID NO: 41
QVQLVESGGGVVQPGRSLRLSCAASGFTFSNHAMAWVRQAPX₁KGLEWV
AYISYDGDNTYYRDSVKGRFTISRDNAX₂STLYLQMNSLRAEDTAVYYC
TTGGNSGYDWGQGTLVTVSS

In one embodiment, the antibody or antigen binding fragment thereof according to the present invention comprises a VH comprising or consisting of the sequence SEQ ID NO: 43, wherein X₁ is S or N.

SEQ ID NO: 43
QVQLVESGGGVVQPGRSLRLSCAASGFTFSSHAMAWVRQAPGKGLEWVA
YISYDGDNTYYADSVKGRFTISRDNSKX₁TLYLQMNSLRAEDTAVYYCT
TGGNSGYDWGQGTLVTVSS

In one embodiment, the VH comprises or consists of the sequence SEQ ID NO: 17 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid. In one embodiment, the VH comprises or consists of the sequence SEQ ID NO: 41 or SEQ ID NO: 43 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid.

In one embodiment, the VH has an amino acid sequence that shares at least 70%, 75%, 80%, 85%, 90%, 95%, 96%, 97%, 98%, 99% or more of identity with SEQ ID NO: 17. In one embodiment, the VH has an amino acid sequence that shares at least 70%, 75%, 80%, 85%, 90%, 95%, 96%, 97%, 98%, 99% or more of identity with SEQ ID NO: 41 or SEQ ID NO: 43.

In one embodiment, the antibody or antigen-binding fragment thereof according to the present invention comprises a VL comprising or consisting of the sequence SEQ ID NO: 18, wherein X₁ is K or R, X₂ is G or A, X₃ is L or P, X₄ is E or K, X₅ is T or S and X₆ is Y or F.

SEQ ID NO: 18
DIQMTQSPSSLSASVGDRVTITCX₁X₂SQNVNKFLNWYQQKX₃GX₄APRR
LIYGTNSLQX₅GVPSRFSGSGSGTDX₆TLTISSLQPEDFATYYCQQYTSW
PWTFGQGTKLEIK

In one embodiment, in SEQ ID NO: 18, X₁ is R, X₂ is A, X₃ is P, X₄ is K, X₅ is S and X₆ is Y or F.

In one embodiment, the antibody or antigen-binding fragment thereof according to the present invention comprises a VL comprising or consisting of the sequence SEQ ID NO: 42, wherein X₁ is Y or F.

SEQ ID NO: 42
DIQMTQSPSSLSASVGDRVTITCRASQNVNKFLNWYQQKPGKAPRRLIY
GTNSLQSGVPSRFSGSGSGTDX₁TLTISSLQPEDFATYYCQQYTSWPWT
FGQGTKLEIK

In one embodiment, the VL comprises or consists of the sequence SEQ ID NO: 18 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids substituted by a different amino acid. In one embodiment, the VL comprises or consists of the sequence SEQ ID NO: 42 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids substituted by a different amino acid.

In one embodiment, the VL has an amino acid sequence that shares at least 70%, 75%, 80%, 85%, 90%, 95%, 96%, 97%, 98%, 99% or more of identity with SEQ ID NO: 18. In one embodiment, the VL has an amino acid sequence that shares at least 70%, 75%, 80%, 85%, 90%, 95%, 96%, 97%, 98%, 99% or more of identity with SEQ ID NO: 42.

In one embodiment, the antibody or antigen-binding fragment thereof according to the present invention comprises:
a VH comprising or consisting of the sequence SEQ ID NO: 17, wherein X₁ is N or S, X₂ is K or G, X₃ is R or A, X₄ is A or S, X₅ is Q or K and X₆ is S or N; and
a VL comprising or consisting of the sequence SEQ ID NO: 18, wherein X₁ is K or R, X₂ is G or A, X₃ is L or P, X₄ is E or K, X₅ is T or S and X₆ is Y or F, with the proviso that
when X₁ is N and X₃ is R in SEQ ID NO: 17, X₁ is R and/or X₅ is S in SEQ ID NO: 18, and
when X₁ is K and X₅ is T in SEQ ID NO: 18, X₁ is S and/or X₃ is A in SEQ ID NO: 17.

In one embodiment, the antibody or antigen-binding fragment thereof according to the present invention comprises:
a VH comprising or consisting of the sequence SEQ ID NO: 17, wherein X₁ is N or S, X₂ is G, X₃ is A, X₄ is A or S, X₅ is K and X₆ is S or N; and
a VL comprising or consisting of the sequence SEQ ID NO: 18, wherein X₁ is R, X₂ is A, X₃ is P, X₄ is K, X₅ is S and X₆ is Y or F.

In one embodiment, the antibody or antigen-binding fragment thereof according to the present invention comprises:
a VH comprising or consisting of the sequence SEQ ID NO: 41, wherein X₁ is K or G and X₂ is Q or K; and
a VL comprising or consisting of the sequence SEQ ID NO: 42, wherein X₁ is Y or F.

In one embodiment, the antibody or antigen-binding fragment thereof according to the present invention comprises:
a VH comprising or consisting of the sequence SEQ ID NO: 21; and
a VL comprising or consisting of the sequence SEQ ID NO: 42, wherein X₁ is Y or F.

In one embodiment, the antibody or antigen-binding fragment thereof according to the present invention comprises:
a VH comprising or consisting of the sequence SEQ ID NO: 43, wherein X₁ is S or N; and
a VL comprising or consisting of the sequence SEQ ID NO: 24.

In one embodiment, the antibody or antigen-binding fragment thereof according to the present invention comprises:
a VH comprising or consisting of the sequence SEQ ID NO: 43, wherein X₁ is S or N; and
a VL comprising or consisting of the sequence SEQ ID NO: 42, wherein X₁ is Y or F.

In one embodiment, the VH comprises or consists of the sequence SEQ ID NO: 17 and/or the VL comprises or consists of the sequence SEQ ID NO: 18 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid.

In one embodiment, the VH comprises or consists of the sequence SEQ ID NO: 41 and/or the VL comprises or consists of the sequence SEQ ID NO: 42 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid.

In one embodiment, the VH comprises or consists of the sequence SEQ ID NO: 21 and/or the VL comprises or consists of the sequence SEQ ID NO: 42 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid.

In one embodiment, the VH comprises or consists of the sequence SEQ ID NO: 43 and/or the VL comprises or consists of the sequence SEQ ID NO: 24 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid.

In one embodiment, the VH comprises or consists of the sequence SEQ ID NO: 43 and/or the VL comprises or consists of the sequence SEQ ID NO: 42 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid.

In one embodiment, the VH and/or the VL has/have an amino acid sequence that shares at least 70%, 75%, 80%, 85%, 90%, 95%, 96%, 97%, 98%, 99% or more of identity with SEQ ID NO: 17 and/or SEQ ID NO: 18, respectively.

In one embodiment, the VH and/or the VL has/have an amino acid sequence that shares at least 70%, 75%, 80%, 85%, 90%, 95%, 96%, 97%, 98%, 99% or more of identity with SEQ ID NO: 41 and/or SEQ ID NO: 42, respectively.

In one embodiment, the VH and/or the VL has/have an amino acid sequence that shares at least 70%, 75%, 80%, 85%, 90%, 95%, 96%, 97%, 98%, 99% or more of identity with SEQ ID NO: 21 and/or SEQ ID NO: 42, respectively.

In one embodiment, the VH and/or the VL has/have an amino acid sequence that shares at least 70%, 75%, 80%, 85%, 90%, 95%, 96%, 97%, 98%, 99% or more of identity with SEQ ID NO: 43 and/or SEQ ID NO: 24, respectively.

In one embodiment, the VH and/or the VL has/have an amino acid sequence that shares at least 70%, 75%, 80%, 85%, 90%, 95%, 96%, 97%, 98%, 99% or more of identity with SEQ ID NO: 43 and/or SEQ ID NO: 42, respectively.

As used herein, the phrase “characterized as having [ . . . ] amino acids being substituted by a different amino acid” in reference to a given sequence, refers to the occurrence, in said sequence, of conservative amino acid modifications.

As used herein, the expression “conservative amino acid modifications” refers to modifications that do not significantly affect or alter the binding characteristics of the antibody or antigen-binding fragment thereof containing the amino acid sequence. Such conservative modifications include amino acid substitutions, additions and deletions. Modifications can be introduced into an antibody or antigen-binding fragment thereof by standard techniques known in the art, such as site-directed mutagenesis and PCR-mediated mutagenesis.

Conservative amino acid substitutions are typically those in which an amino acid residue is replaced with an amino acid residue having a side chain with similar physicochemical properties. Specified variable region and CDR sequences may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acid insertions, deletions and/or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine) and aromatic side chains (e.g., tyrosine, phenylalanine, tryptophan, histidine). Thus, one or more amino acid residues within the CDRs and/or variable regions of the antibody or antigen-binding fragment thereof according to the present invention can be replaced with other amino acid residues from the same side chain family and the altered antibody can be tested for retained function (i.e., the properties set forth herein, such as, e.g., the binding to hCD25) using the assays described herein. In another embodiments, a string of amino acids within the CDRs and/or variable regions of the antibody or antigen-binding fragment thereof according to the present invention can be replaced with a structurally similar string that differs in order and/or composition of side chain family members.

In one embodiment, the antibody or antigen-binding fragment thereof according to the present invention comprises a VH comprising or consisting of a sequence selected from the group comprising SEQ ID NO: 19, SEQ ID NO: 20, SEQ ID NO: 21, SEQ ID NO: 22 and SEQ ID NO: 23.

SEQ ID NO: 19
QVQLVESGGGVVQPGRSLRLSCAASGFTFSNHAMAWVRQAPKKGLEWVA
YISYDGDNTYYRDSVKGRFTISRDNAQSTLYLQMNSLRAEDTAVYYCTT
GGNSGYDWGQGTLVTVSS
SEQ ID NO: 20
QVQLVESGGGVVQPGRSLRLSCAASGFTFSNHAMAWVRQAPGKGLEWVA
YISYDGDNTYYRDSVKGRFTISRDNAKSTLYLQMNSLRAEDTAVYYCTT
GGNSGYDWGQGTLVTVSS
SEQ ID NO: 21
QVQLVESGGGVVQPGRSLRLSCAASGFTFSNHAMAWVRQAPGKGLEWVA
YISYDGDNTYYADSVKGRFTISRDNAKSTLYLQMNSLRAEDTAVYYCTT
GGNSGYDWGQGTLVTVSS
SEQ ID NO: 22
QVQLVESGGGVVQPGRSLRLSCAASGFTFSSHAMAWVRQAPGKGLEWVA
YISYDGDNTYYADSVKGRFTISRDNSKSTLYLQMNSLRAEDTAVYYCTT
GGNSGYDWGQGTLVTVSS
SEQ ID NO: 23
QVQLVESGGGVVQPGRSLRLSCAASGFTFSSHAMAWVRQAPGKGLEWVA
YISYDGDNTYYADSVKGRFTISRDNSKNTLYLQMNSLRAEDTAVYYCTT
GGNSGYDWGQGTLVTVSS

In one embodiment, the VH comprises or consists of a sequence SEQ ID NO: 19, SEQ ID NO: 20, SEQ ID NO: 21, SEQ ID NO: 22 or SEQ ID NO: 23 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids substituted by a different amino acid.

In one embodiment, the VH has an amino acid sequence that shares at least 70%, 75%, 80%, 85%, 90%, 95%, 96%, 97%, 98%, 99% or more of identity with SEQ ID NO: 19, SEQ ID NO: 20, SEQ ID NO: 21, SEQ ID NO: 22 or SEQ ID NO: 23.

In one embodiment, the antibody or antigen-binding fragment thereof according to the present invention comprises a VL comprising or consisting of a sequence selected from the group comprising SEQ ID NO: 24, SEQ ID NO: 25 and SEQ ID NO: 26.

SEQ ID NO: 24
DIQMTQSPSSLSASVGDRVTITCKGSQNVNKFLNWYQQKLGEAPRRLIY
GTNSLQTGVPSRFSGSGSGTDYTLTISSLQPEDFATYYCQQYTSWPWTF
GQGTKLEIK
SEQ ID NO: 25
DIQMTQSPSSLSASVGDRVTITCRASQNVNKFLNWYQQKPGKAPRRLIY
GTNSLQSGVPSRFSGSGSGTDYTLTISSLQPEDFATYYCQQYTSWPWTF
GQGTKLEIK
SEQ ID NO: 26
DIQMTQSPSSLSASVGDRVTITCRASQNVNKFLNWYQQKPGKAPRRLIY
GTNSLQSGVPSRFSGSGSGTDFTLTISSLQPEDFATYYCQQYTSWPWTF
GQGTKLEIK

In one embodiment, the VL comprises or consists of a sequence SEQ ID NO: 24, SEQ ID NO: 25 or SEQ ID NO: 26 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids substituted by a different amino acid.

In one embodiment, the VL has an amino acid sequence that shares at least 70%, 75%, 80%, 85%, 90%, 95%, 96%, 97%, 98%, 99% or more of identity with SEQ ID NO: 24, SEQ ID NO: 25 or SEQ ID NO: 26.

In one embodiment, the antibody or antigen-binding fragment thereof according to the present invention comprises:
a VH comprising or consisting of a sequence selected from the group comprising SEQ ID NO: 19, SEQ ID NO: 20, SEQ ID NO: 21, SEQ ID NO: 22, SEQ ID NO: 23 and sequences with at least 70%, 75%, 80%, 85%, 90%, 95%, 96%, 97%, 98%, 99% or more of identity with SEQ ID NO: 19-23; and
a VL comprising or consisting of a sequence selected from the group comprising SEQ ID NO: 24, SEQ ID NO: 25, SEQ ID NO: 26 and sequences with at least 70%, 75%, 80%, 85%, 90%, 95%, 96%, 97%, 98%, 99% or more of identity with SEQ ID NO: 24-26,
with the proviso that
when VH comprises or consists of the sequence SEQ ID NO: 19 or SEQ ID NO: 20,
VL does not comprise or consist of the sequence SEQ ID NO: 24.

In one embodiment, the antibody or antigen-binding fragment thereof comprises:
a VH comprising or consisting of the sequence SEQ ID NO: 19; and
a VL comprising or consisting of the sequence SEQ ID NO: 25.

An example of such an antibody is MAB02.

In one embodiment, the antibody or antigen-binding fragment thereof comprises:
a VH comprising or consisting of the sequence SEQ ID NO: 19; and
a VL comprising or consisting of the sequence SEQ ID NO: 26.

An example of such an antibody is MAB03.

In one embodiment, the antibody or antigen-binding fragment thereof comprises:
a VH comprising or consisting of the sequence SEQ ID NO: 20; and
a VL comprising or consisting of the sequence SEQ ID NO: 25.

An example of such an antibody is MAB06.

In one embodiment, the antibody or antigen-binding fragment thereof comprises:
a VH comprising or consisting of the sequence SEQ ID NO: 20; and
a VL comprising or consisting of the sequence SEQ ID NO: 26.

An example of such an antibody is MAB07.

In one embodiment, the antibody or antigen-binding fragment thereof comprises:
a VH comprising or consisting of the sequence SEQ ID NO: 21; and
a VL comprising or consisting of the sequence SEQ ID NO: 24.

An example of such an antibody is MAB09.

In one embodiment, the antibody or antigen-binding fragment thereof comprises:
a VH comprising or consisting of the sequence SEQ ID NO: 21; and
a VL comprising or consisting of the sequence SEQ ID NO: 25.

An example of such an antibody is MAB10.

In one embodiment, the antibody or antigen-binding fragment thereof comprises:
a VH comprising or consisting of the sequence SEQ ID NO: 21; and
a VL comprising or consisting of the sequence SEQ ID NO: 26.

An example of such an antibody is MAB11.

In one embodiment, the antibody or antigen-binding fragment thereof comprises:
a VH comprising or consisting of the sequence SEQ ID NO: 22; and
a VL comprising or consisting of the sequence SEQ ID NO: 24.

An example of such an antibody is MAB13.

In one embodiment, the antibody or antigen-binding fragment thereof comprises:
a VH comprising or consisting of the sequence SEQ ID NO: 22; and
a VL comprising or consisting of the sequence SEQ ID NO: 25.

An example of such an antibody is MAB14.

In one embodiment, the antibody or antigen-binding fragment thereof comprises:
a VH comprising or consisting of the sequence SEQ ID NO: 22; and
a VL comprising or consisting of the sequence SEQ ID NO: 26.

An example of such an antibody is MAB15.

In one embodiment, the antibody or antigen-binding fragment thereof comprises:
a VH comprising or consisting of the sequence SEQ ID NO: 23; and
a VL comprising or consisting of the sequence SEQ ID NO: 24.

An example of such an antibody is MAB17.

In one embodiment, the antibody or antigen-binding fragment thereof comprises:
a VH comprising or consisting of the sequence SEQ ID NO: 23; and
a VL comprising or consisting of the sequence SEQ ID NO: 25.

An example of such an antibody is MAB18.

In one embodiment, the antibody or antigen-binding fragment thereof comprises:
a VH comprising or consisting of the sequence SEQ ID NO: 23; and
a VL comprising or consisting of the sequence SEQ ID NO: 26.

An example of such an antibody is MAB19.

In one embodiment, the VL and/or the VH further comprises a leader sequence, preferably located N terminally from the VL amino acid sequence or N terminally from the VH amino acid sequence. Examples of leader sequences include, but are not limited to, SEQ ID NO: 27 and 28.

SEQ ID NO: 27
MDIRLSLAFLVLFIKGVQC
SEQ ID NO: 28
MAAVQLLGLLLLWLPAMRC

In one embodiment, the VH comprises an amino acid leader sequence SEQ ID NO: 27 located N terminally from the VH amino acid sequence (such as, for example, SEQ ID NO: 17, SEQ ID NO: 19, SEQ ID NO: 20, SEQ ID NO: 21, SEQ ID NO: 22 or SEQ ID NO: 23). Other examples of VH amino acid sequences that may comprise the amino acid leader sequence SEQ ID NO: 27 include SEQ ID NO: 41 or SEQ ID NO: 43.

In one embodiment, the VL comprises an amino acid leader sequence SEQ ID NO: 28 located N terminally from the VL amino acid sequence (such as, for example, SEQ ID NO: 18, SEQ ID NO: 24, SEQ ID NO: 25 or SEQ ID NO: 26). Another example of a VL amino acid sequence that may comprise the amino acid leader sequence SEQ ID NO: 28 includes SEQ ID NO: 42.

The present invention further relates to a MAB02-like antibody, i.e., to an antibody binding the same epitope as MAB02, or substantially the same epitope than MAB02. The present invention thus further relates to an antibody competing with MAB02 for binding to CD25.

The present invention further relates to a MAB03-like antibody, i.e., to an antibody binding the same epitope as MAB03, or substantially the same epitope than MAB03. The present invention thus further relates to an antibody competing with MAB03 for binding to CD25.

The present invention further relates to a MAB06-like antibody, i.e., to an antibody binding the same epitope as MAB06, or substantially the same epitope than MAB06. The present invention thus further relates to an antibody competing with MAB06 for binding to CD25.

The present invention further relates to a MAB07-like antibody, i.e., to an antibody binding the same epitope as MAB07, or substantially the same epitope than MAB07. The present invention thus further relates to an antibody competing with MAB07 for binding to CD25.

The present invention further relates to a MAB09-like antibody, i.e., to an antibody binding the same epitope as MAB09, or substantially the same epitope than MAB09. The present invention thus further relates to an antibody competing with MAB09 for binding to CD25.

The present invention further relates to a MAB10-like antibody, i.e., to an antibody binding the same epitope as MAB10, or substantially the same epitope than MAB10. The present invention thus further relates to an antibody competing with MAB10 for binding to CD25.

The present invention further relates to a MAB11-like antibody, i.e., to an antibody binding the same epitope as MAB11, or substantially the same epitope than MAB11. The present invention thus further relates to an antibody competing with MAB11 for binding to CD25.

The present invention further relates to a MAB13-like antibody, i.e., to an antibody binding the same epitope as MAB13, or substantially the same epitope than MAB13. The present invention thus further relates to an antibody competing with MAB13 for binding to CD25.

The present invention further relates to a MAB14-like antibody, i.e., to an antibody binding the same epitope as MAB14, or substantially the same epitope than MAB14. The present invention thus further relates to an antibody competing with MAB14 for binding to CD25.

The present invention further relates to a MAB15-like antibody, i.e., to an antibody binding the same epitope as MAB15, or substantially the same epitope than MAB15. The present invention thus further relates to an antibody competing with MAB15 for binding to CD25.

The present invention further relates to a MAB17-like antibody, i.e., to an antibody binding the same epitope as MAB17, or substantially the same epitope than MAB17. The present invention thus further relates to an antibody competing with MAB17 for binding to CD25.

The present invention further relates to a MAB18-like antibody, i.e., to an antibody binding the same epitope as MAB18, or substantially the same epitope than MAB18. The present invention thus further relates to an antibody competing with MAB18 for binding to CD25.

The present invention further relates to a MAB19-like antibody, i.e., to an antibody binding the same epitope as MAB19, or substantially the same epitope than MAB19. The present invention thus further relates to an antibody competing with MAB19 for binding to CD25.

In one embodiment, the antibody or antigen-binding fragment thereof according to the present invention comprises a fully or substantially fully human heavy chain constant region (abbreviated herein as C_{H}) and/or light chain constant region (abbreviated herein as C_{L}). In one embodiment, the constant region is of human origin.

In one embodiment, the antibody or antigen-binding fragment thereof according to the present invention comprises a fully or substantially fully murine C_{H} and/or C_{L}. In one embodiment, the constant region is of murine origin.

In one embodiment, the antibody or antigen-binding fragment thereof according to the present invention is a murine antibody or fragment thereof.

In one embodiment, the antibody or antigen-binding fragment thereof according to the present invention is a chimeric antibody or fragment thereof.

A “chimeric antibody”, as used herein, refers to an antibody or antigen-binding fragment thereof comprising a first amino acid sequence linked to a second amino acid sequence with which it is not naturally linked in nature. The amino acid sequences may normally exist in separate proteins that are brought together in the fusion protein or may normally exist in the same protein but are placed in a new arrangement in the fusion protein. A chimeric protein may be created, for example, by chemical synthesis, or by creating and translating a polynucleotide in which the peptide regions are encoded in the desired relationship. The term “chimeric antibody” encompasses herein antibodies and antigen-binding fragment thereof in which
(a) the constant region, or a portion thereof, is altered, replaced or exchanged so that the variable region is linked to a constant region of a different or altered class, effector function and/or species, or an entirely different molecule which confers new properties to the chimeric antibody, e.g., an enzyme, toxin, hormone, growth factor, drug, etc.; or
(b) the variable region, or a portion thereof, is altered, replaced or exchanged with a variable region, or portion thereof, having a different or altered antigen specificity; or with corresponding sequences from another species or from another antibody class or subclass.

In one embodiment, the antibody or antigen-binding fragment thereof according to the present invention is a humanized antibody or fragment thereof.

A “humanized antibody”, as used herein, refers to a chimeric antibody or antigen-binding fragment thereof which contains minimal sequence derived from a non-human immunoglobulin. It includes antibodies made by a non-human cell having variable and constant regions which have been altered to more closely resemble antibodies that would be made by a human cell, e.g., by altering the non-human antibody amino acid sequence to incorporate amino acids found in human germline immunoglobulin sequences. Humanized antibodies or antigen-binding fragment thereof according to the present invention may include amino acid residues not encoded by human germline immunoglobulin sequences (e.g., mutations introduced by random or site-specific mutagenesis in vitro or by somatic mutation in vivo), for example in the CDRs. The term “humanized antibody” also includes antibodies and antigen-binding fragment thereof in which CDR sequences derived from the germline of another mammalian species, such as a mouse, have been grafted onto human framework sequences. In other words, the term “humanized antibody” refers to an antibody or antigen-binding fragment thereof in which the CDRs of a recipient human antibody are replaced by CDRs from a donor non-human antibody. Humanized antibodies or antigen-binding fragments thereof may also comprise residues of donor origin in the framework sequences. The humanized antibody or antigen-binding fragment thereof can also comprise at least a portion of a human immunoglobulin constant region. Humanized antibodies or antigen-binding fragments thereof may also comprise residues which are found neither in the recipient antibody nor in the imported CDR or framework sequences. Humanization can be performed using methods known in the art (e.g., Jones et al., 1986. Nature. 321(6069):522-5; Riechmann et al., 1988. Nature. 332(6162):323-7; Verhoeyen et al., 1988. Science. 239(4847):1534-6; Presta, 1992. Curr Opin Biotechnol. 3(4):394-8; patent U.S. Pat. No. 4,816,567), including techniques such as “superhumanizing” antibodies (e.g., Tan et al., 2002. J Immunol. 169(2):1119-25) and “resurfacing” (e.g., Staelens et al., 2006. Mol Immunol. 43(8):1243-57; Roguska et al., 1994. Proc Natl Acad Sci USA. 91(3):969-73).

A “humanized antibody” may retain a similar antigenic specificity as the original antibody. However, using certain methods of humanization, the affinity and/or specificity of binding of the antibody may be increased.

Methods for humanizing the antibody or antigen-binding fragment thereof according to the present invention are well-known in the art. The choice of human variable domains, both light and heavy, to be used in making the humanized antibody or antigen-binding fragment thereof is very important to reduce antigenicity. According to the so-called “best-fit” method, the sequence of the variable domain of an antibody or antigen-binding fragment thereof according to the present invention is screened against the entire library of known human variable-domain sequences. The human sequence that is closest to the mouse sequence is then accepted as the human framework (FR) for the humanized antibody (Sims et al., 1993. J Immunol. 151(4):2296-308; Chothia & Lesk, 1987. J Mol Biol. 196(4):901-17).

Another method for humanizing the antibody or antigen-binding fragment thereof according to the present invention uses a particular framework from the consensus sequence of all human antibodies of a particular subgroup of light or heavy chains. The same framework can be used for several different humanized antibodies (Carter et al., 1992. Proc Natl Acad Sci USA. 89(10):4285-9; Presta et al., 1993. J Immunol. 151(5):2623-32). It is further important that antibodies be humanized with retention of high affinity for hCD25 and other favorable biological properties. To achieve this goal, according to a preferred method, humanized antibodies and antigen-binding fragments thereof are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its epitope. In this way, CDR residues can be selected and combined from the consensus and import sequences so that the desired antibody characteristic, such as an increased affinity for hCD25, is achieved. In general, the CDR residues are directly and most substantially involved in influencing antigen binding.

Another method for humanizing the antibody or antigen-binding fragment thereof according to the present invention is to use a transgenic or transchromosomic animal carrying parts of the human immune system for immunization. As a host, these animals have had their immunoglobulin genes replaced by functional human immunoglobulin genes. Thus, antibodies produced by these animals or in hybridomas made from the B cells of these animals are already humanized. Examples of such transgenic or transchromosomic animal include, without limitation:
the XenoMouse (Abgenix, Fremont, CA), described in U.S. Pat. Nos. 5,939,598, 6,075,181, 6,114,598, 6,150,584 and 6,162,963;
the HuMAb Mouse® (Medarex, Inc.), described in Lonberg et al., 1994. Nature. 368(6474):856-859; Lonberg & Huszar, 1995. Int Rev Immunol. 13(1):65-93; Harding & Lonberg, 1995. Ann N Y Acad Sci. 764:536-46; Taylor et al., 1992. Nucleic Acids Res. 20(23):6287-95; Chen et al., 1993. Int Immunol. 5(6):647-56; Tuaillon et al., 1993. Proc Natl Acad Sci USA. 90(8):3720-4; Choi et al., 1993. Nat Genet. 4(2):117-23; Chen et al., 1993. EMBO J. 12(3):821-30; Tuaillon et al., 1994. J Immunol. 152(6):2912-20; Taylor et al., 1994. Int Immunol. 6(4):579-91; Fishwild et al., 1996. Nat Biotechnol. 14(7):845-51;
the KM Mouse®, described in patent application WO2002043478;
the TC mice, described in Tomizuka et al., 2000. Proc Natl Acad Sci USA. 97(2):722-7; and
the OmniRat™ (OMT, Inc.), described in patent application WO2008151081; Geurts et al., 2009. Science. 325(5939):433; Menoret et al., 2010. Eur J Immunol. 40(10):2932-41.

Humanized antibodies and antigen-binding fragments thereof may also be produced according to various other techniques, such as by using, for immunization, other transgenic animals that have been engineered to express a human antibody repertoire (Jakobovitz et al., 1993. Nature. 362(6417):255-8), or by selection of antibody repertoires using phage display methods. Such techniques are known to the skilled person and can be implemented starting from monoclonal antibodies or antigen-binding fragments thereof as disclosed in the present application.

In some embodiments, the antibody or antigen-binding fragment thereof comprising VH and VL or CDRs thereof may comprise a first constant domain (C_{H}1 and/or C_{L}), the amino acid sequence of which is fully or substantially human.

In one embodiment, the antibody or antigen-binding fragment thereof according to the present invention is a fully or substantially human antibody or fragment thereof.

In some embodiments, especially when the antibody or antigen-binding fragment thereof according to the present invention is intended for human therapeutic uses, it is typical for the entire constant region, or at least a part thereof, to have a fully or substantially human amino acid sequence. Therefore, one or more of, or any combination of, the C_{H}1 domain, hinge region, C_{H}2 domain, C_{H}3 domain and C_{L} domain (and C_{H}4 domain if present) may be fully or substantially human with respect to its amino acid sequence. Advantageously, the C_{H}1 domain, hinge region, C_{H}2 domain, C_{H}3 domain and C_{L} domain (and C_{H}4 domain if present) may all have a fully or substantially human amino acid sequence.

The term “substantially human”, in the context of the constant region of a humanized or chimeric antibody or antigen-binding fragment thereof, refers to an amino acid sequence identity of at least 70%, preferably at least 75%, 80%, 85%, 90%, 95%, 96%, 97%, 98%, 99% or more with a human constant region.

The term “human amino acid sequence”, in this context, refers to an amino acid sequence which is encoded by a human immunoglobulin gene, which includes germline, rearranged and somatically mutated genes. The present invention also contemplates proteins comprising constant domains of “human” sequence which have been altered, by one or more amino acid additions, deletions or substitutions with respect to the human sequence, excepting those embodiments where the presence of a “fully human hinge region” is expressly required.

The presence of a “fully human hinge region” in the antibody or antigen-binding fragment thereof according to the present invention may be beneficial both to minimize immunogenicity and to optimize stability of the antibody. It is considered that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g., by addition or deletion of N- or O-linked glycosylation sites). Depending on the intended use of the antibody or antigen-binding fragment thereof, it may be desirable to modify the antibody or antigen-binding fragment thereof according to the present invention with respect to its binding properties to Fe receptors, for example to modulate effector function. For example, cysteine residue(s) may be introduced in the Fc region, thereby allowing interchain disulfide bond formation in this region. The homodimeric antibody thus generated may have improved effector function (Caron et al., 1992. J Exp Med. 176(4):1191-5; Shopes, 1992. J Immunol. 148(9):2918-22).

In one embodiment, the antibody or antigen-binding fragment thereof is from the IgG class.

In one embodiment, the antibody or antigen-binding fragment thereof is from the human IgG1 subclass. In one embodiment, the antibody or antigen-binding fragment thereof according to the present invention is thus an IgG1 antibody, preferably a human IgG1 antibody.

In another embodiment, the antibody or antigen-binding fragment thereof is from the human IgG2 subclass.

The Fc region of IgG antibodies interacts with cellular Fcγ receptors (FcγR) to stimulate and regulate downstream effector mechanisms. There are five activating receptors, namely FcγRI (CD64), FcγRIIa (CD32a), FcγRIIc (CD32c), FcγRIIIa (CD16a) and FcγRIIIb (CD16b), and one inhibitory receptor FcγRIIb (CD32b). The communication of IgG antibodies with the immune system is controlled and mediated by FcγRs, which relay the information sensed and gathered by antibodies to the immune system, providing a link between the innate and adaptive immune systems, and particularly in the context of biotherapeutics (Hayes J et al., 2016. J Inflamm Res 9: 209-219).

IgG subclasses vary in their ability to bind to FcγR and this differential binding determines their ability to elicit a range of functional responses. For example, in humans, FcγRIIIa is the major receptor involved in the activation of antibody-dependent cell-mediated cytotoxicity (ADCC) and IgG3 (followed closely by IgG1) display the highest affinities for this receptor, reflecting their ability to potently induce ADCC. IgG2 have been shown to have weak binding for this receptor.

In one embodiment, the antibody of the present invention or the antigen-binding fragment thereof binds FcγR with high affinity, preferably binds an activating receptor with high affinity.

In one embodiment, the antibody of the present invention or the antigen-binding fragment thereof binds FcγRI and/or FcγRIIa and/or FcγRIIc and/or FcγRIIIa and/or FcγRIIIb with high affinity.

In one embodiment, the antibody of the present invention or the antigen-binding fragment thereof is an IgG1 antibody (preferably a human IgG1 antibody) or a fragment thereof, and binds to at least one Fc activating receptor. For example, the antibody or the antigen-binding fragment thereof may bind to one or more receptor selected from FcγRI, FcγRIIa, FcγRIIc, FcγRIIIa and FcγRIIIb. In one embodiment, the antibody or the antigen-binding fragment thereof is capable of binding to FcγRIIIa. In one embodiment, the antibody or the antigen-binding fragment thereof is capable of binding to FcγRIIa. In one embodiment, the antibody or the antigen-binding fragment thereof is capable of binding to FcγRIIIa, FcγRIIc and optionally FcγRI. In one embodiment, the antibody or the antigen-binding fragment thereof is capable of binding to FcγRIIIa, FcγRIIa and optionally FcγRI.

In one embodiment, the antibody of the present invention or the antigen-binding fragment thereof binds to at least one activating Fcγ receptor with a dissociation constant of less than about 10⁻⁶ M, 10⁻⁷ M, 10⁻⁸ M, 10⁻⁹ M or 10⁻¹⁰ M.

In one embodiment, the antibody of the present invention or the antigen-binding fragment thereof is an IgG1 antibody (preferably a human IgG1 antibody) or a fragment thereof and binds to FcγRI, FcγRIIa, FcγRIIc, FcγRIIIa, and/or FcγRIIIb with a higher affinity than it binds to FcγRIIb, with low affinity.

In one embodiment, the antibody or antigen-binding fragment thereof according to the present invention may comprise human heavy chain constant regions sequences and allow to target, block, and/or deplete CD25-expressing cells to which they are bound.

In one embodiment, the proteins, antibodies or antigen-binding fragments thereof according to the present invention deplete CD25-expressing cells to which they are bound. In one embodiment, the proteins, antibodies or antigen-binding fragments thereof according to the present invention deplete Tregs to which they are bound. In one embodiment, the proteins, antibodies or antigen-binding fragments thereof according to the present invention also deplete or reduce tumor infiltrating regulatory T cells to which they are bound.

The term “deplete” or “depleting”, with respect to CD25-expressing cells or Tregs refers to the killing, elimination, lysis or induction of such killing, elimination or lysis, so as to negatively affect the number or proportion of CD25 expressing cells present in a sample or in a subject. In one embodiment, the protein, antibody or antigen binding fragment thereof according to the present invention allows targeting, blocking proliferation, and/or depleting CD25-expressing cells or Treg cells. In one embodiment, the depletion is via ADCC. In one embodiment, the depletion is via ADCP. In one embodiment, the depletion is via CDC.

Thus, in one embodiment, the protein, the antibody or the antigen-binding fragment of the antibody of the present invention leads, directly or indirectly, to the depletion of CD25-expressing cells (e.g., leads to a 10%, 20%, 30%, 40%, 45%, 50%, 55%, 60%, 65%, 70%, 80%, 85% or greater elimination or decrease in number of CD25 expressing cells).

In one embodiment, the protein, the antibody or the antigen-binding fragment of the antibody does not inhibit the binding of interleukin-2 (IL-2) to CD25 and depletes Tregs to which they are bound.

In one embodiment, the antibody or antigen-binding fragment thereof according to the present invention induces antibody dependent cellular cytotoxicity (ADCC).

The term “antibody-dependent cell-mediated cytotoxicity” or “ADCC” refers to a cell-mediated cytotoxicity induced in an antibody-dependent manner when the Fc region of said antibody bound to its antigen binds to the Fc receptor on effector cells such as natural killer cells, macrophages, neutrophils, eosinophils and mononuclear cells (e.g., peripheral blood mononuclear cells), thereby leading to lysis of the target cell. ADCC can be measured using assays that are known and available in the art (e.g., Clynes et al. (1998) Proc Natl Acad Sci USA 95, 652-6).

In one embodiment, the antibody or antigen-binding fragment thereof according to the present invention is from the IgG1 (preferably human IgG1) subclass and has ADCC activity.

In one embodiment, the antibody or antigen-binding fragment thereof according to the present invention induces antibody-dependent cell-mediated phagocytosis (ADCP).

The term “antibody-dependent cell-mediated phagocytosis” (ADCP) or “opsonisation” refers to a cell-mediated reaction in which nonspecific cytotoxic cells (e.g., phagocytes, macrophages) that express Fc receptors (FcRs) recognize antibody bound on a target cell and induce phagocytosis of the target cell. ADCP can be measured using assays that are known and available in the art (e.g., Clynes et al. (1998) Proc Natl Acad Sci USA 95, 652-6).

In one embodiment, the antibody or antigen-binding fragment thereof according to the present invention is from the IgG1 (preferably human IgG1) subclass and has ADCP activity.

In one embodiment, the antibody or antigen-binding fragment thereof according to the present invention induces complement-dependent cytotoxicity (CDC).

The term “complement-dependent cytotoxicity” (CDC) refers to the induction of the lysis of antigen-expressing cells recognized by an antibody or antigen-binding fragment thereof of the invention in the presence of complement. The complement activation pathway is initiated by the binding of the first component of the complement system (C1q) to a molecule (e.g., an antibody) complexed with a cognate antigen. CDC can be measured using assays that are known and available in the art (e.g., Clynes et al. (1998) Proc Natl Acad Sci USA 95, 652-6; Gazzano-Santaro et al., J. Immunol. Methods, 202:163 (1996)).

In one embodiment, the antibody or antigen-binding fragment thereof according to the present invention is from the IgG1 (preferably human IgG1) subclass and has CDC activity.

The constant region of an antibody is important in the ability of an antibody to fix complement and mediate cell-dependent cytotoxicity and phagocytosis. Thus, as discussed herein, the isotype of an antibody may be selected on the basis of whether it is desirable for the antibody to mediate cytotoxicity/phagocytosis.

In one embodiment, the antibody or antigen-binding fragment thereof according to the present invention is conjugated, such as, for example, to a toxic moiety. In one embodiment, the antibody or antigen-binding fragment thereof according to the present invention is linked to a toxic moiety.

In one embodiment, the antibody or antigen-binding fragment thereof according to the present invention is not conjugated, such as, for example, to a toxic moiety. In one embodiment, the antibody or antigen-binding fragment thereof according to the present invention is not linked to a toxic moiety.

In one embodiment, the antibody or antigen-binding fragment thereof according to the present invention lacks an Fc domain (e.g., lacks a C_{H}2 and/or C_{H}3 domain) or comprises an Fc domain of IgG2 or IgG4 isotype (preferably of human IgG2 or IgG4).

In one embodiment, the antibody or antigen-binding fragment thereof according to the present invention does not comprise an Fc region that mediates ADCC, ADCP and/or CDC.

In one embodiment, the antibody or antigen-binding fragment thereof according to the present invention does not induce ADCC, ADCP and/or CDC.

Thus, in one embodiment, the antibody or antigen-binding fragment thereof according to the present invention does not lead, directly or indirectly, to the depletion of CD25−expressing cells (e.g., do not lead to a 10%, 20%, 50%, 60% or greater elimination or decrease in number of CD25 cells). For example, the antibody of the present invention does not comprise an Fe domain capable of substantially binding to an FcγRIIIA (CD16) polypeptide.

In one embodiment, the antibody or antigen-binding fragment thereof according to the present invention is an engineered antibody or fragment thereof.

Engineered antibodies of the present invention include those in which modifications have been made to framework residues within VH and/or VL, e.g., to improve the properties of the antibody. Typically, such framework modifications are made to decrease the immunogenicity of the antibody. For example, one approach is to “back-mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis or PCR-mediated mutagenesis. Such “back-mutated” antibodies are also intended to be encompassed by the invention. Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In one embodiment, the antibody or antigen-binding fragment thereof according to the present invention is engineered to elicit an enhanced, increased or improved ADCC, ADCP, and/or CDC response.

As used herein, the term “enhanced, increased or improved ADCC, ADCP, and/or CDC response” is relative to the ADCC, ADCP, and/or CDC response induced by the antibody or fragment thereof according to the invention as compared the ADCC, ADCP, and/or CDC response induced with other anti-CD25 antibodies, including those that do not inhibit the binding of IL-2 to CD25 and, for example unmodified anti-CD25 monoclonal antibodies.

Methods to increase ADCC, ADCP and/or CDC are well known in the art. For example, ADCC may be increased by methods that eliminate the fucose moiety from the antibody glycan, such as by production of the antibody in a YB2/0 cell line, or though the introduction of specific mutations on the Fc portion of human IgG1 (e.g., S298A/E333A/K334A, S239D/I332E/A330L, G236A/S239D/A330L/I332E) (Lazar et al. (2006) Proc Natl Acad Sci USA 103, 2005-2010; Smith et al. (2012) Proc Natl 25 Acad Sci USA 109, 6181-6). ADCP may also be increased by the introduction of specific mutations on the Fc portion of human IgG1 (Richards et al. (2008) Mol Cancer Ther 7, 2517-27). CDC response may be increased with mutations in the antibody that increase the affinity of C1q binding (Idusogie et al. (2001) J Immunol 166, 2571-5).

Of note, methods to decrease or abolish ADCC, ADCP and/or CDC are also well known in the art. For example, ADCC may be decreased or abolished by methods modifying the glycosylation profile of the Fc domain of the immunoglobulin. CDC can be decreased or abolished by the replacement of one or more amino acids by other amino acid such that the antibody has altered C2q binding (U.S. Pat. No. 6,194,551 by Idusogie et al.).

In one embodiment, the antibody or antigen-binding fragment thereof according to the present invention is engineered to modify its glycosylation. For example, the antibody according to the invention is a glycosylted (i.e., the antibody lacks glycosylation). Glycosylation can be altered to, for example, increase the affinity of the antibody for the antigen or alter the ADCC activity of the antibody. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al (incorporated herein by reference). Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered fucosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP1176195 (incorporated herein by reference) describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the antibody or antigen-binding fragment thereof of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 (incorporated herein by reference) describes a variant CHO cell line, Lec13 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 (incorporated herein by reference) describes cell lines engineered to express glycoprotein-modifying glycosyl transferases (e.g., beta(1,4)—N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the antibody (preferably the monoclonal antibody) of the present invention can be produced in yeasts or filamentous fungi engineered for mammalian-like glycosylation pattern and capable of producing antibodies lacking fucose as glycosylation pattern (see for example EP1297172B1).

In one embodiment, the antibody or antigen-binding fragment thereof according to the present invention is a pegylated antibody or fragment thereof.

An antibody can be pegylated to, for example, increase the biological (e.g., serum) half-life of the antibody. To pegylate an antibody or a fragment thereof, the antibody or antibody fragment typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (DY12-DY120) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the present invention, such as, for example, as described in EP0154316 and EP0401384 (incorporated herein by reference).

The present invention further relates to a fusion protein comprising a protein as described herein, in particular an antibody or antigen-binding fragment thereof as described herein.

In one embodiment, said fusion protein comprises a second antigen binding moiety. In one embodiment, said fusion protein is a multispecific antibody, for example a bispecific antibody.

In one embodiment, the antibody or antigen-binding fragment thereof is bispecific, and is further capable of binding to another molecule.

In one embodiment, the other molecule is an immune receptor. Examples of immune receptors that may be bound by a bispecific antibody of the present invention include, but are not limited to CTLA4, PD-1, PD-L1, TIM-3, LAG-3, B7H3, B7H4, B7H6, 4-1BB, OX40, ICOS, GITR, TIGIT, CD27-CD70, CD40, BTLA, HVEM, CD160, CCR8 and CEACAM-1.

In one embodiment, the antibody or antigen-binding fragment thereof is bispecific, and is further capable of binding to a costimulatory molecule. Examples of costimulatory molecules include, but are not limited to, 4-1BB, ICOS, GITR, CD27-CD70, CD40 and OX40.

In one embodiment, the antibody or antigen-binding fragment thereof is bispecific, and is capable of binding to OX40. In one embodiment, the antibody or antigen-binding fragment thereof is bispecific, and is capable of binding to GITR. In one embodiment, the antibody or antigen-binding fragment thereof is bispecific, and is capable of binding to ICOS.

In another embodiment, the antibody or antigen-binding fragment thereof is bispecific, and is further capable of binding to a coinhibitory molecule. Examples of coinhibitory molecules include, but are not limited to, CTLA4, PD-1, PD-L1, TIM-3, LAG-3, TIGIT, BTLA, HVEM, CD160 and CEACAM-1.

In one embodiment, the antibody or antigen-binding fragment thereof is bispecific, and is capable of binding to CTLA4. In one embodiment, the antibody or antigen-binding fragment thereof is bispecific, and is capable of binding to PD-1. In one embodiment, the antibody or antigen-binding fragment thereof is bispecific, and is capable of binding to TIGIT.

In one embodiment, said fusion protein comprises a second antigen binding moiety that binds an immune checkpoint protein. Immune checkpoint proteins include checkpoint inhibitors and checkpoint agonists.

Checkpoint inhibitors (CPI, that may also be referred to as immune checkpoint inhibitors or ICI) molecules, often antibodies, block the interactions between inhibitory receptors (IRs) expressed on T cells and their ligands.

Examples of checkpoint inhibitors include, without being limited to, inhibitors of the cell surface receptor PD-1 (programmed cell death protein 1), also known as CD279 (cluster differentiation 279); inhibitors of the ligand PD-L1 (programmed death-ligand 1), also known as CD274 (cluster of differentiation 274) or B7-H1 (B7 homolog 1); inhibitors of the cell surface receptor CTLA4 or CTLA-4 (cytotoxic T-lymphocyte-associated protein 4), also known as CD152 (cluster of differentiation 152); inhibitors of LAG-3 (lymphocyte-activation gene 3), also known as CD223 (cluster differentiation 223); inhibitors of TIM-3 (T-cell immunoglobulin and mucin-domain containing-3), also known as HAVCR2 (hepatitis A virus cellular receptor 2) or CD366 (cluster differentiation 366); inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3); inhibitors of BTLA (B and T lymphocyte attenuator), also known as CD272 (cluster differentiation 272); inhibitors of CEACAM-1 (carcinoembryonic antigen-related cell adhesion molecule 1) also known as CD66a (cluster differentiation 66a).

Checkpoint agonists act by activating stimulatory receptors (costimulatory receptors) expressed on immune cells, such as T cells. As used herein, the term “stimulatory receptors” refers to receptors that induce a stimulatory signal upon activation, and thus lead to an enhancement of the immune response.

Examples of checkpoint agonists include, without being limited to, agonists of CD137 (cluster differentiation 137) also known as 4-1BB or TNFRS9 (tumor necrosis factor receptor superfamily, member 9); agonists of OX40 receptor also known as CD134 (cluster differentiation 134) or TNFRSF4 (tumor necrosis factor receptor superfamily, member 4); agonists of GITR (glucocorticoid-induced TNF receptor family-related protein); agonists of ICOS (inducible co-stimulator); agonists of CD27-CD70 (cluster differentiation 27-cluster differentiation 70); and agonists of CD40 (cluster differentiation 40).

In one embodiment, said fusion protein comprises a second antigen binding moiety that binds a T cell marker, such as, for example, CD2, CD3 or CD28.

In one embodiment, said fusion protein comprises a second antigen binding moiety that binds a NK cell marker, such as, for example, an activating NK receptor. Examples of activating NK receptors include, but are not limited to, activating forms of KIR proteins (for example KIR2DS proteins), CD160-TM, NKG2D, IL-2R, IL-12R, IL-15R, IL-18R and IL-21R.

In one embodiment, the antibody or antigen-binding fragment thereof is conjugated with a therapeutic moiety, i.e., a drug. The therapeutic moiety can be, e.g., a chemotherapeutic agent, an immunosuppressant, a lytic peptide, a radionuclide or a toxin. In one embodiment, the fusion protein thus comprises a therapeutic moiety and a protein, antibody or antigen-binding fragment thereof as described herein.

In one embodiment, the antibody or antigen-binding fragment thereof is not conjugated with a radionuclide (i.e., the antibody or antigen-binding fragment thereof is not radiolabeled) and/or with a toxin.

Examples of radionuclides include, but are not limited to, ⁹⁰Y ¹³¹I, or ⁶⁷Cu.

Examples of toxins include, but are not limited to, doxorubicin and calicheamicin.

In one embodiment, the antibody or antigen-binding fragment thereof is conjugated with a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065); an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In one embodiment, the antibody or antigen-binding fragment thereof is conjugated with a cytokine. Suitable cytokines include, but are not limited to, interferons, interleukins and colony-stimulating factors. In one embodiment, the fusion protein thus comprises a cytokine and a protein, antibody or antigen-binding fragment thereof as described herein.

In one embodiment, the antibody or antigen-binding fragment thereof is conjugated with a cytokine mimetic. In one embodiment, the fusion protein thus comprises a cytokine mimetic and a protein, antibody or antigen-binding fragment thereof as described herein.

Techniques for conjugating molecule to antibodies or antigen-binding fragments thereof are well-known in the art. Typically, the nucleic acid molecule is covalently attached to lysines or cysteines on the antibody or fragment thereof, through N-hydroxysuccinimide ester or maleimide functionality respectively. Methods of conjugation using engineered cysteines or incorporation of unnatural amino acids have been reported to improve the homogeneity of the conjugate.

Another object of the invention is an isolated nucleic acid encoding the isolated protein, in particular the antibody or antigen-binding fragment thereof binding to human CD25 according to the present invention.

Another object of the invention is an isolated nucleic acid encoding the fusion protein according to the present invention.

An “isolated nucleic acid”, as used herein, is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

In one embodiment, the isolated nucleic acid is purified.

In one embodiment, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A_{260/280})), and most preferably greater than 96%, 97%, 98% or 99% by weight; or
(2) homogeneity as shown by agarose gel electrophoresis and using an intercalating agent such as ethidium bromide, SYBR Green, GelGreen or the like.

In one embodiment, the nucleic acid encodes at least a heavy chain variable region or a light chain variable region of the antibody or antigen-binding fragment thereof according to the present invention. In one embodiment, the nucleic acid may encode variable and constant regions of the antibody or antigen-binding fragment thereof according to the present invention. In one embodiment, the nucleic acid may encode heavy and light chains of the antibody or antigen-binding fragment thereof on separate nucleic acids or on the same nucleic acid molecule.

In one embodiment, the nucleic acid according to the present invention comprises or consists of a sequence encoding the VH of the antibody or antigen-binding fragment thereof according to the invention.

In one embodiment, the nucleic acid according to the present invention comprises or consists of a sequence encoding the VH of the antibody or antigen-binding fragment thereof according to the invention, wherein said sequence is selected from the group comprising or consisting of SEQ ID NO: 29, SEQ ID NO: 30, SEQ ID NO: 31, SEQ ID NO: 32, SEQ ID NO: 33 and any sequence sharing at least 70%, 75%, 80%, 85%, 90%, 95%, 96%, 97%, 98%, 99% or more identity with SEQ ID NO: 29-33.

SEQ ID NO: 29
CAAGTGCAGCTGGTCGAATCCGGAGGCGGAGTGGTCCAGCCCGGCAGAT
CTCTGAGACTGAGCTGTGCTGCCTCCGGCTTCACATTCAGCAACCACGC
TATGGCTTGGGTGAGACAAGCCCCTAAGAAGGGACTGGAATGGGTGGCC
TACATCAGCTACGACGGCGACAACACATACTATAGGGACAGCGTCAAGG
GAAGGTTCACTATCTCTAGGGACAATGCCCAGAGCACTCTGTATCTGCA
GATGAATTCTCTGAGGGCCGAGGATACAGCCGTCTACTACTGCACAACT
GGCGGCAATAGCGGCTACGATTGGGGCCAAGGCACTCTGGTGACAGTGA
GCAGC
SEQ ID NO: 30
CAAGTGCAGCTGGTGGAAAGCGGAGGAGGCGTCGTGCAGCCCGGAAGGT
TCTGAGACTGAGCTGTGCCGCCAGCGGCTTCACATTCAGCAACCACGCC
ACTGGCTTGGGTGAGACAAGCCCCCGGCAAAGGACTGGAGTGGGTGGCC
TACATCTCCTACGACGGCGACAACACTTACTATAGGGACAGCGTCAAGG
GCAGATTCACAATCTCTAGGGACAACGCCAAGAGCACTCTGTATCTGCA
GATGAACTCTCTGAGGGCTGAGGATACAGCCGTGTACTACTGCACTACT
GGCGGCAACAGCGGCTATGATTGGGGCCAAGGCACTCTGGTCACTGTGA
GCAGC
SEQ ID NO: 31
CAAGTGCAGCTGGTGGAATCCGGAGGCGGCGTGGTGCAACCCGGCAGAT
CTCTGAGACTGAGCTGTGCCGCCAGCGGCTTCACTTTCAGCAATCACGC
CATGGCTTGGGTGAGACAAGCCCCCGGCAAGGGACTGGAGTGGGTCGCC
TACATCTCCTACGACGGCGACAACACATACTACGCCGACAGCGTCAAGG
GAAGGTTCACTATCTCTAGGGACAATGCCAAGAGCACACTGTATCTGCA
GATGAACTCTCTGAGGGCCGAGGACACTGCCGTGTACTACTGCACAACT
GGCGGCAATAGCGGCTACGATTGGGGCCAAGGCACTCTGGTGACAGTGA
GCAGC
SEQ ID NO: 32
CAAGTGCAGCTGGTGGAGAGCGGCGGAGGAGTGGTGCAACCCGGAAGGT
CTCTGAGACTGAGCTGTGCCGCCAGCGGCTTTACTTTCAGCAGCCACGC
TATGGCTTGGGTGAGGCAAGCCCCCGGCAAGGGACTGGAGTGGGTCGCC
TACATCTCCTACGACGGCGACAACACATACTACGCCGACAGCGTCAAGG
GAAGGTTCACAATCTCTAGGGACAACAGCAAGAGCACTCTGTATCTGCA
GATGAACTCTCTGAGAGCCGAGGACACAGCCGTCTACTACTGCACAACT
GGCGGCAATAGCGGCTACGATTGGGGCCAAGGCACTCTGGTGACAGTGA
GCAGC
SEQ ID NO: 33
CAAGTGCAGCTGGTGGAAAGCGGAGGCGGCGTGGTGCAACCCGGCAGAT
CTCTGAGACTGAGCTGTGCCGCCAGCGGCTTCACATTCAGCTCCCACGC
CATGGCTTGGGTGAGACAAGCCCCCGGCAAAGGACTGGAGTGGGTCGCC
TACATCTCCTACGACGGCGACAACACATACTACGCCGATAGCGTGAAGG
GAAGGTTCACTATCTCTAGGGACAACAGCAAGAACACTCTGTATCTGCA
GATGAACTCTCTGAGGGCTGAGGATACAGCCGTCTACTACTGCACAACT
GGCGGCAATAGCGGCTATGATTGGGGCCAAGGCACTCTGGTGACAGTGA
GCAGC

In one embodiment, the nucleic acid according to the present invention comprises or consists of a sequence encoding the VL of the antibody or antigen-binding fragment thereof according to the invention.

In one embodiment, the nucleic acid according to the present invention comprises or consists of a sequence encoding the VL of the antibody or antigen-binding fragment thereof according to the invention, wherein said sequence is selected from the group comprising or consisting of SEQ ID NO: 34, SEQ ID NO: 35, SEQ ID NO: 36 and any sequence sharing at least 70%, 75%, 80%, 85%, 90%, 95%, 96%, 97%, 98%, 99% or more identity with SEQ ID NO: 34-36.

SEQ ID NO: 34
GATATTCAGATGACACAGAGCCCTAGCTCTCTGAGCGCTAGCGTGGGAG
ATAGGGTGACAATCACATGCAAGGGCAGCCAGAACGTGAACAAGTTTCT
GAACTGGTATCAGCAGAAGCTGGGCGAGGCCCCTAGGAGGCTGATCTAC
GGCACAAACTCTCTGCAGACTGGCGTCCCAAGCAGATTTAGCGGCAGCG
GAAGCGGCACTGACTACACACTGACAATCAGCTCTCTGCAGCCAGAGGA
CTTCGCCACATACTACTGCCAGCAGTACACAAGCTGGCCTTGGACTTTC
GGCCAAGGCACTAAGCTGGAGATCAAG
SEQ ID NO: 35
GATATCCAGATGACTCAGAGCCCTTCCTCTCTGAGCGCTAGCGTGGGAG
ATAGGGTGACTATCACATGTAGGGCCAGCCAGAACGTCAACAAGTTTCT
GAACTGGTATCAGCAGAAGCCCGGCAAGGCCCCAAGGAGGCTGATCTAC
GGCACTAACTCTCTGCAGAGCGGCGTGCCAAGCAGATTTAGCGGCAGCG
GAAGCGGCACTGACTACACACTGACTATCAGCTCTCTGCAGCCAGAGGA
CTTCGCCACATACTACTGCCAGCAGTACACTAGCTGGCCTTGGACATTC
GGCCAAGGCACAAAGCTGGAGATCAAA
SEQ ID NO: 36
GACATCCAGATGACACAGAGCCCTAGCTCTCTGAGCGCTAGCGTGGGAG
ATAGGGTGACAATCACATGTAGGGCCAGCCAGAACGTGAACAAGTTTCT
GAACTGGTATCAGCAGAAGCCCGGCAAGGCCCCTAGGAGGCTGATCTAC
GGCACAAATTCTCTGCAGAGCGGCGTGCCTAGCAGATTTAGCGGCAGCG
GAAGCGGCACTGACTTCACACTGACTATCAGCTCTCTGCAGCCAGAGGA
CTTCGCCACATACTACTGCCAGCAGTACACAAGCTGGCCTTGGACATTC
GGCCAAGGCACAAAGCTGGAGATCAAG

In one embodiment, the nucleic acid according to the present invention comprises or consists of:
a sequence encoding the VH of the antibody or antigen-binding fragment thereof according to the present invention; and
a sequence encoding the VL of the antibody or antigen-binding fragment thereof according to the present invention.

In one embodiment, the nucleic acid according to the present invention comprises or consists of:
a sequence selected from the group comprising or consisting of SEQ ID NO: 29, SEQ ID NO: 30, SEQ ID NO: 31, SEQ ID NO: 32, SEQ ID NO: 33 and any sequence sharing at least 70%, 75%, 80%, 85%, 90%, 95%, 96%, 97%, 98%, 99% or more identity with SEQ ID NO: 29-33; and
a sequence selected from the group comprising or consisting of SEQ ID NO: 34, SEQ ID NO: 35, SEQ ID NO: 36 and any sequence sharing at least 70%, 75%, 80%, 85%, 90%, 95%, 96%, 97%, 98%, 99% or more identity with SEQ ID NO: 34-36,
with the proviso that
when the nucleic acid comprises a sequence SEQ ID NO: 29 or SEQ ID NO: 30, the nucleic acid does not comprise a sequence SEQ ID NO: 34.

In one embodiment, the nucleic acid according to the present invention comprises or consists of:
a sequence encoding the VH comprising or consisting of the sequence SEQ ID NO: 29; and
a sequence encoding the VL comprising or consisting of the sequence SEQ ID NO: 35.

In one embodiment, said nucleic acid encodes for the VH and VL of the MAB02 antibody.

In one embodiment, the nucleic acid according to the present invention comprises or consists of:
a sequence encoding the VH comprising or consisting of the sequence SEQ ID NO: 29; and
a sequence encoding the VL comprising or consisting of the sequence SEQ ID NO: 36.

In one embodiment, said nucleic acid encodes for the VH and VL of the MAB03 antibody.

In one embodiment, the nucleic acid according to the present invention comprises or consists of:
a sequence encoding the VH comprising or consisting of the sequence SEQ ID NO: 30; and
a sequence encoding the VL comprising or consisting of the sequence SEQ ID NO: 35.

In one embodiment, said nucleic acid encodes for the VH and VL of the MAB06 antibody.

In one embodiment, the nucleic acid according to the present invention comprises or consists of:
a sequence encoding the VH comprising or consisting of the sequence SEQ ID NO: 30; and
a sequence encoding the VL comprising or consisting of the sequence SEQ ID NO: 36.

In one embodiment, said nucleic acid encodes for the VH and VL of the MAB07 antibody

In one embodiment, the nucleic acid according to the present invention comprises or consists of:
a sequence encoding the VH comprising or consisting of the sequence SEQ ID NO: 31; and
a sequence encoding the VL comprising or consisting of the sequence SEQ ID NO: 34.

In one embodiment, said nucleic acid encodes for the VH and VL of the MAB09 antibody.

In one embodiment, the nucleic acid according to the present invention comprises or consists of:
a sequence encoding the VH comprising or consisting of the sequence SEQ ID NO: 31; and
a sequence encoding the VL comprising or consisting of the sequence SEQ ID NO: 35.

In one embodiment, said nucleic acid encodes for the VH and VL of the MAB10 antibody.

In one embodiment, the nucleic acid according to the present invention comprises or consists of:
a sequence encoding the VH comprising or consisting of the sequence SEQ ID NO: 31; and
a sequence encoding the VL comprising or consisting of the sequence SEQ ID NO: 36.

In one embodiment, said nucleic acid encodes for the VH and VL of the MAB11 antibody.

In one embodiment, the nucleic acid according to the present invention comprises or consists of:
a sequence encoding the VH comprising or consisting of the sequence SEQ ID NO: 32; and
a sequence encoding the VL comprising or consisting of the sequence SEQ ID NO: 34.

In one embodiment, said nucleic acid encodes for the VH and VL of the MAB13 antibody.

In one embodiment, the nucleic acid according to the present invention comprises or consists of:
a sequence encoding the VH comprising or consisting of the sequence SEQ ID NO: 32; and
a sequence encoding the VL comprising or consisting of the sequence SEQ ID NO: 35.

In one embodiment, said nucleic acid encodes for the VH and VL of the MAB14 antibody.

In one embodiment, the nucleic acid according to the present invention comprises or consists of:
a sequence encoding the VH comprising or consisting of the sequence SEQ ID NO: 32; and
a sequence encoding the VL comprising or consisting of the sequence SEQ ID NO: 36.

In one embodiment, said nucleic acid encodes for the VH and VL of the MAB15 antibody.

In one embodiment, the nucleic acid according to the present invention comprises or consists of:
a sequence encoding the VH comprising or consisting of the sequence SEQ ID NO: 33; and
a sequence encoding the VL comprising or consisting of the sequence SEQ ID NO: 34.

In one embodiment, said nucleic acid encodes for the VH and VL of the MAB17 antibody.

In one embodiment, the nucleic acid according to the present invention comprises or consists of:
a sequence encoding the VH comprising or consisting of the sequence SEQ ID NO: 33; and
a sequence encoding the VL comprising or consisting of the sequence SEQ ID NO: 35.

In one embodiment, said nucleic acid encodes for the VH and VL of the MAB18 antibody.

In one embodiment, the nucleic acid according to the present invention comprises or consists of:
a sequence encoding the VH comprising or consisting of the sequence SEQ ID NO: 33; and
a sequence encoding the VL comprising or consisting of the sequence SEQ ID NO: 36.

In one embodiment, said nucleic acid encodes for the VH and VL of the MAB19 antibody.

In one embodiment, the VH and/or the VL further comprises a leader sequence, preferably located in the 5′ from the VH nucleic acid sequence or in the 5′ from the VL nucleic acid sequence, respectively. Examples of leader sequences include, but are not limited to, SEQ ID NO: 27 and 28, encoded respectively by SEQ ID NO: 37 and SEQ ID NO: 38.

SEQ ID NO: 37
ATGGACATCAGGCTCAGCTTGGCTTTCCTTGTCCTTTTCATAAAAGGTG
TCCAGTGT
SEQ ID NO: 38
ATGGCTGCAGTTCAACTCTTAGGGCTGCTGCTGCTTTGGCTCCCAGCCA
TGAGATGT

In one embodiment, the VH comprises a nucleic acid leader sequence SEQ ID NO: 37 located in the 5′ from the VH-encoding nucleic acid sequence (e.g., SEQ ID NO: 29, SEQ ID NO: 30, SEQ ID NO: 31, SEQ ID NO: 32 or SEQ ID NO: 33).

In one embodiment, the VL comprises a nucleic acid sequence leader sequence SEQ ID NO: 38 located in the 5′ from the VL-encoding nucleic acid sequence (e.g., SEQ ID NO: 34, SEQ ID NO: 35 or SEQ ID NO: 36).

In one embodiment, the nucleic acid according to the present invention comprises a sequence encoding a fully or substantially fully human C_{H} and/or C_{L} of the antibody or antigen-binding fragment thereof according to the present invention. In such embodiment, constant regions may be derived from any human antibody constant regions.

In one embodiment, the nucleic acid according to the present invention comprises a sequence encoding a fully or substantially fully murine C_{H} and/or C_{L} of the antibody or antigen-binding fragment thereof according to the invention. In such embodiment, constant regions may be derived from any murine antibody constant regions.

In one embodiment, the nucleic acid according to the present invention comprises or consists of a sequence encoding the heavy chain of the chimeric antibody or antigen-binding fragment thereof according to the invention. In one embodiment, the nucleic acid according to the present invention comprises or consists of a sequence encoding the light chain of the chimeric antibody or antigen-binding fragment thereof according to the invention.

In one embodiment, the nucleic acid according to the present invention comprises or consists of a sequence encoding the heavy chain of the humanized antibody or antigen-binding fragment thereof according to the invention. In one embodiment, the nucleic acid according to the present invention comprises or consists of a sequence encoding the light chain of the humanized antibody or antigen-binding fragment thereof according to the invention.

Typically, said nucleic acid is a DNA or RNA molecule, which may be included in any suitable vector, such as for example plasmid, cosmid, episome, artificial chromosome, phage or a viral vector.

Thus, another object of the present invention is an expression vector comprising a nucleic acid encoding the protein, antibody or antigen-binding fragment thereof according to the present invention. Another object of the present invention is an expression vector comprising a nucleic acid encoding a fusion protein according to the present invention.

The terms “vector”, “cloning vector” and “expression vector” mean the vehicle by which a DNA or RNA sequence (e.g. a foreign gene) can be introduced into a host cell, so as to transform a host and promote expression (e.g. transcription and translation) of the introduced sequence. Such vectors may comprise regulatory elements, such as a promoter, enhancer, terminator and the like, to cause or direct expression of said protein or antibody or antigen-binding fragment thereof or fusion protein upon administration to a host. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the protein, antibody or fragment thereof or fusion protein can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found in the art.

In one embodiment, the expression vector according to the present invention comprises a sequence encoding the VH of the antibody or antigen-binding fragment thereof according to the invention, operably linked to regulatory elements.

In one embodiment, the expression vector according to the present invention comprises a sequence SEQ ID NO: 29, SEQ ID NO: 30, SEQ ID NO: 31, SEQ ID NO: 32, SEQ ID NO: 33 or any sequence sharing at least 70%, 75%, 80%, 85%, 90%, 95%, 96%, 97%, 98%, 99% or more identity with SEQ ID NO: 29-33, operably linked to regulatory elements.

In one embodiment, the expression vector according to the present invention comprises a sequence encoding the VL of the antibody or antigen-binding fragment thereof according to the invention, operably linked to regulatory elements.

In one embodiment, the expression vector according to the present invention comprises a sequence SEQ ID NO: 34, SEQ ID NO: 35, SEQ ID NO: 36 or any sequence sharing at least 70%, 75%, 80%, 85%, 90%, 95%, 96%, 97%, 98%, 99% or more identity with SEQ ID NO: 34-36, operably linked to regulatory elements.

In one embodiment, the expression vector according to the present invention comprises:
a sequence encoding the VH of the antibody or antigen-binding fragment thereof according to the invention, operably linked to regulatory elements; and
a sequence encoding the VL of the antibody or antigen-binding fragment thereof according to the invention, operably linked to regulatory elements.

In one embodiment, the expression vector according to the present invention comprises:
a sequence encoding the VH comprising or consisting of the sequence SEQ ID NO: 29, SEQ ID NO: 30, SEQ ID NO: 31, SEQ ID NO: 32, SEQ ID NO: 33 or any sequence sharing at least 70%, 75%, 80%, 85%, 90%, 95%, 96%, 97%, 98%, 99% or more identity with SEQ ID NO: 29-33, operably linked to regulatory elements, and
a sequence encoding the VL comprising or consisting of the sequence SEQ ID NO: 34, SEQ ID NO: 35, SEQ ID NO: 36 or any sequence sharing at least 70%, 75%, 80%, 85%, 90%, 95%, 96%, 97%, 98%, 99% or more identity with SEQ ID NO: 34-36, operably linked to regulatory elements,
with the proviso that
when the expression vector comprises a sequence encoding the VH comprising or consisting of the sequence SEQ ID NO: 29 or SEQ ID NO: 30 operably linked to regulatory elements, the expression vector does not comprise a sequence encoding the VL comprising or consisting of the sequence SEQ ID NO: 34 operably linked to regulatory elements.

In one embodiment, the expression vector according to the present invention comprises:
a sequence encoding the VH comprising or consisting of the sequence SEQ ID NO: 29, operably linked to regulatory elements; and
a sequence encoding the VL comprising or consisting of the sequence SEQ ID NO: 35, operably linked to regulatory elements.

In one embodiment, the expression vector according to the present invention comprises:
a sequence encoding the VH comprising or consisting of the sequence SEQ ID NO: 29, operably linked to regulatory elements; and
a sequence encoding the VL comprising or consisting of the sequence SEQ ID NO: 36, operably linked to regulatory elements.

In one embodiment, the expression vector according to the present invention comprises:
a sequence encoding the VH comprising or consisting of the sequence SEQ ID NO: 30, operably linked to regulatory elements; and
a sequence encoding the VL comprising or consisting of the sequence SEQ ID NO: 35, operably linked to regulatory elements.

In one embodiment, the expression vector according to the present invention comprises:
a sequence encoding the VH comprising or consisting of the sequence SEQ ID NO: 30, operably linked to regulatory elements; and
a sequence encoding the VL comprising or consisting of the sequence SEQ ID NO: 36, operably linked to regulatory elements.

In one embodiment, the expression vector according to the present invention comprises:
a sequence encoding the VH comprising or consisting of the sequence SEQ ID NO: 31, operably linked to regulatory elements; and
a sequence encoding the VL comprising or consisting of the sequence SEQ ID NO: 34, operably linked to regulatory elements.

In one embodiment, the expression vector according to the present invention comprises:
a sequence encoding the VH comprising or consisting of the sequence SEQ ID NO: 31, operably linked to regulatory elements; and
a sequence encoding the VL comprising or consisting of the sequence SEQ ID NO: 35, operably linked to regulatory elements.

In one embodiment, the expression vector according to the present invention comprises:
a sequence encoding the VH comprising or consisting of the sequence SEQ ID NO: 31, operably linked to regulatory elements; and
a sequence encoding the VL comprising or consisting of the sequence SEQ ID NO: 36, operably linked to regulatory elements.

In one embodiment, the expression vector according to the present invention comprises:
a sequence encoding the VH comprising or consisting of the sequence SEQ ID NO: 32, operably linked to regulatory elements; and
a sequence encoding the VL comprising or consisting of the sequence SEQ ID NO: 34, operably linked to regulatory elements.

In one embodiment, the expression vector according to the present invention comprises:
a sequence encoding the VH comprising or consisting of the sequence SEQ ID NO: 32, operably linked to regulatory elements; and
a sequence encoding the VL comprising or consisting of the sequence SEQ ID NO: 35, operably linked to regulatory elements.

In one embodiment, the expression vector according to the present invention comprises:
a sequence encoding the VH comprising or consisting of the sequence SEQ ID NO: 32, operably linked to regulatory elements; and
a sequence encoding the VL comprising or consisting of the sequence SEQ ID NO: 36, operably linked to regulatory elements.

In one embodiment, the expression vector according to the present invention comprises:
a sequence encoding the VH comprising or consisting of the sequence SEQ ID NO: 33, operably linked to regulatory elements; and
a sequence encoding the VL comprising or consisting of the sequence SEQ ID NO: 34, operably linked to regulatory elements.

In one embodiment, the expression vector according to the present invention comprises:
a sequence encoding the VH comprising or consisting of the sequence SEQ ID NO: 33, operably linked to regulatory elements; and
a sequence encoding the VL comprising or consisting of the sequence SEQ ID NO: 35, operably linked to regulatory elements.

In one embodiment, the expression vector according to the present invention comprises:
a sequence encoding the VH comprising or consisting of the sequence SEQ ID NO: 33, operably linked to regulatory elements; and
a sequence encoding the VL comprising or consisting of the sequence SEQ ID NO: 36, operably linked to regulatory elements.

In one embodiment, the expression vector according to the present invention comprises a sequence encoding the C_{H} of the antibody or antigen-binding fragment thereof according to the invention, operably linked to regulatory elements, wherein said C_{H} may be derived from any human antibody C_{H}.

In one embodiment, the expression vector according to the present invention comprises a sequence encoding the C_{L} of the antibody or antigen-binding fragment thereof according to the invention, operably linked to regulatory elements, wherein said C_{L} may be derived from any human antibody C_{L}.

In one embodiment, the expression vector according to the present invention comprises a sequence encoding the C_{H} of the antibody or antigen-binding fragment thereof according to the invention, operably linked to regulatory elements, wherein said C_{H} may be derived from any murine antibody C_{H}.

In one embodiment, the expression vector according to the present invention comprises a sequence encoding the C_{L} of the antibody or antigen-binding fragment thereof according to the invention, operably linked to regulatory elements, wherein said C_{L} may be derived from any murine antibody C_{L}.

In one embodiment, the expression vector according to the present invention comprises a sequence encoding the heavy chain of the chimeric antibody or antigen-binding fragment thereof according to the present invention, operably linked to regulatory elements.

In one embodiment, the expression vector according to the present invention comprises a sequence encoding the light chain of the chimeric antibody or antigen-binding fragment thereof according to the present invention, operably linked to regulatory elements.

In one embodiment, the expression vector according to the present invention comprises a sequence encoding the heavy chain of the humanized antibody or antigen-binding fragment thereof according to the present invention, operably linked to regulatory elements.

In one embodiment, the expression vector according to the present invention comprises a sequence encoding the light chain of the humanized antibody or antigen-binding fragment thereof according to the present invention, operably linked to regulatory elements.

In one embodiment, the expression vector according to the present invention is monocistronic.

By “monocistronic”, it is meant that a single nucleic acid is expressed in a single expression vector.

In one embodiment, the expression vector according to the present invention is polycistronic.

By “polycistronic”, it is meant that at least two or more nucleic acids are expressed in a single expression vector.

Another object of the invention is an isolated host cell comprising said vector. Said host cell may be used for the recombinant production of the proteins, antibodies or antigen-binding fragments thereof, or the fusion proteins of the invention.

In an embodiment, host cells may be prokaryote, yeast, or eukaryote cells, preferably mammalian cells, such as, for example: monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse Sertoli cells (TM4, Mather, Biol. Reprod. 23:243-251 (1980)); mouse myeloma cells SP2/0-AG14 (ATCC CRL 1581; ATCC CRL 8287) or NSO (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art. It should be noted that the term “host cell” generally refers to a cultured cell line. In one embodiment, whole human beings into which an expression vector encoding a protein, an antibody or an antigen-binding fragment thereof, or a fusion protein according to the invention has been introduced are excluded from the definition of a “host cell”.

Another object of the present invention is a method of producing and purifying the isolated protein of the invention, in particular the antibody or an antigen-binding fragment thereof as described herein.

In one embodiment, the method comprises:
introducing in vitro or ex vivo a recombinant nucleic acid or a vector as described hereinabove into a competent host cell;
culturing in vitro or ex vivo the host cells transformed with the nucleic acid or expression vector, under conditions suitable for expression of the protein of the invention, in particular of the antibody or antigen-binding fragment thereof;
optionally, selecting the cells which express and/or secrete said protein; and
recovering the expressed protein (in particular the expressed antibody or antigen-binding fragment thereof).

This recombinant process can be used for large scale production of proteins, such as, for example, of antibodies or antigen-binding fragments thereof, including monoclonal antibodies intended for in vitro, ex vivo and/or in vivo therapeutic uses.

In one embodiment, the expressed protein, in particular the expressed antibody or antigen-binding fragment thereof, is further purified.

Methods to purify a protein, in particular an antibody or antigen-binding fragment thereof are well-known in the art and include, without limitation, protein A-Sepharose, gel electrophoresis, chromatography, preferably by affinity chromatography, more preferably by affinity chromatography on protein L agarose.

Another object of the present invention is a composition comprising, consisting essentially of or consisting of at least one protein binding to human CD25 (hCD25) according to the present invention, in particular at least one antibody binding to human CD25 (hCD25) as described herein or at least one antigen-binding fragment of said antibody.

Another object of the present invention is a composition comprising, consisting essentially of or consisting of at least one fusion protein according to the present invention.

Another object of the present invention is a composition comprising, consisting essentially of or consisting of at least one nucleic acid encoding a protein, an antibody or an antigen-binding fragment of said antibody, or a fusion protein according to the present invention.

Another object of the present invention is a composition comprising, consisting essentially of or consisting of at least one expression vector comprising at least one nucleic acid encoding a protein, an antibody or an antigen-binding fragment of said antibody or a fusion protein according to the present invention.

Another object of the present invention is a pharmaceutical composition comprising, consisting essentially of or consisting of at least one protein binding to hCD25 according to the present invention, in particular at least one antibody binding to human CD25 (hCD25) as described herein or at least one antigen-binding fragment of said antibody, and at least one pharmaceutically acceptable excipient.

Another object of the present invention is a pharmaceutical composition comprising, consisting essentially of or consisting of at least one fusion protein according to the present invention and at least one pharmaceutically acceptable excipient.

Another object of the present invention is a pharmaceutical composition comprising, consisting essentially of or consisting of at least one nucleic acid encoding a protein of the present invention, in particular encoding an antibody binding to human CD25 (hCD25) as described herein or an antigen-binding fragment of said antibody, or a fusion protein according to the present invention and at least one pharmaceutically acceptable excipient.

Another object of the present invention is a pharmaceutical composition comprising, consisting essentially of or consisting of at least one expression vector comprising at least one nucleic acid encoding a protein of the present invention, in particular encoding an antibody binding to human CD25 (hCD25) as described herein or an antigen-binding fragment of said antibody or a fusion protein according to the present invention, and at least one pharmaceutically acceptable excipient.

As used herein, “consisting essentially of”, with reference to a composition, means that the at least one protein, antibody or antigen-binding fragment thereof, fusion protein, nucleic acid or expression vector is the only one therapeutic agent or agent with a biologic activity within said composition.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a mammal, more preferably a human. For human administration, preparations should meet sterility, pyrogenicity, and general safety and purity standards as required by regulatory offices, such as, for example, FDA Office or EMA.

Examples of pharmaceutically acceptable excipients that may be used in the compositions of the present invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Another object of the present invention is a medicament comprising, consisting essentially of or consisting of at least one protein binding to hCD25 according to the present invention, in particular at least one antibody binding to human CD25 (hCD25) as described herein or at least one antigen-binding fragment of said antibody.

Another object of the present invention is a medicament comprising, consisting essentially of or consisting of at least one fusion protein according to the present invention.

Another object of the present invention is a medicament comprising, consisting essentially of or consisting of at least one nucleic acid encoding a protein of the present invention, in particular encoding an antibody binding to human CD25 (hCD25) as described herein or an antigen-binding fragment of said antibody or a fusion protein according to the present invention.

Another object of the present invention is a medicament comprising, consisting essentially of or consisting of at least one expression vector comprising at least one nucleic acid encoding a protein of the present invention, in particular encoding an antibody binding to human CD25 (hCD25) as described herein or an antigen-binding fragment of said antibody or a fusion protein according to the present invention.

For use in administration to a subject, the composition, pharmaceutical composition or medicament will be formulated for administration to the subject.

In one embodiment, the composition, pharmaceutical composition or medicament according to the present invention is administered (or is to be administered) parenterally, by inhalation spray, rectally, nasally, or via an implanted reservoir.

In one embodiment, the composition, pharmaceutical composition or medicament is administered (or is to be administered) by injection, including, without limitation, subcutaneous, intravenous, intramuscular, intra-articular, intra-synovial, intrasternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Sterile injectable forms of the compositions, pharmaceutical compositions or medicaments of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one embodiment, the isolated protein, the isolated antibody or antigen-binding fragment thereof, the fusion protein, nucleic acid, expression vector, composition, pharmaceutical composition or medicament according to the present invention is to be administered to the subject in need thereof in a therapeutically effective amount.

It will be however understood that the total daily usage of the isolated protein, isolated antibody or antigen-binding fragment thereof, fusion protein, nucleic acid, expression vector, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the isolated protein, isolated antibody or antigen-binding fragment thereof, fusion protein, nucleic acid, or expression vector employed; the age, body weight, general health, sex and diet of the subject; the time of administration, route of administration, and rate of excretion of the specific isolated protein, isolated antibody or antigen-binding fragment thereof, fusion protein, nucleic acid, or expression vector employed; the duration of the treatment; drugs used in combination or coincidental with the specific isolated protein, isolated antibody or antigen-binding fragment thereof, fusion protein, nucleic acid, or expression vector employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

The daily dosage of the proteins, antibodies or antigen-binding fragments thereof, fusion proteins, nucleic acids or expression vectors may be varied over a wide range from about 0.01 to about 1000 mg per adult per day. Compositions may contain about 0.01, 0.05, 0.1, 0.5, 1.0, 2.5, 5.0, 10.0, 15.0, 25.0, 50.0, 100, 250, and about 500 mg of the active ingredient for the symptomatic adjustment of the dosage to the subject to be treated. A pharmaceutical composition or medicament typically contains from about 0.01 mg to about 500 mg of active ingredient. A therapeutically effective amount of the drug is ordinarily supplied at a dosage level from about 0.0002 mg/kg to about 20 mg/kg of body weight per day. For example, a protein, an antibody or antigen-binding fragment thereof, a fusion protein, a nucleic acid or an expression vector present in a composition, pharmaceutical composition or medicament of this invention can be supplied at a concentration ranging from about 1 mg/mL to about 100 mg/mL, such as, for example, at a concentration of about 1 mg/mL, 5 mg/mL, 10 mg/mL, 50 mg/mL or about 100 mg/mL. In one embodiment, the protein, antibody or antigen-binding fragment thereof, fusion protein, nucleic acid or expression vector is supplied at a concentration of about 10 mg/mL in either 100 mg (10 mL) or 500 mg (50 mL) single use-vials. It will be appreciated that these dosages are exemplary and that an optimal dosage can be adapted taking into account the affinity and tolerability of the particular protein, antibody or antigen-binding fragment, fusion protein, nucleic acid or expression vector that must be determined in clinical trials.

In one embodiment, the proteins, antibodies or antigen-binding fragments thereof, fusion proteins, nucleic acids or expression vectors of the present invention are to be administered at a dosage level of about 0.01 mg, 0.05 mg, 0.1 mg, 0.5 mg, 1 mg, 5 mg, 10 mg, 50 mg, 100 mg, 500 mg, or of about 1000 mg per adult per day.

The present invention relates to at least one isolated protein as described herein, in particular to at least one antibody binding to human CD25 (hCD25) as described herein or to at least one antigen-binding fragment of said antibody, for use as a medicament, i.e., for treating (or for use in treating) diseases, disorders or symptoms in a subject in need thereof.

The present invention relates to at least one fusion protein as described herein, for use as a medicament, i.e., for treating (or for use in treating) diseases, disorders or symptoms in a subject in need thereof.

The present invention relates to at least one nucleic acid as described herein, for use as a medicament, i.e., for treating (or for use in treating) diseases, disorders or symptoms in a subject in need thereof.

The present invention relates to at least one expression vector as described herein, for use as a medicament, i.e., for treating (or for use in treating) diseases, disorders or symptoms in a subject in need thereof.

The present invention relates to a composition, pharmaceutical composition, or a medicament as described hereinabove, for use in treating diseases, disorders or symptoms in a subject in need thereof.

The present invention thus further relates to a method for treating diseases, disorders or symptoms in a subject in need thereof, comprising administering to the subject an isolated protein (in particular an antibody or antigen-binding fragment thereof), a fusion protein, a nucleic acid or a vector, or a composition, a pharmaceutical composition, or a medicament as described herein.

Examples of diseases that may be treated in the present invention, include, but are not limited to, cancers and infectious diseases.

In one embodiment, the isolated protein, the antibody or antigen-binding fragment thereof, the fusion protein, the nucleic acid or the vector according to the present invention may be used for treating cancer in a subject in need thereof.

In one embodiment, a therapeutically effective amount of said protein, antibody or antigen-binding fragment thereof, fusion protein, nucleic acid or expression vector is administered or is to be administered to the subject.

As used herein, the term “cancer” has its general meaning in the art and includes, but is not limited to, solid tumors and blood borne tumors. The term cancer includes, without limitation, diseases of the skin, tissues, organs, bone, cartilage, blood and vessels. The term “cancer” further encompasses both primary and metastatic cancers.

Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, endometrial, pancreas or uterus.

In addition, the cancer may be selected in the following non-limiting list: malignant neoplasm; undifferentiated carcinoma; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; malignant gastrinoma; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma associated with familial polyposis coli; solid carcinoma; malignant carcinoid tumor; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease of the breast; acinar cell carcinoma; adenosquamous carcinoma; adenocarcinoma with squamous metaplasia; malignant thymoma; malignant ovarian stromal tumor; malignant thecoma; malignant granulosa cell tumor; malignant roblastoma; Sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malign melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; malignant lymphoma—small lymphocytic; malignant diffuse large cell lymphoma; malignant follicular lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In another embodiment, the isolated protein, antibody or antigen-binding fragment thereof, fusion protein, nucleic acid or expression vector according to the present invention may be used in the treatment of an infectious disease, disorder or symptom thereof in a subject in need thereof.

In one embodiment, a therapeutically effective amount of a protein, of an antibody or antigen-binding fragment thereof, of a fusion protein, of a nucleic acid or of an expression vector of the present invention is administered or is to be administered to the subject.

As used herein the term “infectious disease” includes any infection caused by viruses, bacteria, protozoa, molds or fungi.

In some embodiments, the viral infection comprises infection by one or more viruses selected from the group comprising, but not limited to, Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses.

In some embodiments, the viral infection comprises infection by one or more viruses selected from the group comprising, but not limited to, adenovirus, Alfuy virus, Banzi virus, bovine diarrhea virus, coronavirus, Coxsackie virus, Crimean-Congo virus, Dengue virus, Ebola virus, encephalitis viruses (including Japanese Encephalitis virus, California Encephalitis virus, Venezuelan equine encephalitis virus, Western equine encephalitis virus, Eastern equine encephalitis virus, St. Louis encephalitis virus, tick-borne encephalitis virus), guanarito virus, hantavirus, hepatitis virus, Ileus virus, immunodeficiency virus, influenza viruses including influenza A and influenza B viruses (including human, avian, and swine) and parainfluenza virus, junin virus, Kokobera virus, Kunjin virus, Kyasanur Forest disease virus, La Crosse virus, Lassa virus, louping-ill virus, lymphocytic choriomeningitis virus, measles virus, machupo virus, Marburg virus, Murray Valley virus, pachindae viruses, Pichinde virus, poliovirus, Powassan virus, Punta Toro virus, respiratory syncytial virus, rhinovirus, Rift Valley Fever virus, Rocio virus, severe acute respiratory syndrome (SARS), small pox virus, Tacaribe virus, West Nile and yellow fever viruses.

Examples of bacterial infections that may be treated in the present invention include, but are not limited to, infections caused by the following: Staphylococcus; Streptococcus, including S. pyogenes; Enterococci; Bacillus, such as, for example Bacillus anthracis, and Lactobacillus; Listeria; Corynebacterium diphtheriae; Gardnerella such as, for example G. vaginalis; Nocardia; Streptomyces; Thermoactinomyces vulgaris; Treponerna; Campylobacter, Pseudomonas such as, for example, P. aeruginosa; Legionella; Neisseria such as, for example N. gonorrhoeae and N. meningitides; Flavobacterium such as, for example F. meningosepticum and F. odoraturn; Brucella; Bordetella such as, for example B. pertussis and B. bronchiseptica; Escherichia such as, for example E. coli, Klebsiella; Enterobacter, Serratia such as, for example S. marcescens and S. liquefaciens; Edwardsiella; Proteus such as, for example P. mirabilis and P. vulgaris; Streptobacillus; Rickettsiaceae such as, for example R. rickettsii, Chlamydia such as, for example C. psittaci and C. trachornatis; Mycobacterium such as, for example M. tuberculosis, M. intracellulare, M. folluiturn, M. laprae, M. avium, M. bovis, M. africanum, M. kansasii, and M. lepraernurium; and Nocardia.

Examples of protozoa infections that may be treated in the present invention include, but are not limited to, infections caused by leishmania, kokzidioa, and trypanosoma.

A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference. All of said diseases are candidates for treatment using the isolated protein, antibody or antigen-binding fragment thereof, fusion protein, nucleic acid, expression vector, composition, pharmaceutical composition or medicament according to the invention.

In one embodiment, the isolated protein (in particular the antibody or antigen-binding fragment thereof), fusion protein, nucleic acid, or expression vector according to the present invention is used alone.

In another embodiment, the isolated protein (in particular the antibody or antigen-binding fragment thereof), fusion protein, nucleic acid, or expression vector according to the present invention is used in combination with at least one further therapeutic agent.

In one embodiment, the administration of the at least one further therapeutic agent and of the isolated protein (in particular the antibody or antigen-binding fragment thereof), fusion protein, nucleic acid, or expression vector according to the present invention is simultaneous, separate or sequential.

In one embodiment, for simultaneous administration, the at least one further therapeutic agent and the isolated protein (in particular the antibody or antigen-binding fragment thereof), fusion protein, nucleic acid, or expression vector according to the present invention are administered as one composition or as separate compositions, as appropriate.

Examples of additional therapeutic agents include, but are not limited to, chemotherapeutic agents, targeted cancer therapy, radiotherapy, immunotherapeutic agents or anti-cancer immunogens, anti-cancer antibodies, cytotoxic agents, anti-angiogenic agents, cell cycle control/apoptosis regulating agents, hormonal regulating agents, and other immunosuppressive and/or anti-inflammatory drugs selected from corticoids, such as, for example, glucocorticoids.

In one embodiment, the at least one further therapeutic agent is a therapeutic agent useful for treating the specific disease, disorder or condition to be treated in the present invention. For example, for treating cancer, the at least one further therapeutic agent may be selected from the group comprising, but not limited to, chemotherapeutic agents, targeted cancer therapy, radiotherapy, immunotherapeutic agents or anti-cancer immunogens, anti-cancer antibodies, cytotoxic agents, anti-angiogenic agents, cell cycle control/apoptosis regulating agents, hormonal regulating agents, and other immunosuppressive and/or anti-inflammatory drugs selected from corticoids, such as, for example, glucocorticoids.

In one embodiment, the isolated protein (in particular the antibody or antigen-binding fragment thereof as described herein), fusion protein, nucleic acid, or expression vector according to the present invention is used in combination with a chemotherapeutic agent.

The term “chemotherapeutic agent” refers to chemical compounds that are effective in inhibiting tumor growth.

Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredepa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin 11 and calicheamicin 211; dynemicin, including dynemicin A; an esperamicin; neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylamine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above.

Also included in the definition of chemotherapeutic agents are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In one embodiment, the isolated protein, antibody or antigen-binding fragment thereof, fusion protein, nucleic acid, or vector according to the present invention is used in combination with a targeted cancer therapy.

As used herein, the term “targeted cancer therapies” are drugs or other substances that block the growth and spread of cancer by interfering with specific molecules (“molecular targets”) that are involved in the growth, progression, and spread of cancer. Targeted cancer therapies are sometimes called “molecularly targeted drugs”, “molecularly targeted therapies”, “precision medicines”. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, ABT-869, AEE-788, AEW-541, Axitinib, AZM-475271, BEZ235, BMS-599626 (AC-480), Bosutinib, Brivanib (BMS-582664), canertinib (CI 1033), Cediranib, CEP-11981, CP-547632, CP-724714, dasatinib (BMS-354825), Dovitinib, Enzastaurin, erlotinib (Tarceva; OSI-1774), gefitinib (Iressa), imatinib (Gleevec; STI571), KRN-633, KRN-951, lapatinib (GW572016; GW2016), leflunomide (SU101), Lestaurtinib, L-21649, Motesanib, Midostaurin, MKC-I (Ro-317453; R-440), MK-2206 (8-[4-aminocyclobutyl)phenyl]-9-phenyl-1,2,4-triazolo[3,4-f][1,6]naphthyridin-3(2H)-one hydrochloride), MLN-8054, Neratinib, Nilotinib, OSI-930, Pazopanib, PD-0325901, PD-0332991, PP2, saracatinib, semaxanib (SU5416), Seliciclib, SNS-032, sorafenib (BAY 43-9006), sunitinib (Sutent; SU11248), SU-14813, SU-6668 (TSU-68), TAK-165, Tandutinib, Telatinib, vatalanib (PTK787/ZK222584), vandetanib (Zactima; ZD6474), derivatives thereof, analogs thereof, and combinations thereof.

In one embodiment, the isolated protein, antibody or antigen-binding fragment thereof, fusion protein, nucleic acid, or vector according to the present invention is used in combination with radiotherapy.

The term “radiotherapy” may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In one embodiment, the isolated protein, antibody or antigen-binding fragment thereof, fusion protein, nucleic acid or expression vector according to the present invention is used in combination with an immunotherapeutic agent or immunotherapy.

The terms “immunotherapeutic agent” or “immunotherapy” as used herein, refer to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy, biological response modifier therapy and biotherapy. Examples of common immunotherapeutic agents or immunotherapies known in the art include, but are not limited to: cytokines, checkpoint inhibitors, checkpoint agonists also referred to as T cell agonists, antibodies including monoclonal antibodies, antibody domains, antibody fragments, bispecific antibodies, preventive and therapeutic vaccines, oncolytic viruses, adoptive transfer of immune cells (T cells, NK, cells, dendritic cells, B cells . . . ).

One of the central premises underlying cancer immunotherapy is the presence of antigens which are selectively or abundantly expressed or mutated in cancer cells, thus enabling the specific recognition and subsequent destruction of the cancer cells. Such antigens are commonly referred to as tumor-specific antigens. Another of the central premises underlying cancer immunotherapy is the presence of lymphocytes in the tumors, i.e., tumor infiltrating lymphocytes (TILs), and notably of effector TILs which can target and kill the tumor cells through the recognition of the above-mentioned tumor-specific antigens.

Immunotherapeutic agents or therapies can be passive. A passive immunotherapeutic agent is one that produces an immediate action due to the administration of immune-cell factors, like monoclonal antibodies. The results of a passive immunotherapy are tied temporally to administration of the agent, therefore continued dosing may be required for a prolonged response. In another embodiment, the immunotherapeutic agent or therapies are active. An active immunotherapeutic agent is one that produces a lasting, durable response by way of inducing immunological memory. This most closely resembles a normal immune response. However, just as immune system function varies in a healthy population, the level of response to an active immunotherapy agent depends on individual factors.

Active immunotherapeutic agents include both non-specific active agents (i.e., agents that boost the immune system generally so that the human body becomes more effective in fighting the growth and/or spread of cancer cells), and specific active agents, (i.e., agents inducing the generation of cell-mediated and antibody immune responses focused on specific antigens expressed by the cancer cells). Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g., cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

In one embodiment, the isolated protein, antibody or antigen-binding fragment thereof, fusion protein, nucleic acid or vector according to the present invention is used in combination with a cytokine therapy.

As used herein, a “cytokine therapy” is defined as the administration of at least one cytokine to the subject.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behavior and/or increasing their production of antigens thus making the cancer cells easier for the immune system to recognize and destroy. IFNs can also act indirectly on cancer cells, for example, by slowing down angiogenesis, boosting the immune system and/or stimulating natural killer (NK) cells, T cells and macrophages. Recombinant TFN-alpha is available commercially as Roferon (Roche Pharmaceuticals) and Intron A (Schering Corporation). Interleukins contemplated by the present invention include IL-2, IL-4, IL-11, IL-12 and IL-21. Examples of commercially available recombinant interleukins include Proleukin® (IL-2; Chiron Corporation) and Neumega® (IL-12; Wyeth Pharmaceuticals). Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoetin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pegfilgrastim; Amgen), Leukine (GM-CSF; Berlex), Procrit (erythropoietin; Ortho Biotech), Epogen (erythropoietin; Amgen), Aranesp (erythropoietin).

In one embodiment, the isolated protein, antibody or antigen-binding fragment thereof, fusion protein, nucleic acid or vector according to the present invention is used in combination with a cytokine mimetics, such as, for example, an IL-2 mimetics. In one embodiment, the IL-2 mimetics is not capable of binding CD25. In one embodiment, the IL-2 mimetics binds preferentially to an IL-2R comprising the β and γ subunits as compared to an IL-2R comprising the α, β and γ subunits. A non-limitative example of IL-2 mimetics that may be used is NKTR-214 (Nektar Therapeutics).

In one embodiment, the isolated protein, antibody or antigen-binding fragment thereof, fusion protein, nucleic acid or vector according to the present invention is used in combination with a checkpoint inhibitor therapy.

As used herein, a “checkpoint inhibitor therapy” is defined as the administration of at least one checkpoint inhibitor to the subject.

As a cancer treatment, checkpoint inhibitor therapy aims at preventing the activation of inhibitory receptors expressed on T cells by ligands expressed by the tumor cells.

Checkpoint inhibitor therapy thus aims at preventing the inhibition of T cells present in the tumor, i.e., tumor infiltrating T cells, and thus at enhancing the subject immune response towards the tumor cells.

Examples of checkpoint inhibitors are listed hereinabove.

In one embodiment, the at least one checkpoint inhibitor is selected from the group comprising or consisting of inhibitors of PD-1, inhibitors of PD-L1, inhibitors of CTLA-4 and any mixtures thereof.

In one embodiment, the isolated protein, antibody or antigen-binding fragment thereof, fusion protein, nucleic acid or vector according to the present invention is used in combination with a checkpoint agonist therapy.

As used herein, a “checkpoint agonist therapy” is defined as the administration of at least one checkpoint agonist to the subject.

As a cancer treatment, checkpoint agonist therapy aims at activating stimulatory receptors expressed on immune cells present in a tumor. In particular, T-cell agonist therapy aims at enhancing the activation of T cells present in a tumor, i.e., tumor infiltrating T cells, and thus at enhancing the subject immune response towards the tumor cells. Currently, a number of potential targets for checkpoint agonist therapy have been identified.

Examples of checkpoint agonists are listed hereinabove.

In one embodiment, the at least one checkpoint agonist is selected from the group comprising or consisting of agonists of CD137, agonists of OX40, agonists of GITR, agonists of ICOS, agonists of CD27-CD70, agonists of CD40 and any mixtures thereof.

In one embodiment, the isolated protein, antibody or antigen-binding fragment thereof, fusion protein, nucleic acid or vector according to the present invention is used in combination with a second antibody that is specific for an immune receptor or a costimulatory molecule.

Examples of antibodies that are specific for an immune receptor include but are not limited to anti-CTLA4 antibodies (e.g. Ipilimumab), anti-PD-1 antibodies, anti-PD-L1 antibodies, anti-TIM-3 antibodies, anti-LAG-3 antibodies, anti-B7H3 antibodies, anti-B7H4 antibodies, anti-B7H6 antibodies, anti-4-1BB antibodies, anti-TIGIT antibodies, anti-ICOS antibodies, anti-GITR antibodies, anti-CD27-CD70 antibodies, anti-CD40 antibodies, anti-BTLA antibodies, anti-HVEM antibodies, anti-CD160 antibodies, anti-CCR8 antibodies, anti-CEACAM-1 and anti-OX40 antibodies.

In some embodiments, the second antibody is specific for CD137. As used herein the term “CD137” has its general meaning in the art and may also be referred to as Ly63, ILA or 4-1BB. CD137 is a member of the tumor necrosis factor (TNF) receptor family. Members of this receptor family and their structurally related ligands are important regulators of a wide variety of physiologic processes and play an important role in the regulation of immune responses. CD137 is expressed by activated NK cells, T and B lymphocytes and monocytes/macrophages. The gene encodes a 255-amino acid protein with 3 cysteine-rich motifs in the extracellular domain (characteristic of this receptor family), a transmembrane region, and a short N-terminal cytoplasmic portion containing potential phosphorylation sites. Expression in primary cells is strictly activation dependent. The ligand for the receptor is TNFSF9. Human CD137 is reported to bind only to its ligand. Agonists include the native ligand (TNFSF9), aptamers (see McNamara et al. (2008) J. Clin. Invest. 1 18: 376-386), and antibodies.

In another embodiment, the antibody or antigen-binding fragment thereof is bispecific, and is further capable of binding to an immune receptor or to a costimulatory molecule.

Examples of immune receptors include, but are not limited to, CTLA4, PD-1, PD-L1, TIM-3, LAG-3, B7H3, B7H4, B7H6, 4-1BB, TIGIT, ICOS, GITR, CD27-CD70, CD40, BTLA, HVEM, CD160, CCR8, CEACAM-1 and OX40.

Examples of costimulatory molecules include, but are not limited to, B7H3, B7H4, B7H6, 4-1BB and OX40, GITR.

In one embodiment, the isolated protein (in particular the antibody or antigen-binding fragment thereof as described herein), fusion protein, nucleic acid or vector according to the present invention is used in combination with a second antibody that induces, via ADCC, the death of a cell expressing an antigen to which the second antibody binds. In one embodiment, the second antibody (e.g. of IgG1 or IgG3 isotype, in particular of human IgG1 or IgG3 isotype) induces ADCC toward a cell to which the antibody binds. NK cells have an important role in inducing ADCC and increased reactivity of NK cells can be directed to target cells through use of such a second antibody. In one embodiment, the second antibody is specific for a cell surface antigen, e.g., membrane antigen. In some embodiments, the second antibody is specific for a tumor antigen (e.g., molecules specifically expressed by tumor cells), such as CD20, CD52, ErbB2 (or HER2/Neu), CD33, CD22, CD25, MUC-1, CEA, KDR, αVβ3, etc., particularly lymphoma antigens (e.g., CD20).

Accordingly, the present invention also provides methods to enhance the anti-tumor effect of monoclonal antibodies directed against tumor antigen(s).

In one embodiment, ADCC function is specifically augmented, which in turn enhances target cell killing, by sequential administration of an antibody directed against one or more tumor antigens, and an antibody or antigen-binding fragment thereof of the present invention.

In one embodiment, the isolated protein (in particular the antibody or antigen-binding fragment thereof as described herein), fusion protein, nucleic acid or vector according to the present invention is used in combination with a natural ligand of an NK cell activating receptor or an antibody that binds and activates an NK cell activating receptor.

In one embodiment, the at least one further therapeutic agent is an agent that increases the presence of a natural ligand of an NK cell activating receptor on the surface of a target cell (e.g., infected cells, or tumor cells).

As used herein, the term “activating NK receptor” refers to any molecule on the surface of NK cells that, when stimulated, causes a measurable increase in any property or activity known in the art as associated with NK activity, such as cytokine (for example TFN-γ and TNF-α) production, increases in intracellular free calcium levels, the ability to target cells in a redirected killing assay, or the ability to stimulate NK cell proliferation.

Examples of “activating NK receptors” include but are not limited to activating forms of KIR proteins (for example KIR2DS proteins), CD160-TM, NKG2D, IL-2R, IL-12R, IL-15R, IL-18R and IL-21R.

Examples of ligands that act as agonists at activating receptors include, e.g. IL-2, IL-15, IL-21 polypeptides.

In one embodiment, the isolated protein (in particular the antibody or antigen-binding fragment thereof as described herein), fusion protein, nucleic acid or vector according to the present invention is used in combination with a therapeutic vaccine or treatment vaccine.

As used herein, a therapeutic vaccine is defined as the administration of at least one tumor-specific antigen (e.g., synthetic long peptides or SLP), or of the nucleic acid encoding said tumor-specific antigen; the administration of recombinant viral vectors selectively entering and/or replicating in tumor cells; the administration of tumor cells; and/or the administration of immune cells (e.g., dendritic cells) engineered to present tumor-specific antigens and trigger an immune response against these antigens.

As a cancer treatment, therapeutic vaccines aim at enhancing the subject immune response towards the tumor cells.

Examples of therapeutic vaccines aiming at enhancing the subject immune response towards tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; RNA-based vaccines, and dendritic cell vaccines.

In one embodiment, the isolated protein (in particular the antibody or antigen-binding fragment thereof as described herein), fusion protein, nucleic acid or vector according to the present invention is used in combination with an oncolytic virus therapy.

As used herein, an “oncolytic virus therapy” is defined as the administration of at least one oncolytic virus to the subject.

Oncolytic viruses are defined as viruses that preferentially infect and kill cancer cells over normal, non-cancer, cells. As a cancer treatment, oncolytic virus therapy aims at killing cancer cells and/or triggering or enhancing an immune response towards the cancer cells.

Examples of oncolytic viruses include, without being limited to, modified herpes simplex type-1 viruses such as talimogene laherparepvec (or T-VEC) or HSV-1716; modified adenoviruses such as Ad5-DNX-2401; modified measles viruses such as MV-NIS; modified vaccinia viruses (VV) such as vaccinia virus TG6002; and modified polioviruses such as PVS-RIPO.

In one embodiment, the isolated protein (in particular the antibody or antigen-binding fragment thereof as described herein), fusion protein, nucleic acid or vector according to the present invention is used in combination with an adoptive transfer of cells, also referred to as adoptive cell therapy (both also referred to as ACT), such as, for example, an adoptive transfer of T cells or NK cells, also referred to as adoptive T cell therapy or adoptive NK cell therapy, respectively.

As used herein, an “adoptive transfer of cells” or “adoptive cell therapy” is defined as the transfer, for example as an infusion or re-infusion, of immune cells to a subject. As a cancer treatment, the adoptive transfer of immune cells to a subject aims at enhancing the subject immune response towards the cancer cells.

Examples of immune cells that may be used for a cell therapy include without limitation cytotoxic cells (e.g., natural killer (NK) cells, CD8⁺ T cells, and natural killer (NK) cells T cells), effector T cells (e.g., CD4⁺ T cells and CD8⁺ T cells), alpha beta (αβ) T cells, and gamma delta (γδ) T cells, antibody-expressing B cells or other antibody-producing or -presenting cells and dendritic cells.

In one embodiment, the transferred immune cells as described hereinabove are antigen-specific cells. In one embodiment, the transferred immune cells as described hereinabove are antigen-specific immune cells, wherein said antigen is specifically and/or abundantly expressed by cancer cells. In one embodiment, the transferred immune cells as described hereinabove are tumor-specific immune cells, in other words the transferred immune cells as described hereinabove specifically recognize cancer cells or tumor cells through an antigen specifically and/or abundantly expressed by said cancer cells or tumor cells. In one embodiment, the transferred immune cells as described hereinabove are tumor-specific effector T cells. In one embodiment, the transferred immune cells as described hereinabove are tumor-specific CD8⁺ effector T cells, in particular tumor-specific cytotoxic CD8⁺ T cells. In one embodiment, the transferred cells are tumor infiltrating cells (TIL). In one embodiment, the transferred immune cells as described hereinabove are tumor-specific cytotoxic cells. In one embodiment, the transferred immune cells as described hereinabove are tumor-specific NK cells.

Examples of tumor-specific antigens, i.e., antigens that are specifically and/or abundantly expressed by cancer cells include, without being limited to, neoantigens (also referred to as new antigens or mutated antigens), 9D7, ART4, β-catenin, BING-4, Bcr-ab1, BRCA1/2, calcium-activated chloride channel 2, CDK4, CEA (carcinoembryonic antigen), CML66, Cyclin B1, CypB, EBV (Epstein-Barr virus) associated antigens (such as LMP-1, LMP-2, EBNA1 and BARF1), Ep-CAM, EphA3, fibronectin, Gp100/pmel17, Her2/neu, HPV (human papillomavirus) E6, HPV E7, hTERT, IDH1, IDH2, immature laminin receptor, MC1R, Melan-A/MART-1, MART-2, mesothelin, MUC1, MUC2, MUM-1, MUM-2, MUM-3, NY-ESO-1/LAGE-2, p53, PRAME, prostate-specific antigen (PSA), PSMA (prostate-specific membrane antigen), Ras, SAP-1, SART-I, SART-2, SART-3, SSX-2, survivin, TAG-72, telomerase, TGF-βRII, TRP-1/-2, tyrosinase, WT1, antigens of the BAGE family, antigens of the CAGE family, antigens of the GAGE family, antigens of the MAGE family, antigens of the SAGE family, and antigens of the XAGE family.

As used herein, neoantigens (also referred to as new antigens or mutated antigens) correspond to antigens derived from proteins that are affected by somatic mutations or gene rearrangements acquired by the tumors. Neoantigens may be specific to each individual subject and thus provide targets for developing personalized immunotherapies. Examples of neoantigens include for example, without being limited to, the R24C mutant of CDK4, the R24L mutant of CDK4, KRAS mutated at codon 12, mutated p53, the V599E mutant of BRAF and the R132H mutant of IDH1.

In one embodiment, the transferred immune cells as described hereinabove are specific for a tumor antigen selected from the group comprising or consisting of the class of CTAs (cancer/testis antigens, also known as MAGE-type antigens), the class of neoantigens and the class of viral antigens.

As used herein, the class of CTAs corresponds to antigens encoded by genes that are expressed in tumor cells but not in normal tissues except in male germline cells. Examples of CTAs include, without being limited to, MAGE-A1, MAGE-A3, MAGE-A4, MAGE-C2, NY-ESO-1, PRAME and SSX-2.

As used herein, the class of viral antigens corresponds to antigens derived from viral oncogenic proteins. Examples of viral antigens include, without being limited to, HPV (human papillomavirus) associated antigens such as E6 and E7, and EBV (Epstein-Barr virus) associated antigens such as LMP-1, LMP-2, EBNA1 and BARF1.

In one embodiment, the transferred immune cells as described hereinabove are autologous immune cells, in particular autologous T cells. In another embodiment, the transferred immune cells as described hereinabove are allogenic (or allogenous) immune cells, in particular allogenic NK cells.

For example, autologous T cells can be generated ex vivo either by expansion of antigen-specific T cells isolated from the subject or by redirection of T cells of the subject through genetic engineering.

In one embodiment, the immune cells to be infused are modified ex vivo before being infused to the subject.

Methods to isolate T cells from a subject, in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand T cells ex vivo are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71).

In one embodiment, the isolated protein (in particular the antibody or antigen-binding fragment thereof as described herein), fusion protein, nucleic acid or vector according to the present invention is used in combination with a CAR immune cell therapy, in particular a CAR T cell therapy or a CAR NK cell therapy.

As used herein, CAR immune cell therapy is an adoptive cell therapy wherein the transferred cells are immune cells as described hereinabove, such as T cells or NK cells, genetically engineered to express a chimeric antigen receptor (CAR). As a cancer treatment, the adoptive transfer of CAR immune cells to a subject aims at enhancing the subject immune response towards the cancer cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3 zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, the transferred T cells as described hereinabove are CAR T cells. The expression of a CAR allows the T cells to be redirected against a selected antigen, such as an antigen expressed at the surface of cancer cells. In one embodiment, the transferred CAR T cells recognize a tumor-specific antigen.

In another embodiment, the transferred NK cells as described hereinabove are CAR NK cells. The expression of a CAR allows the NK cells to be redirected against a selected antigen, such as an antigen expressed at the surface of cancer cells. In one embodiment, the transferred CAR NK cells recognize a tumor-specific antigen.

Examples of tumor-specific antigens are mentioned hereinabove.

In one embodiment, the transferred CAR T cells or CAR NK cells recognize a tumor-specific antigen selected from the group comprising or consisting of EGFR and in particular EGFRvIII, mesothelin, PSMA, PSA, CD47, CD70, CD133, CD171, CEA, FAP, GD2, HER2, IL-13Rα, αvβ6 integrin, ROR1, MUC1, GPC3, EphA2, CD19, CD21, and CD20.

In one embodiment, the CAR immune cells as described hereinabove are autologous CAR immune cells, in particular autologous CAR T cells. In another embodiment, the CAR immune cells as described hereinabove are allogenic (or allogenous) CAR immune cells, in particular allogenic CAR NK cells.

In one embodiment, the isolated protein (in particular the antibody or antigen-binding fragment thereof as described herein), fusion protein, nucleic acid or vector according to the present invention is used in combination with an antibiotic. Examples of antibiotics include, but are not limited to, penicillins (e.g., penicillin, amoxicillin), tetracyclines (e.g., doxycycline, tetracycline, minocycline), cephalosporins (e.g., cefuroxime, ceftriaxone, cefdinir), quinolones (e.g., ciprofloxacin, levofloxacin, moxifloxacin), lincomycins (e.g., clindamycin, lincomycin), macrolides (e.g., azithromycin, clarithromycin, erythromycin), sulfonamides (e.g., sulfamethoxazole-trimethoprim, sulfasalazine, sulfisoxazole), glycopeptides (e.g., dalbavancin, oritavancin, telavancin, vancomycin), aminoglycosides (e.g., gentamicin, tobramycin, amikacin) and carbapenems (e.g., imipenem, meropenem, doripenem, ertapenem).

In one embodiment, the isolated protein (in particular the antibody or antigen-binding fragment thereof as described herein), fusion protein, nucleic acid or vector according to the present invention is used in combination with an antiviral drug. Examples of antiviral drugs include, but are not limited to, abacavir, acyclovir, adefovir, amantadine, amprenavir, ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever famciclovir, fomivirsen, fosamprenavir, foscarnet, fosfonet, ibacitabine, imunovir, idoxuridine, imiquimod, indinavir, inosine, integrase inhibitor, interferon type III, interferon type II, interferon type I, interferon, lamivudine, lopinavir, loviride, maraviroc, moroxydine, methisazone, nelfinavir, nevirapine, nexavir, nitazoxanide, nucleoside analogues, norvir, oseltamivir, peginterferon alfa-2a, penciclovir, peramivir, pleconaril, podophyllotoxin, protease inhibitor, raltegravir, reverse transcriptase inhibitor, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

In one embodiment, the isolated protein (in particular the antibody or antigen-binding fragment thereof as described herein), fusion protein, nucleic acid or vector according to the present invention is used in combination with an antifungal agent. Examples of antifungal agents include, but are not limited to, polyene antifungals (e.g., amphotericin B, candicidin, filipin, hamycin, natamycin, nystatin, rimocidin), imidazole antifungals (e.g., bifonazole, butoconazole, clotrimazole, econazole, fenticonazole, isoconazole, ketoconazole, luliconazole, miconazole, omoconazole, oxiconazole, sertaconazole, sulconazole, tioconazole), triazole antifungals (e.g., albaconazole, efinaconazole, epoxiconazole, fluconazole, isavuconazole, itraconazole, posaconazole, propiconazole, ravuconazole, terconazole, voriconazole), thiazole antifungals (e.g., abafungin), allylamines, echinocandins (e.g., anidulafungin, caspofungin, micafungin).

In one embodiment, the isolated protein (in particular the antibody or antigen-binding fragment thereof as described herein), fusion protein, nucleic acid or vector according to the present invention is used in combination with an anti-parasitic agent. Examples of anti-parasitic agents include, but are not limited to, broad-spectrum anti-parasitic agents (e.g., nitazoxanide), antiprotozoals (e.g., melarsoprol, eflornithine, metronidazole, tinidazole, miltefosine), anihelminthic (including, without limitation, antinematodes (ancylostoma caninum, mebendazole, pyrantel pamoate, thiabendazole, diethylcarbamazine, ivermectin), anticestodes (e.g., niclosamide, praziquantel, albendazole), antitrematodes (e.g., praziquantel)), antiamoebics (e.g., rifampin, amphotericin B).

Another object of the present invention relates to the use of the isolated protein (in particular the antibody or antigen-binding fragment thereof as described herein) or the fusion protein as described hereinabove with another therapeutic agent as described hereinabove, in the treatment of diseases in a subject in need thereof, wherein said protein, antibody or antigen-binding fragment thereof or fusion protein is used as an adjuvant for the therapeutic agent.

The present invention thus relates to the isolated protein (in particular the antibody or antigen-binding fragment thereof as described herein) or the fusion protein as described hereinabove (preferably in a composition, pharmaceutical composition or medicament), for use as an adjuvant in a cancer therapy. The present invention thus relates to the isolated protein (in particular the antibody or antigen-binding fragment thereof as described herein) or the fusion protein as described hereinabove (preferably in a composition, pharmaceutical composition or medicament), for use as an adjuvant in a therapy for an infectious disease.

In one embodiment, the present invention relates to the use of the isolated protein (in particular the antibody or antigen-binding fragment thereof as described herein) or the fusion protein as described hereinabove, for potentiating an immune response induced by a cancer therapy in a patient in need thereof.

In one embodiment, the isolated protein (in particular the antibody or antigen-binding fragment thereof as described herein) or the fusion protein according to the present invention may be used as immunotherapeutic agent, particularly to treat a wide variety of cancers (e.g., cancers associated with immunosuppression and/or immune exhaustion).

In one embodiment, the isolated protein (in particular the antibody or antigen-binding fragment thereof as described herein) or the fusion protein according to the present invention may potentiate an immune response induced by a cancer therapy in a patient comprising administering said protein (e.g., antibody or fragment thereof) or said fusion protein to a subject in an amount effective to potentiate an immune response induced by the cancer therapy in the patient.

As used herein, the term “adjuvant” refers to a compound or a combination of compounds that potentiates at therapy, such as, for example, a cancer therapy. Adjuvants may increase the effective immune response against low or non-immunogenic tumor cells. In one embodiment, the adjuvant is used with a well-known cancer therapeutic agent in the treatment of cancer and thus potentiates the immune response towards cancer cells. For example, an adjuvant may potentiate an immune response during a cancer therapy, decrease T cell exhaustion (without decreasing T cells activation), increase the survival of T cells, enhance NK cells cytotoxicity, decrease the tumor growth and/or the tumor size, and/or increase in survival, treats or prevents cancer metastasis. In one embodiment, potentiation of a cancer therapy in the presence of an adjuvant, is defined by comparison with a cancer therapy administered alone.

In another embodiment, the isolated protein (in particular the antibody or antigen-binding fragment thereof as described herein) or the fusion protein as described hereinabove can increase or improve the immune response of a subject.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments of any of the aspects, the response is specific for a particular antigen (an “antigen-specific response”), and refers to a response by a CD4 T cell, CD8 T cell, or B cell via their antigen-specific receptor. In some embodiments of any of the aspects, an immune response is a T cell response, such as a CD4⁺ response or a CD8⁺ response. Such responses by these cells can include, for example, cytotoxicity, proliferation, cytokine or chemokine production, trafficking, or phagocytosis, and can be dependent on the nature of the immune cell undergoing the response.

As with other known immunotherapeutic agents, the ability of the isolated protein (in particular the antibody or antigen-binding fragment thereof as described herein) or the fusion protein as described hereinabove, to potentiate an immune response in a patient may have broader therapeutic implications outside the cancer field. For example, it has been proposed that immune potentiating agents may be useful in treating a wide variety of infectious diseases, particularly pathogenic agents which promote immunosuppression and/or immune exhaustion. Also, such immune potentiating agents may be useful in boosting the immunization efficacy of vaccines (e.g., infectious disease and cancer vaccines).

Another object of the present invention relates to the use of the isolated protein (in particular the antibody or antigen-binding fragment thereof as described herein) or of the fusion protein as described hereinabove, or of a nucleic acid or an vector according to the present invention (preferably in a composition, pharmaceutical composition or medicament as described hereinabove) to deplete CD25 expressing Treg cells in a subject in need thereof, wherein a therapeutically effective amount of the isolated protein (in particular the antibody or antigen-binding fragment thereof as described herein), the fusion protein, the nucleic acid, or of the vector of the present invention is to be administered to the subject.

The present invention thus further relates to a method for depleting CD25 expressing Treg cells in a subject in need thereof, comprising administering to the subject an isolated protein (in particular an antibody or antigen-binding fragment thereof as described herein), a fusion protein, a nucleic acid, a vector, a composition, a pharmaceutical composition, or a medicament as described herein.

In one embodiment, the isolated protein (in particular the antibody or antigen-binding fragment thereof as described herein), the fusion protein, the nucleic acid or the vector as described hereinabove (preferably in a composition, pharmaceutical composition or medicament as described hereinabove), is for use to deplete CD25 expressing Treg cells.

In one embodiment, the CD25 expressing Treg cells are tumor infiltrating Tregs.

In one embodiment, the antibody or antigen-binding fragment thereof as described hereinabove for use to deplete CD25 expressing Treg cells is an IgG, preferably an IgG1.

In one embodiment, the antibody or antigen-binding fragment thereof as described hereinabove for use to deplete CD25 expressing Treg cells binds to at least one activating Fcγ Receptor, preferably selected from FcγRI, FcγRIIa, FcγRIII with a high affinity.

In one embodiment, the antibody or antigen-binding fragment thereof as described hereinabove for use to deplete CD25 expressing Treg cells elicits an enhanced ADCC, ADCP and/or CDC response, preferably an increased ADCC and/or ADCP response, more preferably an increased ADCC response.

In one embodiment, the antibody or antigen-binding fragment thereof as described hereinabove for use to deplete CD25 expressing Treg cells does not inhibit the IL-2 signaling via CD25. Thus, in one embodiment, the antibody or antigen-binding fragment thereof as described hereinabove for use to deplete CD25 expressing Treg cells does not inhibit the proliferation and/or activation of CD4⁺ and CD8⁺ T cells (or effector T cells). In another embodiment, the antibody or antigen-binding fragment thereof as described hereinabove for use to deplete CD25 expressing Treg cells does not inhibit the phosphorylation of STAT5a in CD4⁺ and CD8⁺ T cells (or effector T cells).

The present invention further relates to a method of inducing specific lysis of CD25 positive cells without inhibiting IL-2 signaling in T-cells, the method comprising the step of administering to a subject a therapeutically effective amount of an isolated protein (in particular an antibody or antigen-binding fragment thereof as described herein), a fusion protein, a nucleic acid, a vector, a composition, a pharmaceutical composition, or a medicament as disclosed herein.

The present invention further relates to a method of inducing specific lysis of CD25 positive cells by ADCC and/or ADCP without inhibiting IL-2 signaling in T-cells, the method comprising the step of administering to a subject a therapeutically effective amount of an isolated protein (in particular an antibody or antigen-binding fragment thereof as described herein), a fusion protein, a nucleic acid, a vector, a composition, a pharmaceutical composition, or a medicament as disclosed herein.

In some embodiments, the subject is receiving or has received an immunotherapy.

The present invention further relates to a method comprising the step of administering to a subject an immunotherapy, wherein the subject has received or is receiving a therapeutically effective amount of an isolated protein (in particular an antibody or antigen-binding fragment thereof as described herein), a fusion protein, a nucleic acid, a vector, a composition, a pharmaceutical composition, or a medicament as disclosed herein.

In some embodiments, the therapeutically effective amount is an amount effective to induce specific lysis of CD25 positive cells without inhibiting IL-2 signaling in T-cells.

In some embodiments, the therapeutically effective amount is an amount effective to induce specific lysis of CD25 positive cells by ADCC and/or ADCP without inhibiting IL-2 signaling in T-cells.

The present invention further relates to the use of an isolated protein (in particular an antibody or antigen-binding fragment thereof as described herein), a fusion protein, a nucleic acid, a vector as disclosed herein in the manufacture of a medicament for treating diseases, disorders or symptoms as disclosed hereinabove (e.g. cancer or infectious diseases) in a subject in need thereof.

As compared to the anti-CD25 antibodies of the prior art, the antibodies or antigen-binding fragments thereof of the present invention may present at least one of the following advantages:
in some embodiments, the antibody or antigen-binding fragment thereof of the present invention presents an increased affinity for CD25, as compared to the CD25 antibodies of the prior art;
in some embodiments, the antibody or antigen-binding fragment thereof of the present invention presents an increased avidity for CD25, as compared to the CD25 antibodies of the prior art;
in some embodiments, the antibody or antigen-binding fragment thereof of the present invention induces an increased IL-2-dependent activation of T cells in culture, and preferably an increased proliferation of T cells in culture, as compared to the CD25 antibodies of the prior art;
in some embodiments, the antibody or antigen-binding fragment thereof of the present invention induces a lower inhibition of IL-2-induced T cell proliferation in culture as compared to the CD25 antibodies of the prior art;
in some embodiments, the antibody or antigen-binding fragment thereof of the present invention presents an increased ADCC activity on CD25⁺ expressing cells, preferably CD25⁺ expressing T cells and more preferably CD25⁺ expressing Treg cells as compared to the CD25 antibodies of the prior art;
in some embodiments, the antibody or antigen-binding fragment thereof of the present invention presents an increased ADCP activity on CD25⁺ expressing cells, preferably CD25⁺ expressing T cells and more preferably CD25⁺ expressing Treg cells as compared to the CD25 antibodies of the prior art;
in some embodiments, the antibody or antigen-binding fragment thereof of the present invention induces depletion (preferably in vivo depletion) of CD25⁺ expressing cells, preferably CD25⁺ expressing T cells and more preferably CD25⁺ expressing Treg cells, with a higher efficiency as compared to the CD25 antibodies of the prior art;
in some embodiments, the antibody or antigen-binding fragment thereof of the present invention presents a lower immunogenicity as compared to the CD25 antibodies of the prior art;
in some embodiments, the antibody or antigen-binding fragment thereof of the present invention presents a higher stability as compared to the CD25 antibodies of the prior art.

### Examples

The present invention is further illustrated by the following examples.

Materials and Methods

Binding of CD25 Specific mAbs

The CD25 positive SU-DHL1 cell line was incubated with either labeled human IgG1 control, anti-CD25 antibodies (MAB02, MAB09, MAB10, MAB17, MAB18) or Basiliximab at 10 μg/ml for 30 min at 4° C. Cells were washed with PBS and incubated with an APC-labeled goat anti-human IgG for 30 min at 4° C. Cell were then washed again before flow cytometry analysis (CytoFlex Beckman Coulter).

CD25 Positive Cells Specific Lysis by Antibody Dependent Cell Cytotoxicity (ADCC)

Anti-CD25 induced antibody dependent cell cytotoxicity (ADCC) was obtained by coculturing purified NK cells and Calcein-AM-labelled SU-DHL1 (target cells) with human IgG1 control or anti-CD25 antibodies (MAB02, MAB09, MAB10, MAB17, MAB18) or Basiliximab at different concentrations (10⁻⁷, 10⁻⁶, 10-5, 10⁻⁴, 10⁻¹, 10⁻², 10⁻¹, 1 or 10 μg/mL) for 16 h. Cell specific lysis was obtained by colorimetric method, quantifying LDH release in the culture medium. Alternatively, cell death was evaluated by Flow Cytometry with a viability dye staining (7AAD). Percentage of CFSE+7AAD+ cells correspond to the target cells apoptosis.

CD25 Positive Cells Specific Lysis by Antibody Dependent Cell Phagocytosis (ADCP)

Anti-CD25 induced ADCP was obtained by coculturing THP-1 cells (as effector cells) and CFSE-stained SUDHL-1 cells (target cells) and either human IgG1 control or anti-CD25 antibodies (MAB02, MAB09, MAB10, MAB17, MAB18) or Basiliximab at 10 μg/ml for 2 h 30 at 37° C. At this time point, cells were collected, washed and an anti-CD33 APC antibody is added to the coculture. Cells are washed before Flow Cytometry analysis. Percentages of CD33+CFSE+ among CD33+THP1 cells correspond to the level of induced phagocytosis.

IL-2 Binding Assay

SU-DHL1 cells were pre-incubated with human IgG1 control isotype, anti-CD25 antibodies (MAB02, MAB09, MAB10, MAB17, MAB18) or Basiliximab at 1 μg/ml for 1 h at room temperature. Cells were then sequentially incubated for 30 min at room temperature with biotinylated-conjugated IL-2 (125 ng/ml) and APC-coupled streptavidin. A PBS wash was performed between each step of the procedure. Cells were analysed by flow cytometry on a Cytoflex (Beckman Coulter) and data analysed with FlowJo software. Data are expressed as the mean±SEM of the mean fluorescence intensity of APC on gated live SU-DHL1 cells.

Impact on IL-2 Induced T Cell Proliferation

Freshly isolated peripheral mononuclear cells (PBMC) were cultured in RPMI medium (10% FCS, 2% glutamin, 1% antibiotics) completed with PHA at 5 μg/ml for 72 h. PBMC were stained with CFSE, starved 24 h and activated T cells were then isolated by magnetic cell sorting. T cells were cultured 72 h with IL-2 at 50 UI/ml and either anti-CD25 antibodies (MAB02, MAB09, MAB10, MAB17, MAB18), Basiliximab, 7G7B6 or MA-251 at 1 μg/ml. Cell division was followed by flow cytometry.

Treg Cells Depletion

PBMC (2×10⁶/ml) were incubated with human IgG1 control isotype, anti-CD25 antibodies of the present invention (MAB02, MAB09, MAB10, MAB17, MAB18) or Basiliximab (10 μg/ml) plus anti-CD3/anti-CD28-coupled beads (Dako). After 6 days of incubation, labelings were performed using a mix of anti-CD8-FITC, -CD4-PE, CD39-PerCP-Cy5.5, -CD127-PE-Cy7, -CD3-APC and CD45-Pacific Blue antibodies. Cells were analyzed by flow cytometry on a Cytoflex (Beckman Coulter) and data analyzed with FlowJo software. Tregs were identified as the CD3⁺CD4⁺CD39⁺CD127^{low/-} cell population. CD4⁺ T effector cells were identified as the CD3⁺CD4⁺(−) CD3⁺CD4⁺CD39⁺CD127^{low/-} cell population. CD8⁺ T effector cells were identified as the CD3⁺CD8⁺ cell population. Results are expressed as the mean %±SEM of Tregs, CD4⁺ T effector cells or CD8+ T effector cells within the CD45+ lymphocyte population.

### RESULTS

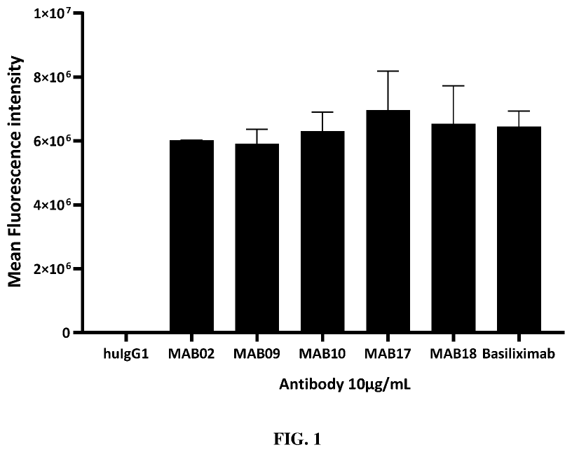

As shown in FIG. 1, the anti-CD25 antibodies of the present invention (MAB02, MAB09, MAB10, MAB17, MAB18) are capable of binding to CD25 on SU-DHL1 cells in a CD25 binding assay. The measured binding was equivalent to the one measured for Basiliximab.

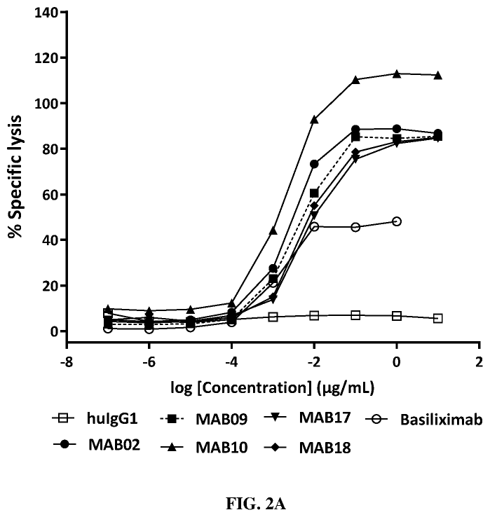

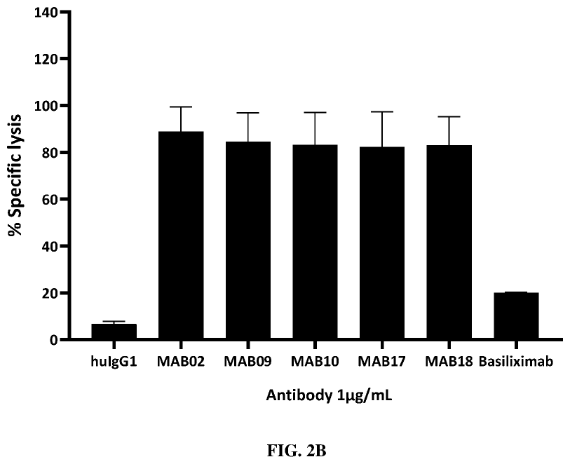

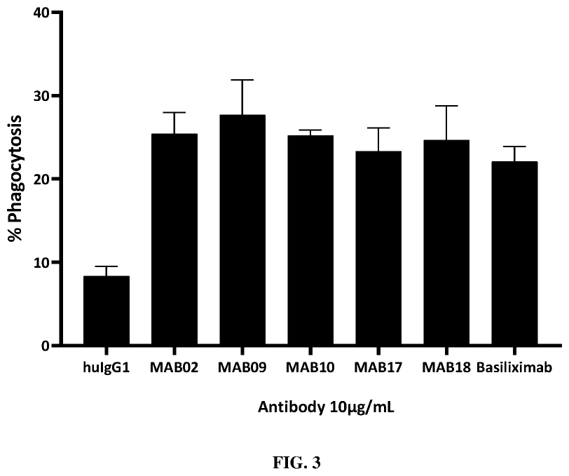

In addition, FIG. 2 demonstrates that the anti-CD25 antibodies of the present invention induce efficient specific lysis of CD25 positive cells by ADCC (FIGS. 2A, 2B). Interestingly, these results show that the anti-CD25 antibodies of the present invention are more efficient to induce cell lysis than Basiliximab at concentrations above 10⁻¹ and 1 μg/mL (FIGS. 2A-2B). Moreover, the anti-CD25 antibodies of the present invention also induce cell lysis by ADCP, as shown in FIG. 3.

The ability of the anti-CD25 antibodies of the present invention to impact IL-2 signaling was next evaluated.

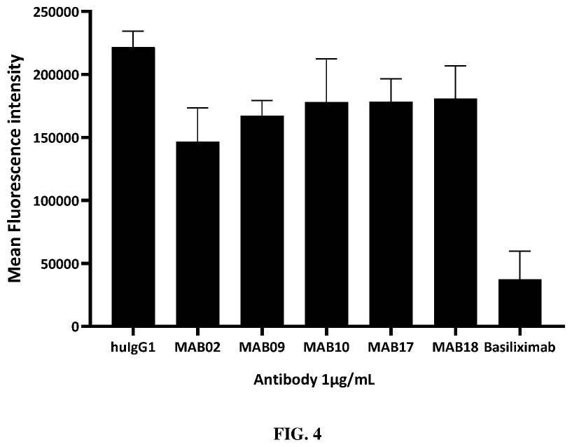

First, the ability of the anti-CD25 antibodies of the present invention to block IL-2 binding was assessed with an IL-2 binding assay. As shown in FIG. 4, contrary to Basiliximab, the anti-CD25 antibodies of the present invention have a limited impact on IL-2 binding on SU-DHL1 cells, when used at 1 μg/mL.

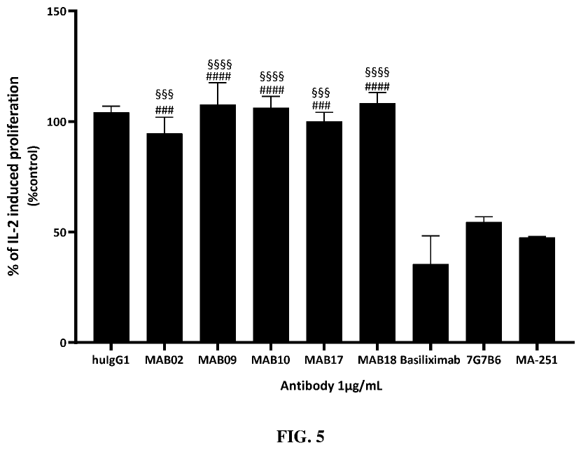

The ability of the anti-CD25 antibodies of the present invention to impact the IL-2 induced effector T cell proliferation was measured. FIG. 5 demonstrates that the anti-CD25 antibodies of the present invention have no significant impact on IL-2-induced effector T cell proliferation, whereas Basiliximab, 7G7B6 and MA-251 all decrease the effector T cell proliferation to at least around 50%.

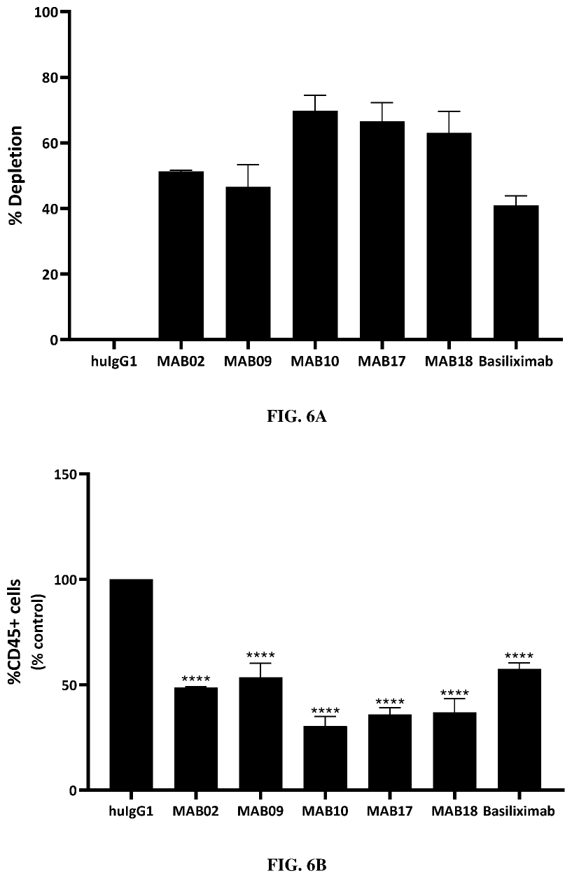

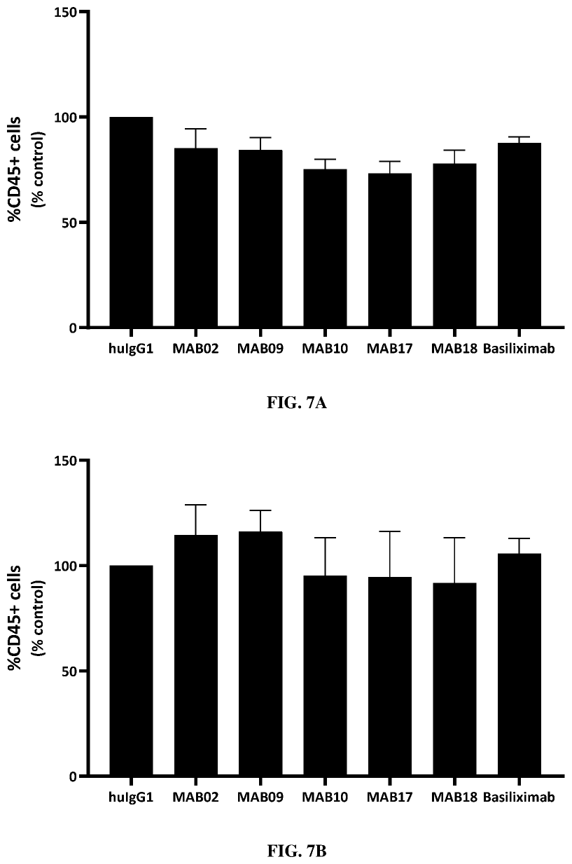

Then, the ability of the anti-CD25 antibodies of the present invention to induce in vitro depletion of Treg cells from anti-CD3+anti-CD28 activated human peripheral mononuclear cells was measured and compared to that of a control antibody (huIgG1) or Basiliximab. As shown in FIGS. 6A and 6B, the anti-CD25 antibodies of the present invention deplete Treg cells by at least 40%. Some anti-CD25 antibodies of the present invention are even more efficient than Basiliximab for depleting Treg cells. Importantly, this effect is specific to Treg cells, as no cell depletion was observed for CD4+ effector T cells nor for CD8+ effector T cells (FIGS. 7A, 7B).

Taken together, these results demonstrate that the anti-CD25 antibodies of the present invention are non-blocking antibodies that have significantly less impact on IL-2 activity than 7G7B6 and MA-251, and are capable of efficiently and specifically deplete Treg cells.

Therefore, these results demonstrate the therapeutic potential of the antibodies of the present invention for treating cancer.

1-15. (canceled)

16. An isolated anti-human CD25 antibody or antigen-binding fragment thereof,
wherein the variable region of the heavy chain (VH) comprises the three following complementary-determining regions (CDRs):
CDR1:
(SEQ ID NO: 1)
X₁HAMA, wherein X₁ is N or S;
CDR2:
(SEQ ID NO: 2)
YISYDGDNTYYX₂DSVKG wherein X₂ is R or A;
and
CDR3:
(SEQ ID NO: 3)
GGNSGYD;
or any CDR having an amino acid sequence that shares at least about 70% of identity with SEQ ID NO: 1-3; and
wherein the variable region of the light chain (VL) comprises the three following CDRs:
CDR1:
(SEQ ID NO: 4)
X₃X₄SQNVNKFLN wherein X₃ is K or R and wherein
X₄ is G or A;
CDR2:
(SEQ ID NO: 5)
GTNSLQX₅ wherein X₅ is T or S;
and
CDR3:
(SEQ ID NO: 6)
QQYTSWPWT,
or any CDR having an amino acid sequence that shares at least about 70% of identity with SEQ ID NO: 4-6; and
wherein when X₁ is N and X₂ is R, X₃ is R and/or X₅ is S; and
wherein when X₃ is K and X₅ is T, X₁ is S and/or X₂ is A.

17. The isolated anti-human CD25 antibody or antigen-binding fragment thereof according to claim 16,
wherein the VH of said antibody or antigen-binding fragment thereof comprises the following CDRs:
CDR1:
(SEQ ID NO: 7)
NHAMA;
CDR2:
(SEQ ID NO: 8)
YISYDGDNTYYRDSVKG;
and
CDR3:
(SEQ ID NO: 3)
GGNSGYD;
and wherein the VL of said antibody or antigen-binding fragment thereof comprises the following CDRs:
CDR1:
(SEQ ID NO: 9)
RASQNVNKFLN;
CDR2:
(SEQ ID NO: 10)
GTNSLQS;
and
CDR3:
(SEQ ID NO: 6)
QQYTSWPWT.

18. The isolated anti-human CD25 antibody or antigen-binding fragment thereof according to claim 16,
wherein the VH of said antibody or antigen-binding fragment thereof comprises the following CDRs:
CDR1:
(SEQ ID NO: 7)
NHAMA;
CDR2:
(SEQ ID NO: 11)
YISYDGDNTYYADSVKG;
and
CDR3:
(SEQ ID NO: 3)
GGNSGYD;
and wherein the VL of said antibody or antigen-binding fragment thereof comprises the following CDRs:
CDR1:
(SEQ ID NO: 9)
RASQNVNKFLN;
CDR2:
(SEQ ID NO: 10)
GTNSLQS;
and
CDR3:
(SEQ ID NO: 6)
QQYTSWPWT.

19. The isolated anti-human CD25 antibody or antigen-binding fragment thereof according to claim 16,
wherein the VH of said antibody or antigen-binding fragment thereof comprises the following CDRs:
CDR1:
(SEQ ID NO: 7)
NHAMA
CDR2:
(SEQ ID NO: 11)
YISYDGDNTYYADSVKG;
and
CDR3:
(SEQ ID NO: 3)
GGNSGYD;
and wherein the VL of said antibody or antigen-binding fragment thereof comprises the following CDRs:
CDR1:
(SEQ ID NO: 12)
KGSQNVNKFLN;
CDR2:
(SEQ ID NO: 13)
GTNSLQT;
and
CDR3:
(SEQ ID NO: 6)
QQYTSWPWT.

20. The isolated anti-human CD25 antibody or antigen-binding fragment thereof according to claim 16,
wherein the VH of said antibody or antigen-binding fragment thereof comprises the following CDRs:
CDR1:
(SEQ ID NO: 14)
SHAMA;
CDR2:
(SEQ ID NO: 11)
YISYDGDNTYYADSVKG;
and
CDR3:
(SEQ ID NO: 3)
GGNSGYD
and wherein the VL of said antibody or antigen-binding fragment thereof comprises the following CDRs:
CDR1
(SEQ ID NO: 12)
KGSQNVNKFLN;
CDR2:
(SEQ ID NO: 13)
GTNSLQT;
and
CDR3:
(SEQ ID NO: 6)
QQYTSWPWT.

21. The isolated anti-human CD25 antibody or antigen-binding fragment thereof according to claim 16,
wherein the VH of said antibody or antigen-binding fragment thereof comprises the following CDRs:
CDR1:
(SEQ ID NO: 14)
SHAMA;
CDR2:
(SEQ ID NO: 11)
YISYDGDNTYYADSVKG;
and
CDR3:
(SEQ ID NO: 3)
GGNSGYD,
and wherein the VL of said antibody or antigen-binding fragment thereof comprises the following CDRs:
CDR1:
(SEQ ID NO: 9)
RASQNVNKFLN;
CDR2:
(SEQ ID NO: 10)
GTNSLQS;
and
CDR3:
(SEQ ID NO: 6)
QQYTSWPWT.

22. The isolated anti-human CD25 antibody or antigen-binding fragment thereof according to claim 16, which is chimeric, humanized or human.

23. The isolated anti-human CD25 antibody or antigen-binding fragment thereof according to claim 16, wherein said antibody or antigen-binding fragment is monoclonal.

24. The isolated anti-human CD25 antibody or antigen-binding fragment thereof according to claim 16, wherein said antibody or antigen-binding fragment mediates antibody dependent cellular cytotoxicity, complement dependent cytotoxicity and/or antibody-dependent phagocytosis.

25. The isolated anti-human CD25 antibody or antigen-binding fragment thereof according to claim 16, being bispecific.

26. The isolated anti-human CD25 antibody or antigen-binding fragment thereof according to claim 16, being conjugated with a drug.

27. A fusion protein comprising the isolated anti-human CD25 antibody or the antigen-binding fragment thereof according to claim 16.

28. A nucleic acid encoding the isolated anti-human CD25 antibody or antigen-binding fragment thereof according to claim 16 or a fusion protein comprising said isolated anti-human CD25 antibody or an antigen-binding fragment thereof.

29. A pharmaceutical composition comprising the isolated anti-human CD25 antibody or antigen-binding fragment thereof according to claim 16 or a fusion protein comprising said isolated anti-human CD25 antibody or an antigen-binding fragment thereof, and at least one pharmaceutically acceptable excipient.

30. A method for treating a cancer or an infectious disease in a subject in need thereof, comprising administering to the subject
the isolated anti-human CD25 antibody or an antigen-binding fragment thereof according to claim 16, or
a fusion protein comprising said isolated anti-human CD25 antibody or an antigen-binding fragment thereof, or
a pharmaceutical composition comprising said isolated anti-human CD25 antibody or an antigen-binding fragment thereof and at least one pharmaceutically acceptable excipient.

31. The method according to claim 30, wherein said isolated anti-human CD25 antibody or antigen-binding fragment thereof, said fusion protein or said pharmaceutical composition is administered in combination with an immunotherapy.

32. A method of inducing specific lysis of CD25 positive cells without inhibiting IL-2 signaling in T-cells, the method comprising the step of administering to a subject:
a therapeutically effective amount of the isolated anti-human CD25 antibody or antigen-binding fragment thereof according to claim 16, or
a therapeutically effective amount of a fusion protein comprising said isolated anti-human CD25 antibody or antigen-binding fragment thereof, or
a therapeutically effective amount of a pharmaceutical composition comprising said isolated anti-human CD25 antibody or antigen-binding fragment thereof and at least one pharmaceutically acceptable excipient.

33. The method according to claim 32, wherein the subject is receiving or has received an immunotherapy.

34. A method comprising the step of administering to a subject an immunotherapy, wherein the subject has received or is receiving
a therapeutically effective amount of the isolated anti-human CD25 antibody or antigen-binding fragment thereof according to claim 16, or
a therapeutically effective amount of a fusion protein comprising said isolated anti-human CD25 antibody or antigen-binding fragment thereof, or
a therapeutically effective amount of a pharmaceutical composition comprising said isolated anti-human CD25 antibody or antigen-binding fragment thereof and at least one pharmaceutically acceptable excipient.

35. The method according to claim 34, wherein the therapeutically effective amount is an amount effective to induce specific lysis of CD25 positive cells without inhibiting IL-2 signaling in T-cells.


SEQUENCE LISTING (43 sequences)

SEQ ID NO: 1 - protein, 5 aa, Artificial Sequence
  SITE 1: X is N or S
  CDR1 VH
XHAMA

SEQ ID NO: 2 - protein, 17 aa, Artificial Sequence
  CDR2 VH
  SITE 12: X is R or A
YISYDGDNTYYXDSVKG

SEQ ID NO: 3 - protein, 7 aa, Artificial Sequence
  CDR3 VH
GGNSGYD

SEQ ID NO: 4 - protein, 11 aa, Artificial Sequence
  SITE 1: X is K or R
  CDR1 VL
  SITE 2: X is G or A
XXSQNVNKFLN

SEQ ID NO: 5 - protein, 7 aa, Artificial Sequence
  CDR2 VL
  SITE 7: X is T or S
GTNSLQX

SEQ ID NO: 6 - protein, 9 aa, Artificial Sequence
  CDR3 VL
QQYTSWPWT

SEQ ID NO: 7 - protein, 5 aa, Artificial Sequence
  CDR1 VH
NHAMA

SEQ ID NO: 8 - protein, 17 aa, Artificial Sequence
  CDR2 VH
YISYDGDNTYYRDSVKG

SEQ ID NO: 9 - protein, 11 aa, Artificial Sequence
  CDR1 VL
RASQNVNKFLN

SEQ ID NO: 10 - protein, 7 aa, Artificial Sequence
  CDR2 VL
GTNSLQS

SEQ ID NO: 11 - protein, 17 aa, Artificial Sequence
  CDR2 VH
YISYDGDNTYYADSVKG

SEQ ID NO: 12 - protein, 11 aa, Artificial Sequence
  CDR1 VL
KGSQNVNKFLN

SEQ ID NO: 13 - protein, 7 aa, Artificial Sequence
  CDR2 VL
GTNSLQT

SEQ ID NO: 14 - protein, 5 aa, Artificial Sequence
  CDR1 VH
SHAMA

SEQ ID NO: 15 - protein, 11 aa, Artificial Sequence
  CDR1 VL
RGSQNVNKFLN

SEQ ID NO: 16 - protein, 11 aa, Artificial Sequence
  CDR1 VL
KASQNVNKFLN

SEQ ID NO: 17 - protein, 116 aa, Artificial Sequence
  VH
  SITE 31: X is N or S
  SITE 42: X is K or G
  SITE 61: X is R or A
  SITE 75: X is A or S
  SITE 76: X is Q or K
  SITE 77: X is S or N
QVQLVESGGGVVQPGRSLRLSCAASGFTFSXHAMAWVRQAPXKGLEWVAYISYDGDNTYYXDSVKGRFTISRDNXXXTLYLQMNSLRAEDTAVYYCTTGGNSGYDWGQGTLVTVSS

SEQ ID NO: 18 - protein, 107 aa, Artificial Sequence
  VL
  SITE 24: X is K or R
  SITE 25: X is G or A
  SITE 40: X is L or P
  SITE 42: X is E or K
  SITE 56: X is T or S
  SITE 71: X is Y or F
DIQMTQSPSSLSASVGDRVTITCXXSQNVNKFLNWYQQKXGXAPRRLIYGTNSLQXGVPSRFSGSGSGTDXTLTISSLQPEDFATYYCQQYTSWPWTFGQGTKLEIK

SEQ ID NO: 19 - protein, 116 aa, Artificial Sequence
  VH
QVQLVESGGGVVQPGRSLRLSCAASGFTFSNHAMAWVRQAPKKGLEWVAYISYDGDNTYYRDSVKGRFTISRDNAQSTLYLQMNSLRAEDTAVYYCTTGGNSGYDWGQGTLVTVSS

SEQ ID NO: 20 - protein, 116 aa, Artificial Sequence
  VH
QVQLVESGGGVVQPGRSLRLSCAASGFTFSNHAMAWVRQAPGKGLEWVAYISYDGDNTYYRDSVKGRFTISRDNAKSTLYLQMNSLRAEDTAVYYCTTGGNSGYDWGQGTLVTVSS

SEQ ID NO: 21 - protein, 116 aa, Artificial Sequence
  VH
QVQLVESGGGVVQPGRSLRLSCAASGFTFSNHAMAWVRQAPGKGLEWVAYISYDGDNTYYADSVKGRFTISRDNAKSTLYLQMNSLRAEDTAVYYCTTGGNSGYDWGQGTLVTVSS

SEQ ID NO: 22 - protein, 116 aa, Artificial Sequence
  VH
QVQLVESGGGVVQPGRSLRLSCAASGFTFSSHAMAWVRQAPGKGLEWVAYISYDGDNTYYADSVKGRFTISRDNSKSTLYLQMNSLRAEDTAVYYCTTGGNSGYDWGQGTLVTVSS

SEQ ID NO: 23 - protein, 116 aa, Artificial Sequence
  VH
QVQLVESGGGVVQPGRSLRLSCAASGFTFSSHAMAWVRQAPGKGLEWVAYISYDGDNTYYADSVKGRFTISRDNSKNTLYLQMNSLRAEDTAVYYCTTGGNSGYDWGQGTLVTVSS

SEQ ID NO: 24 - protein, 107 aa, Artificial Sequence
  VL
DIQMTQSPSSLSASVGDRVTITCKGSQNVNKFLNWYQQKLGEAPRRLIYGTNSLQTGVPSRFSGSGSGTDYTLTISSLQPEDFATYYCQQYTSWPWTFGQGTKLEIK

SEQ ID NO: 25 - protein, 107 aa, Artificial Sequence
  VL
DIQMTQSPSSLSASVGDRVTITCRASQNVNKFLNWYQQKPGKAPRRLIYGTNSLQSGVPSRFSGSGSGTDYTLTISSLQPEDFATYYCQQYTSWPWTFGQGTKLEIK

SEQ ID NO: 26 - protein, 107 aa, Artificial Sequence
  VL
DIQMTQSPSSLSASVGDRVTITCRASQNVNKFLNWYQQKPGKAPRRLIYGTNSLQSGVPSRFSGSGSGTDFTLTISSLQPEDFATYYCQQYTSWPWTFGQGTKLEIK

SEQ ID NO: 27 - protein, 19 aa, Artificial Sequence
  Leader sequence
MDIRLSLAFLVLFIKGVQC

SEQ ID NO: 28 - protein, 19 aa, Artificial Sequence
  Leader sequence
MAAVQLLGLLLLWLPAMRC

SEQ ID NO: 29 - DNA, 348 bp, Artificial Sequence
  VH
caagtgcagctggtcgaatccggaggcggagtggtccagcccggcagatctctgagactgagctgtgctgcctccggcttcacattcagcaaccacgctatggcttgggtgagacaagcccctaagaagggactggaatgggtggcctacatcagctacgacggcgacaacacatactatagggacagcgtcaagggaaggttcactatctctagggacaatgcccagagcactctgtatctgcagatgaattctctgagggccgaggatacagccgtctactactgcacaactggcggcaatagcggctacgattggggccaaggcactctggtgacagtgagcagc

SEQ ID NO: 30 - DNA, 348 bp, Artificial Sequence
  VH
caagtgcagctggtggaaagcggaggaggcgtcgtgcagcccggaaggtctctgagactgagctgtgccgccagcggcttcacattcagcaaccacgccatggcttgggtgagacaagcccccggcaaaggactggagtgggtggcctacatctcctacgacggcgacaacacttactatagggacagcgtcaagggcagattcacaatctctagggacaacgccaagagcactctgtatctgcagatgaactctctgagggctgaggatacagccgtgtactactgcactactggcggcaacagcggctatgattggggccaaggcactctggtcactgtgagcagc

SEQ ID NO: 31 - DNA, 348 bp, Artificial Sequence
  VH
caagtgcagctggtggaatccggaggcggcgtggtgcaacccggcagatctctgagactgagctgtgccgccagcggcttcactttcagcaatcacgccatggcttgggtgagacaagcccccggcaagggactggagtgggtcgcctacatctcctacgacggcgacaacacatactacgccgacagcgtcaagggaaggttcactatctctagggacaatgccaagagcacactgtatctgcagatgaactctctgagggccgaggacactgccgtgtactactgcacaactggcggcaatagcggctacgattggggccaaggcactctggtgacagtgagcagc

SEQ ID NO: 32 - DNA, 348 bp, Artificial Sequence
  VH
caagtgcagctggtggagagcggcggaggagtggtgcaacccggaaggtctctgagactgagctgtgccgccagcggctttactttcagcagccacgctatggcttgggtgaggcaagcccccggcaagggactggagtgggtcgcctacatctcctacgacggcgacaacacatactacgccgacagcgtcaagggaaggttcacaatctctagggacaacagcaagagcactctgtatctgcagatgaactctctgagagccgaggacacagccgtctactactgcacaactggcggcaatagcggctacgattggggccaaggcactctggtgacagtgagcagc

SEQ ID NO: 33 - DNA, 348 bp, Artificial Sequence
  VH
caagtgcagctggtggaaagcggaggcggcgtggtgcaacccggcagatctctgagactgagctgtgccgccagcggcttcacattcagctcccacgccatggcttgggtgagacaagcccccggcaaaggactggagtgggtcgcctacatctcctacgacggcgacaacacatactacgccgatagcgtgaagggaaggttcactatctctagggacaacagcaagaacactctgtatctgcagatgaactctctgagggctgaggatacagccgtctactactgcacaactggcggcaatagcggctatgattggggccaaggcactctggtgacagtgagcagc

SEQ ID NO: 34 - DNA, 321 bp, Artificial Sequence
  VL
gatattcagatgacacagagccctagctctctgagcgctagcgtgggagatagggtgacaatcacatgcaagggcagccagaacgtgaacaagtttctgaactggtatcagcagaagctgggcgaggcccctaggaggctgatctacggcacaaactctctgcagactggcgtcccaagcagatttagcggcagcggaagcggcactgactacacactgacaatcagctctctgcagccagaggacttcgccacatactactgccagcagtacacaagctggccttggactttcggccaaggcactaagctggagatcaag

SEQ ID NO: 35 - DNA, 321 bp, Artificial Sequence
  VL
gatatccagatgactcagagcccttcctctctgagcgctagcgtgggagatagggtgactatcacatgtagggccagccagaacgtcaacaagtttctgaactggtatcagcagaagcccggcaaggccccaaggaggctgatctacggcactaactctctgcagagcggcgtgccaagcagatttagcggcagcggaagcggcactgactacacactgactatcagctctctgcagccagaggacttcgccacatactactgccagcagtacactagctggccttggacattcggccaaggcacaaagctggagatcaaa

SEQ ID NO: 36 - DNA, 321 bp, Artificial Sequence
  VL
gacatccagatgacacagagccctagctctctgagcgctagcgtgggagatagggtgacaatcacatgtagggccagccagaacgtgaacaagtttctgaactggtatcagcagaagcccggcaaggcccctaggaggctgatctacggcacaaattctctgcagagcggcgtgcctagcagatttagcggcagcggaagcggcactgacttcacactgactatcagctctctgcagccagaggacttcgccacatactactgccagcagtacacaagctggccttggacattcggccaaggcacaaagctggagatcaag

SEQ ID NO: 37 - DNA, 57 bp, Artificial Sequence
  Leader sequence
atggacatcaggctcagcttggctttccttgtccttttcataaaaggtgtccagtgt

SEQ ID NO: 38 - DNA, 57 bp, Artificial Sequence
  Leader sequence
atggctgcagttcaactcttagggctgctgctgctttggctcccagccatgagatgt

SEQ ID NO: 39 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 40 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 41 - protein, 116 aa, Artificial Sequence
  VH
  SITE 42: X is K or G
  SITE 76: X is Q or K
QVQLVESGGGVVQPGRSLRLSCAASGFTFSNHAMAWVRQAPXKGLEWVAYISYDGDNTYYRDSVKGRFTISRDNAXSTLYLQMNSLRAEDTAVYYCTTGGNSGYDWGQGTLVTVSS

SEQ ID NO: 42 - protein, 107 aa, Artificial Sequence
  VL
  SITE 71: X is Y or F
DIQMTQSPSSLSASVGDRVTITCRASQNVNKFLNWYQQKPGKAPRRLIYGTNSLQSGVPSRFSGSGSGTDXTLTISSLQPEDFATYYCQQYTSWPWTFGQGTKLEIK

SEQ ID NO: 43 - protein, 116 aa, Artificial Sequence
  VH
  SITE 77: X is S or N
QVQLVESGGGVVQPGRSLRLSCAASGFTFSSHAMAWVRQAPGKGLEWVAYISYDGDNTYYADSVKGRFTISRDNSKXTLYLQMNSLRAEDTAVYYCTTGGNSGYDWGQGTLVTVSS